(19) United States
(12) Patent Application Publication
(10) Pub. No.: US 2017/0044208 A1
(43) Pub. Date: Feb. 16, 2017

(54) SELECTIVE GLUCOCORTICOID RECEPTOR LIGANDS

(71) Applicant: The University of Manchester, Manchester (GB)

(72) Inventors: William David Ray, Manchester (GB); William Dawson, Alton (GB); Jane Helen Payne, Hale Cheshire (GB)

(21) Appl. No.: 15/036,407

(22) PCT Filed: Nov. 12, 2014
(86) PCT No.: PCT/GB2014/053351

§ 371 Date: May 12, 2016

(30) Foreign Application Priority Data
Nov. 14, 2013 (GB) 1320091.0

(51) Int. Cl. C07J 41/00; C07J 43/00

(52) U.S. Cl. CPC C07J 41/0066; C07J 43/003

(57) ABSTRACT

Described herein are certain steroid derivative compounds, for example of formula (I): wherein X¹, X², X³ L, and Ar are as defined herein, pharmaceutical compositions comprising such compounds, the use of such compounds and compositions to specifically target glucocorticoid action, and the use of such compounds and compositions in the treatment of acute and chronic inflammatory conditions, in particular rheumatoid arthritis, haematological and other malignancies, and for causing immunosuppression in the prevention or treatment of transplant rejection, as well as methods of preparing such compounds.

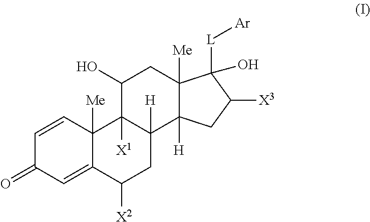

### TECHNICAL FIELD

The present invention pertains generally to the field of therapeutic compounds, and more specifically to certain steroid derivatives. The present invention also pertains to pharmaceutical compositions comprising such compounds, to the use of such compounds and compositions to specifically target glucocorticoid action, and to the use of such compounds and compositions in the treatment of acute and chronic inflammatory conditions, in particular rheumatoid arthritis, haematological and other malignancies, and for causing immunosuppression in the prevention or treatment of transplant rejection, as well as methods of preparing such compounds.

### BACKGROUND

Glucocorticoids (GCs) are highly potent anti-inflammatory agents, but also exert important effects on carbohydrate metabolism, which results in off-target phenomena including diabetes, and obesity, and are catabolic for bone, leading to osteoporosis. Therefore there is considerable interest in identifying how the broad spectrum of glucocorticoid effects can be restricted, to retain the beneficial anti-inflammatory actions, but minimise metabolic, off-target effects. Glucocorticoids act through the Glucocorticoid Receptor (GR), a member of the nuclear receptor superfamily. The GR is a ligand-activated transcription factor, rapidly translocating to the nucleus on activation to bind to target genes.

A number of mechanisms of action have been proposed to explain GR regulation of target gene expression. These include homodimeric direct binding to DNA elements, and binding to other transcription factors in a tethering arrangement. Homodimeric binding to so-called glucocorticoid response elements (GRE) is well-characterised to explain the metabolic actions of GR, whereas binding to, and opposing the action of the NFkB transcription factor explains the majority of the anti-inflammatory actions.

Glucocorticoid Receptor Structure

Glucocorticoids (GC) exert their effects through the glucocorticoid receptor (GR, or NR3C1), a member of the nuclear hormone receptor superfamily. The GR comprises three major functional domains, an N-terminal transactivation domain (NTD), a central DNA-binding domain (DBD), and C-terminal ligand-binding domain (LBD). The DBD and the LBD are linked by a hinge region.

The LBD adopts a complex globular tertiary structure, including eleven a helices and four short p sheets that fold as a central pocket for ligands. The LBD gates ligand access, and also recruits chaperones and coactivators. There is a transcriptional activation function-2(AF2) residue towards its C-terminal end. The AF2 consists of residues 526-556 and has significant ligand-dependent function, acting to recruit co-activator complexes with the motif LXXLL.

Glucocorticoid Receptor Function

In the absence of ligand, GR primarily resides in the cytoplasm as part of a multisubunit complex, including Hsp90, Hsp70, Hsp40, immunophilins, CyP40, and P23. In response to GCs, the GR complex rapidly undergoes a conformational change and subsequently dissociates from the heat shock proteins. Subsequently, the ligand bound GR translocates into the nucleus, driven by the dynein motor protein.

Activated GR binds to consensus elements in the host cell genome to activate or repress gene transcription. These sites are cell-type specific, and are determined, in part, by chromatin structure. Multiple mechanisms have been inferred to explain anti-inflammatory GR action, but the major mechanism is inhibition of NFkB transcription factor function (or transrepression), with the GR operating as a tethered protein bound to the DNA-bound NFkB. In contrast, the metabolic actions of GR, mainly affecting glucose metabolism, and bone homeostasis, are mediated by a homodimeric GR binding to a consensus glucocorticoid response element (or transactivation). Therefore, as different mechanisms appear to govern the desirable anti-inflammatory actions of GR compared to the off-target metabolic actions this raises the possibility of developing ligands capable of dissociating between the mechanisms, and so yielding novel, better tolerated drugs. A similar approach targeting the oestrogen receptor (ER) has resulted in application of selective ER agonists in the clinic (Raloxifene, Tamoxifen).

Glucocorticoid Transactivation and Transrepression: Two Mechanisms of Action

The transrepression and transactivation functions of the GR can be separated. The crystal structure of the GR ligand-binding domain (LBD) on binding either an agonist or an antagonist has now been solved. The alpha helices generate a hydrophobic pocket consisting of a longitudinal cleft, as well as a side-pocket. On binding an agonist, it appears that the C-terminal helix moves to enclose the ligand, generating a new composite surface. However, when binding an antagonist, helix 12 is clearly in a different position and hence the activation surface differs from that of an agonist. Computer modelling and mutagenesis studies of the GR-LBD have suggested a number of important amino acid which bonds with the steroid D-ring and its associated functional groups. Subtle changes to the amino acids Met560 and Tyr735 cause the receptor to retain high affinity binding of ligands, but show a selective loss of transactivation. Furthermore, changes to the functional groups attached to C-17 in the steroid D-ring, predicted to interact with the ligand binding pocket and Met560and Tyr735, results in a selective loss of transactivation in vitro.

Glucocorticoid Induced Osteoporosis

In clinical practice glucocorticoid induced osteoporosis (GIOP) is widely feared, both by patients, and their medical carers. Up to 50% of patients on long-term glucocorticoid treatment will develop GIOP. The pathogenesis of GIOP is complex, including both skeletal, and extraskeletal effects e.g. suppression of sex hormones, and impaired calcium absorption. In vivo GC inhibit osteoblast function, by impairing their generation, and promoting apoptosis. In contrast, the effects of GC on osteoclasts are contentious. In some studies Gc promote activity and longevity of osteoclasts, whereas in others they promote osteoclast apoptosis. There may be important differences seen between different animal models, and humans. Without wishing to be bound by theory, it therefore appears that the major mechanism explaining glucocorticoid-induced osteoporosis in humans is inhibition of osteoblast function.

Glucocorticoid Effects on Glucose Metabolism

Glucocorticoids act on the liver to promote synthesis of glucose from amino acids (gluconeogenesis), and on both the liver and peripheral tissues to oppose the actions of insulin. As a consequence, hyperglycaemia results. The opposition of insulin action also results in disordered lipid metabolism, and the final result of these derangements to energy homeostasis is the acceleration of atherosclerosis. Indeed, in the early trials of synthetic GC administration for rheumatoid arthritis an early indicator of adverse effects was accelerated cardiovascular disease, with premature death. Therefore, tracking the plasma glucose response to GC administration provides a useful predictor of the global impact on energy metabolism, and a surrogate for accelerated atherosclerosis.

Uses of Glucocorticoids

The diseases in which GCs have been shown to have a pronounced anti-inflammatory effect include inflammatory arthritides such as rheumatoid arthritis, ankylosing spondylitis and psoriatic arthropathy, other rheumatoid diseases such as systemic lupus erythematosis, sderoderma, vascutitides including temporal arteritis and polyarteritis nodosa, inflammatory bowel disease such as Crohns disease and ulcerative colitis, lung diseases such as asthma and chronic obstructive airways disease, as well as many other conditions such as polymyalgia rheumatica. GCs have also been used very extensively for their immunosuppressive properties in the prevention and treatment of transplant rejection. Finally GCs have been used for their anti-tumour effects in a number of malignancies. The activity of GCs is in the treatment of lymphoproliferative and other malignances is thought to be due to the ability of GCs to induce apoptosis (McColl K S, He H, Zhong H, Whitacre C M, Berger N A, Distelhorst C W., Mol. Cell Endocrinol. 1998; 139: 229-38; MiyashitaT, Nagao K, Krajewski S, Salvesen G S, Reed J C, Inoue T et al, Cell Death. Differ. 1998; 5: 1034-41).

The use of steroidal GCs, particularly in inflammatory disease, has been severely limited by their side effects, as discussed below. A number of approaches have been taken to overcome the side effects of the drugs. The most frequently adopted approach has been to apply the steroid locally to the site of inflammation. Target organs where this approach has been adopted include the lung with oral inhalation for the treatment of asthma and chronic obstructive airways disease; the nose with local installation for the treatment of allergic rhinitis; the eye with local installation for the treatment of a number of serious inflammatory eye conditions such as uveitis; in large joints with intra-articuiar injection of steroids to treat inflammation ; and on the skin for the treatment of eczema, psoriasis and a range of other conditions of the skin. Local delivery has allowed dose reduction with a consequent reduction in systemic side effects.

Systemic side effects have been reduced further by the introduction over recent years of so-called “soft steroids” such as fluticasone, for topical application. These soft steroids are inactivated rapidly by metabolism following absorption into the systemic circulation thus minimising systemic side effects. Local application of soft steroids, however, is still associated with significant local side effects such as skin thinning. Soft steroids are of no use when systemic administration of the drug is required in diseases such as temporal arteritis or polymyalgia rheumatic, and there are numerous reports of unwanted systemic effects resulting from use of these agents.

The side effects of steroids include the following: osteoporosis; growth impairment; vascular osteonecrosis; proximal myopathy; impaired glucose tolerance or frank diabetes; fluid retention and oedema; hypertension; hypokalaemia; Cushingoid fades; weight gain; obesity; euphoria; psychosis; insomnia; raised intracranial pressure; aggravation of epilepsy; memory impairment; hippocampal atrophy; peptic ulceration; pancreatitis; suppression of the hypothalamic pituitary axis; raised introcular pressure; glaucoma; papiloedema; skin thinning; reduced resistance to infection; impaired wound healing.

Despite their catalogue of side effects steroids are still used very widely because their anti-inflammatory effects exceed those of any other drug class. They continue to have a central role in the treatment and prevention of transplant rejection and the treatment of lymphoproliferative disorders and certain other malignancies.

There is a continuing need for novel steroids with the same efficacy as the existing drugs in this class but with a reduced side effect potential.

Attempts to identify novel, synthetic GR ligands capable of mimicking the effects seen with mutated GR protein have met with some success. However, previously described ligands or molecules do not have a sufficiently wide dissociation of the two mechanisms of GR action to be of use therapeutically.

One early candidate molecule, RU24858, showed promise in-vitro, but in further analyses was revealed to be a low potency, low efficacy full agonist (Vayssiere BM, Dupont S, Choquart A, Petit F, Garcia T, Marchandeau C et al, Mol. Endocrinol. 1997; 11:1245-55). In addition RU24858 does not induce apoptosis and hence lacks one of the important activities of GCs, as well as activating the progesterone receptor, thereby having an undesirable lack of specificity of action.

Previous studies have tried to differentiate the effects of known and novel steroids in terms of ability to cause transactivation and transrepression (Vanden Berghe Wet al., Mol Pharmacol. 1999; 56: 797-806; Hofmann TG et al., FEBS Lett. 1998; 441:441-6)).

Subsequent iterations of drug design have revealed candidate molecules, but none are in trial for systemic application.

WO02/36606 discloses certain steroid derivatives of general formula:

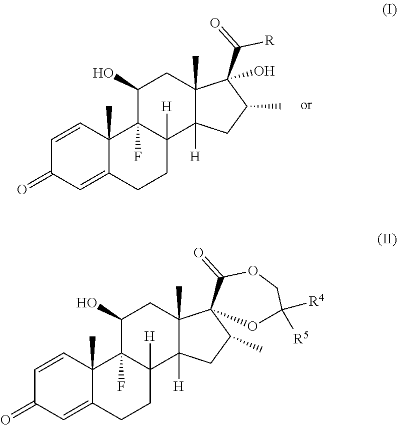

wherein R is —NH₂, —NHR¹, —NHOR², —NHNHR², —NHCOR², or —CH₂OC(O)NHR³ and R¹ is C₁₋₄alkyl, C₃₋₆cycloalkyl, C(Ph)ₙ where n is 1-3, R² is methyl or ethyl, R³ is alkyl, cycloalkyl, substituted alkyl, substituted cycloalkyl, aryl, heteroaryl,. substituted aryl, or substituted heteroaryl and R⁴ and R⁵ are C₁₋₄alkyl. The compounds were found to induce apoptosis in pro-inflammatory cells. Pro-drugs of certain steroid derivatives were also disclosed.

Notwithstanding the above, there remains a need for high affinity, high potency, GR ligands, which are selective for anti-NFkB activity and which have sufficient separation of the beneficial, anti-rheumatic and anti-inflammatory, actions from off-target effects on glucose metabolism and bone metabolism.

### SUMMARY OF THE INVENTION

One aspect of the invention pertains to certain steroid derivative compounds, as described herein. In particular, the invention provides steroid derivative compounds, of formula (I):

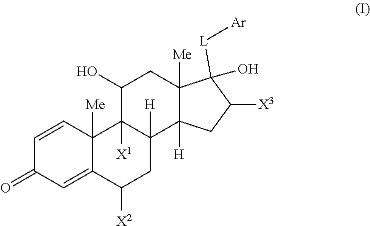

wherein X¹, X², X³ L, and Ar are as defined herein.

Another aspect of the invention pertains to compositions (e.g., a pharmaceutical compositions) comprising a steroid derivative compound as described herein, and a pharmaceutically acceptable carrier or diluent.

Another aspect of the invention pertains to methods of preparing a composition (e.g., a pharmaceutical composition) comprising the step of admixing a steroid derivative compound as described herein, and a pharmaceutically acceptable carrier or diluent.

Another aspect of the present invention pertains to methods of treatment comprising administering to a subject in need of treatment a therapeutically-effective amount of a steroid derivative compound, as described herein, preferably in the form of a pharmaceutical composition.

Another aspect of the present invention pertains to a steroid derivative compound as described herein for use in a method of treatment of the human or animal body by therapy.

Another aspect of the present invention pertains to use of a steroid derivative compound, as described herein, in the manufacture of a medicament for use in treatment.

In some embodiments the treatment comprises treating or preventing an inflammatory condition, treating haematological and other malignancies, causing immunosuppression or preventing or treating transplant rejection in a subject.

In some embodiments, treatment may be treatment of: rheumatoid arthritis, ankylosing spondylitis and psoriatic arthropathy, other rheumatoid diseases such as systemic lupus erythematosis, sderoderma, vasculitides including temporal arteritis and polyarteritis nodosa, inflammatory bowel disease such as Crohns disease and ulcerative colitis, lung diseases such as asthma and chronic obstructive airways disease, as well as many other conditions such as polymyalgia rheumatic.

Another aspect of the present invention pertains to a kit comprising (a) a steroid derivative compound as described herein, preferably provided as a pharmaceutical composition and in a suitable container and/or with suitable packaging; and (b) instructions for use, for example, written instructions on how to administer the compound.

Another aspect of the present invention pertains to certain methods of synthesis of steroid derivative compounds, as described herein.

As will be appreciated by one of skill in the art, features and preferred embodiments of one aspect of the invention will also pertain to other aspect of the invention.

### BRIEF DESCRIPTION OF THE FIGURES

FIG. 1 shows the results of head-to-head comparison between dexamethasone and an exemplary compound of the invention, Dex124.

FIG. 2 shows basal transactivation with Dex124 when transactivation reporter cells with a fixed concentration of Dex124 or vehicle were subjected to a dose-response of dexamethasone.

FIG. 3 shows the comparison of Dex124 with prednisolone and the cognate ligand for each of the progesterone receptor (PR), androgen receptor (AR), and mineralocorticoid receptor (MR).

FIG. 4 compares steroid-induced repression of pro-inflammatory cytokine expression by Dex124 and prednisolone.

FIG. 5 shows dose-dependent inhibition of rat adjuvant arthritis with Dex124, and comparison with prednisolone.

FIG. 6 show the evolution of arthritis in response to prednisolone or Dex124.

FIG. 7 summarises head-to-head comparison data in the clinical arthritis model.

FIG. 8 shows induction of serum glucose in response to dexamethasone, prednisolone and Dex124.

FIG. 9 shows dose-dependent inhibition of osteocalcin concentration with prednisolone, and a right-shifted dose response for Dex124.

FIG. 10 shows the dose-dependent decrease in bone increment for prednisolone and Dex124.

FIG. 11 is the CT reconstruction of the long bone.

FIG. 12 shows results from microcomputer tomographic imaging of bone structure and mineral content.

FIG. 13 shows the epiphysis, or end, of the long bone which consists of medial and lateral condyles.

### DETAILED DESCRIPTION AND PREFERENCES

Compounds

Described herein are compounds of general formula (I):

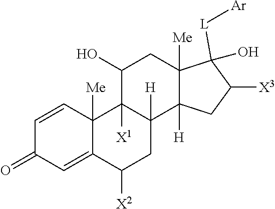

wherein:

X¹ and X² are each independently selected from —H and —F;

X³ is selected from —H and -Me;

L is a linker group selected from L¹ and L²; and

Ar is selected from phenyl and C₅₋₉heteroaryl, optionally substituted with one or more substituents R^{A};

wherein:

each R^{A} is independently selected from: —F, —Cl, —Br, —I, —OR^{O}, —N(R^{N})₂, —C(=O)OR^{O}, —C(═O)N(R^{N})₂, —SO₂N(R^{N})₂, —CF₃, and —CN, wherein each R^{O} and RN is independently selected from —H and —C₁₋₄ alkyl;

L1 is:

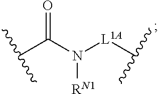

L2 is:

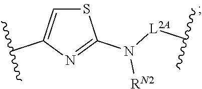

L^{1A} is selected from -L^{A}- and -L^{B}-O-;

L^{2A} is selected from —C(═O)—, —C(═O)-L^{B}-, and -L^{B}-;

wherein L^{A} is saturated C₃₋₄ alkylene and L^{B} is saturated C₁₀₄ alkylene;

and R^{N1} and R^{N2} are each independently selected from —H and -Me.

In a preferred embodiment, the invention provides compounds of formula (II):

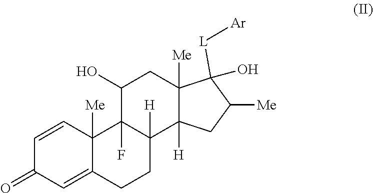

wherein L and Ar are as previously defined.

In some embodiments, the compound is a compound of formula (IIa):

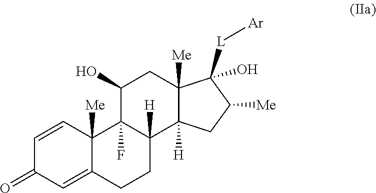

wherein L and Ar are as defined above.

In some embodiments, the compound is a compound of formula (I) or (Ia), as defined herein, with the proviso that the compound is not 9-fluoro-11,17-dihydroxy-N-(3-imidazol-1-ylpropyl)-3-oxo-6,7,8,10,11,12,13,14,15,16-decahydrocyclopenta[a]phenanthrene-17-carboxamide.

The Groups X¹ and X²

In the compounds of formula (I) and (Ia), X¹ and X² are each independently selected from —H and —F. Some embodiments of the invention include the following:

(X1-1) A compound of formula (I) or (Ia) as defined herein, wherein X¹ and X² are both independently —H.

(X1-2) A compound of formula (I) or (Ia) as defined herein, wherein X¹ is independently —H and X2 is independently —F.

(X1-3) A compound of formula (I) or (Ia) as defined herein, wherein X¹ is independently —F and X2 is independently —H.

(X1-4) A compound of formula (I) or (Ia) as defined herein, wherein X¹ and X² are both independently —F.

The Group X³

In the compounds of formula (I) and (Ia), X³ is independently selected from —H and -Me. Some embodiments of the invention include the following:

(X1-5) A compound according to any one of paragraphs (X1-1) to (X1-4) wherein X³ is independently —H.

(X1-6) A compound according to any one of paragraphs (X1-1) to (X1-4) wherein X³ is independently -Me.

The Group Ar

In the compounds of formula (I), (Ia), (II) and (IIa) Ar is independently selected from phenyl and C₅₋₉ heteroaryl, optionally substituted with one or more substituents R^{A}.

The term “C₅₋₉heteroaryl”, as used herein, pertains to a monovalent moiety obtained by removing a hydrogen atom from a ring atom of a C₅₋₉heteroaromatic compound, said compound having one ring or two rings (i.e., fused) and having from 5 to 9 ring atoms, wherein at least one of said ring(s) is an aromatic ring, and wherein “C₅₋₉” denotes ring atoms, whether carbon atoms or heteroatoms.

Some embodiments of the invention include the following:

(A1-2) A compound according to any one of paragraphs (X1-1) to (X1-6) wherein Ar is independently selected from phenyl, furanyl, thienyl, pyrrolyl, thiazolyl, isothiazolyl, oxazolyl, isoxazolyl, imidazolyl, pyrazolyl, triazolyl, tetrazolyl, pyridinyl, pyrimidinyl, pyridazinyl, and pyrazinyl, indolyl, benzofuranyl, benzoxazolyl, benzimidazolyl, benzothienyl, and benzothiazolyl, optionally substituted with one or more substituents R^{A}.

(A1-3) A compound according to any one of paragraphs (X1-1) to (X1-6) wherein Ar is independently selected from phenyl, pyridinyl, thiazolyl, pyrrolyl, furanyl, benzothiazolyl, and benzoxazolyl, optionally substituted with one or more substituents R^{A}.

(A1-4) A compound according to any one of paragraphs (X1-1) to (X1-6) wherein Ar is independently phenyl, optionally substituted with one or more substituents R^{A}.

(A1-5) A compound according to any one of paragraphs (X1-1) to (X1-6) wherein Ar is independently pyridinyl, optionally substituted with one or more substituents R^{A}. (A1-6) A compound according to any one of paragraphs (X1-1) to (X1-6) wherein Ar is independently 2-pyridinyl, 3-pyridinyl or 4-pyridinyl, optionally substituted with one or more substituents R^{A}.

(A1-7) A compound according to any one of paragraphs (X1-1) to (X1-6) wherein Ar is independently 2-pyridinyl or 4-pyridinyl, optionally substituted with one or more substituents R^{A}.

(A1-8) A compound according to any one of paragraphs (X1-1) to (X1-6) wherein Ar is independently 2-pyridinyl, optionally substituted with one or more substituents R^{A}.

(A1-9) A compound according to any one of paragraphs (X1-1) to (X1-6) wherein Ar is independently 4-pyridinyl, optionally substituted with one or more substituents R^{A}.

(A1-10) A compound according to any one of paragraphs (A1-1) to (A1-9), wherein Ar is unsubstituted.

(A1-11) A compound according to any one of paragraphs (A1-1) to (A1-9), wherein Ar is substituted by one or more substituents R^{A}.

(A 1-12) A compound according to any one of paragraphs (A1-1) to (A1-9), wherein Ar is substituted by one or two R^{A} groups.

(A1-13) A compound according to any one of paragraphs (A1-1) to (A1-9), wherein Ar is substituted by one R^{A} group.

(A1-14) A compound according to any one of paragraphs (A1-1) to (A1-9), wherein Ar is substituted by two R^{A} groups.

The group Ar, where present, is optionally substituted by one or more substituents R^{A}. Some embodiments of the invention include the following:

(R1-1) A compound according to any one of paragraphs (A 1-11) to (A1-14), wherein each R^{A} is independently selected from —F, —Cl, —Br, —I, —OR^{O}, —N(R^{N})₂, —C(═O)OR^{O}, —C(═O)N(RN)₂, —SO₂N(RN)2, —CF₃, and —CN, wherein each R^{O} and R^{N} is independently selected from —H and —C₁₋₄ alkyl.

(R1-2) A compound according to any one of paragraphs (A 1-11) to (A1-14), wherein R^{A} is independently selected from —F, —OR^{O}, —C(═O)OR^{O}, and —CF₃, wherein each R^{O} is independently selected from —H and —C₁₋₄ alkyl.

(R1-3) A compound according to (R1-1) or (R1-2) wherein each R^{O} and R^{N-}, if present, is independently selected from —H, -Me, -Et, -nPr, -iPr, -nBu, -iBu, and -tBu.

(R1-4) A compound according to (R1-1) or (R1-2) wherein each R^{O} and R^{N}, if present, is independently selected from —H, -Me, and -Et.

(R1-5) A compound according to (R1-1) or (R1-2) wherein each R^{O} and R^{N}, if present, is independently —H or -Me.

(R1-6) A compound according to (R1-1) or (R1-2) wherein each R^{O} and R^{N}, if present, is independently —H.

(R1-7) A compound according to any one of paragraphs (A 1-11) to (A1-14), wherein, R^{A} is independently selected from —F, —OH, —OMe, —CO₂Me, —CO₂H, and —CF₃.

(R1-8) A compound according to any one of paragraphs (A1-11) to (A1-14), wherein R^{A} is independently selected from —F, —OMe, and —CO₂Me.

(R1-9) A compound according to any one of paragraphs (A1-11) to (A1-14), wherein R^{A} is independently —F or —Cl.

(R1-10) A compound according to any one of paragraphs (A1-11) to (A1-14), wherein R^{A} is independently —F.

(R1-11) A compound according to any one of paragraphs (A1-11) to (A1-14), wherein R^{A} is independently —OMe.

The Group L

In the compounds of formula (I), (Ia), (II) and (IIa), L is a linker group selected from L¹ and L², wherein:

L¹ is:

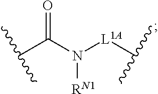

L² is:

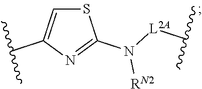

Some embodiments of the invention include the following:

(L1-1) A compound according to any one of paragraphs (A1-1) to (A1-14) and (R1-1) to (R1-11), wherein L is L¹.

(L1-2) A compound according to paragraph (L1-1) wherein L^{1A} is -L^{A}-, wherein L^{A} is independently saturated C₃₋₄ alkylene.

(L1-3) A compound according to paragraph (L1-2) wherein -L^{A}- is independently selected from:

—CH(CH₃)CH₂—; —CH₂CH(CH₃)—

—CH₂CH₂CH₂—;

—CH(CH₃)CH₂CH₂—; —CH₂CH(CH₃)CH₂—; —CH₂CH₂CH(CH₃)—; and —CH₂CH₂CH₂CH₂—.

(L1-4) A compound according to paragraph (L1-2) wherein -L^{A}- is —CH₂CH₂CH₂—or —CH₂CH₂CH₂CH₂—.

(L1-5) A compound according to paragraph (L1-2) wherein -L^{A}- is —CH2CH₂CH₂—.

(L1-6) A compound according to paragraph (L1-1) wherein L^{1A} is -L^{B1}-O—, wherein -L^{B}- is independently saturated C₁₋₄ alkylene.

(L1-7) A compound according to paragraph (L1-6), wherein L^{B} is independently selected from:

—CH₂—;

—CH(CH₃)—;

—CH₂CH₂—;

—CH(CH₃)CH₂—; —CH₂CH(CH₃)—

—CH₂CH₂CH₂—;

—CH(CH₃)CH₂CH₂—; —CH₂CH(CH₃)CH₂—; —CH₂CH₂CH(CH₃)—; and

—CH₂CH₂CH₂CH₂—.

(L1-8) A compound according to paragraph (L1-6), wherein L^{B} is —CH₂CH ₂—or —CH₂CH₂CH₂—.

(L1-9) A compound according to paragraph (L1-6), wherein L^{B} is —CH₂CH₂—.

(L1-10) A compound according to paragraph (L1-6), wherein L^{B} is —CH₂CH₂CH₂—.

(L1-11) A compound according to any of paragraphs (L1-1) to (L1-10), wherein R^{N1} is -Me.

(L1-12) A compound according to any of paragraphs (L1-1) to (L1-10), wherein R^{N1} is —H.

(L2-1) A compound according to any one of paragraphs (A1-1) to (A1-14) and (R1-1) to (R1-11), wherein Lis L².

(L2-2) A compound according to paragraph (L2-1) wherein L^{2A} is —C(═O)—.

(L2-3) A compound according to paragraph (L2-1) wherein L^{2A} is selected from —C(═O)-L^{B}- and -L^{B}-, wherein -L^{B}- is independently saturated C₁₋₄ alkylene.

(L2-4) A compound according to paragraph (L2-1) wherein L^{2A} is —C(═O)-L^{B}-, wherein -L^{B}- is independently saturated C₁₋₄ alkylene.

(L2-5) A compound according to paragraph (L2-1) wherein L^{2A} is -L^{B}-, wherein -L^{B}- is independently saturated C₁₋₄ alkylene.

(L2-6) A compound according to any of paragraphs (L2-3) to (L2-6), wherein L^{B} is independently selected from:

—CH₂—;

—CH(CH₃)—;

—CH₂CH₂—;

—CH(CH₃)CH₂—; —CH₂CH(CH₃)—

—CH₂CH₂CH₂—;

—CH(CH₃)CH₂CH₂—; —CH₂CH(CH₃)CH₂—; —CH₂CH₂CH(CH₃)—; and

—CH₂CH₂CH₂CH₂—.

(L2-7) A compound according to any of paragraphs (L2-3) to (L2-6), wherein L^{B} is —CH₂CH₂— or —CH₂CH₂CH₂—.

(L2-8) A compound according to any of paragraphs (L2-3) to (L2-6), wherein L^{B} is —CH₂CH₂—.

(L2-9) A compound according to any of paragraphs (L2-3) to (L2-6), wherein L^{B} is —CH₂CH₂CH₂—.

(L2-10) A compound according to any one of paragraphs (L2-1) to (L2-9) wherein R^{N2} is -Me.

(L2-11) A compound according to any one of paragraphs (L2-1) to (L2-9) wherein R^{N2} is —H.

### Certain Preferred Embodiments

In some embodiments, the compound is selected from the compounds listed in the following table and pharmaceutically acceptable salts, solvates and hydrates thereof:

| # | Structure |
|---|---|
| 1 | 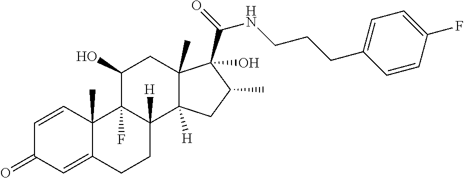 |
| 2 | 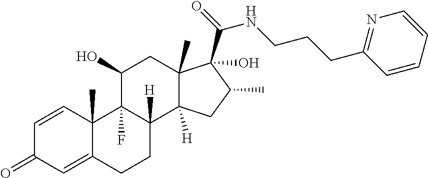 |
| 3 | 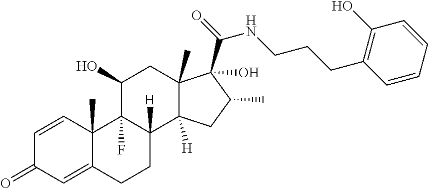 |
| 4 | 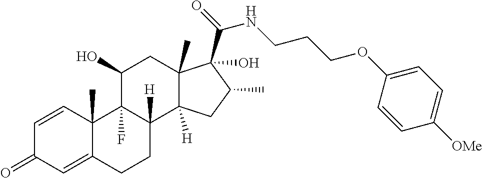 |
| 5 | 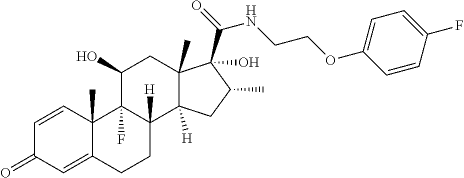 |
| 6 | 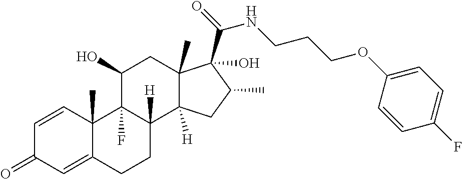 |
| 7 | 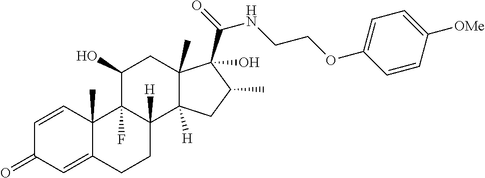 |
| 8 | 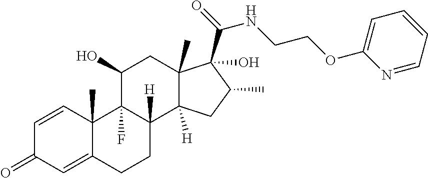 |
| 9 | 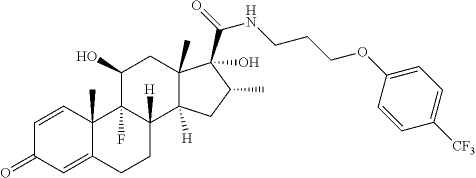 |
| 10 | 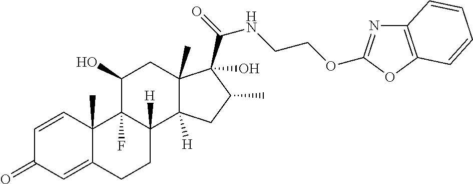 |
| 11 | 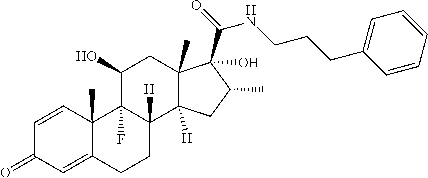 |
| 12 | 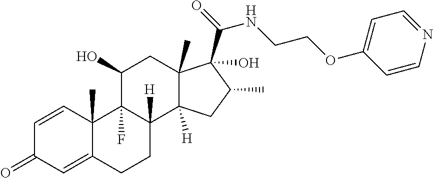 |
| 13 | 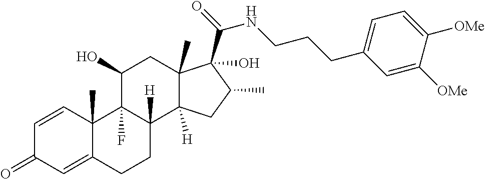 |
| 14 | 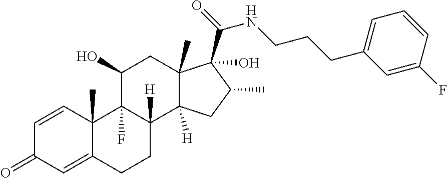 |
| 15 | 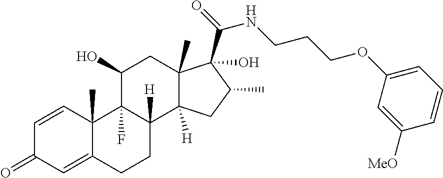 |
| 16 | 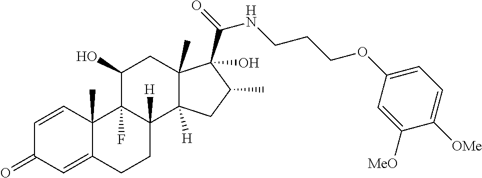 |
| 17 | 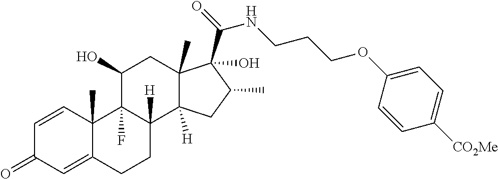 |
| 18 | 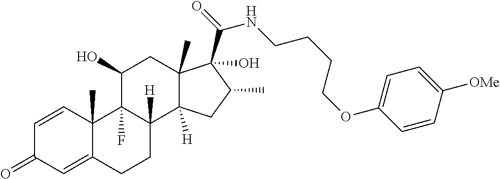 |
| 19 | 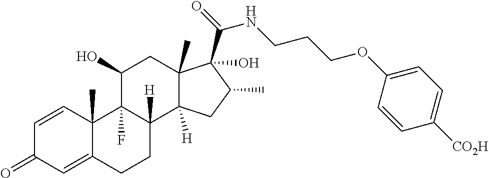 |
| 20 | 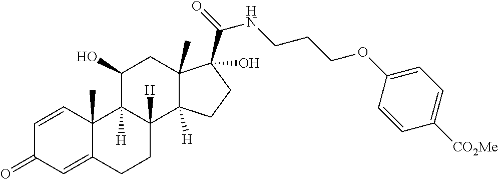 |
| 21 | 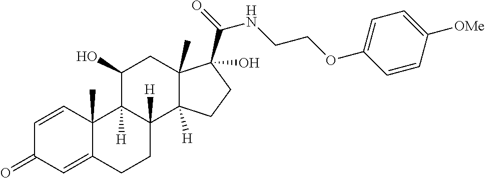 |
| 22 | 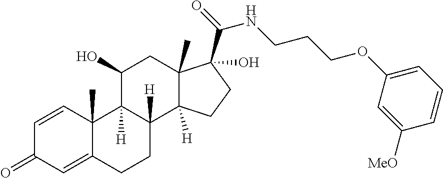 |
| 23 | 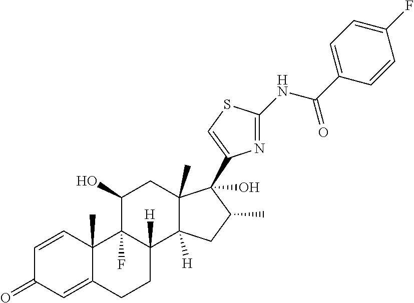 |
| 24 | 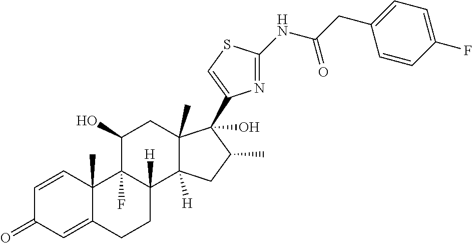 |
| 25 | 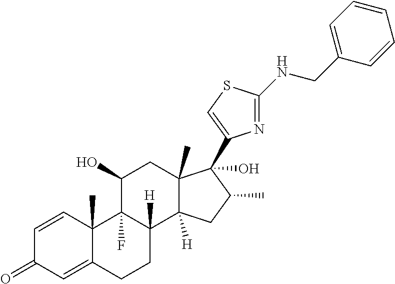 |
| 26 | 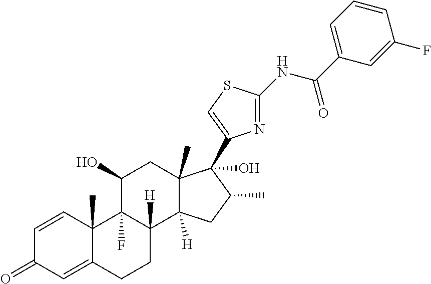 |
| 27 | 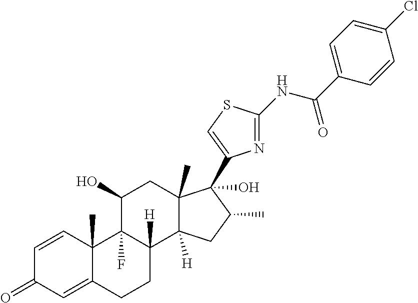 |
| 28 | 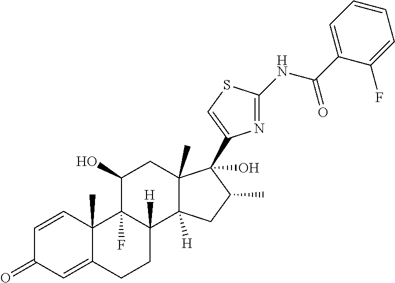 |
| 29 | 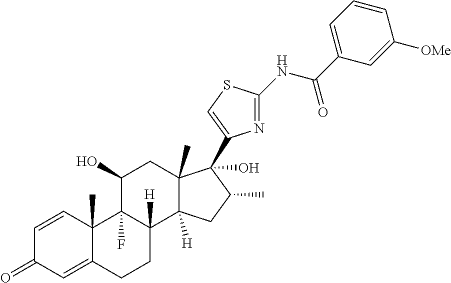 |
| 30 | 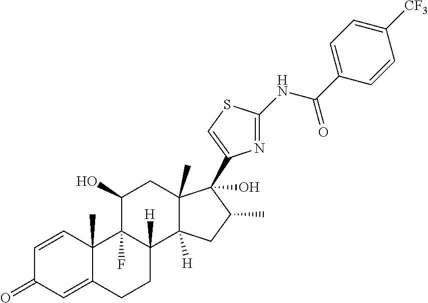 |
| 31 | 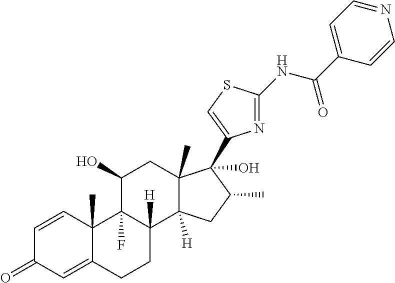 |
| 32 | 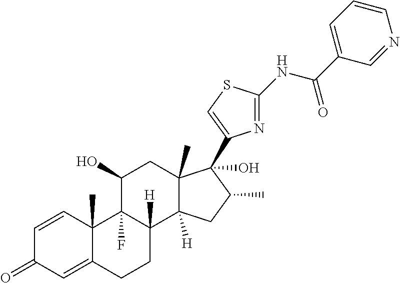 |
| 33 | 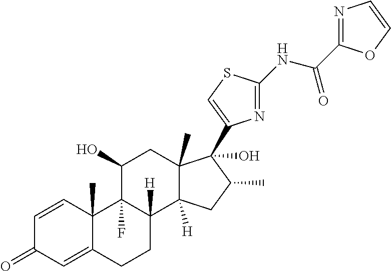 |
| 34 | 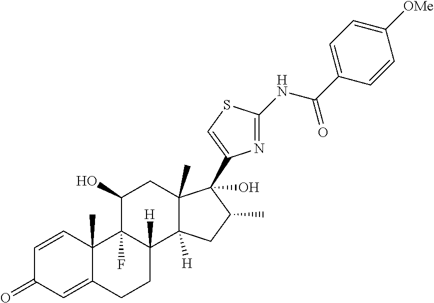 |
| 35 | 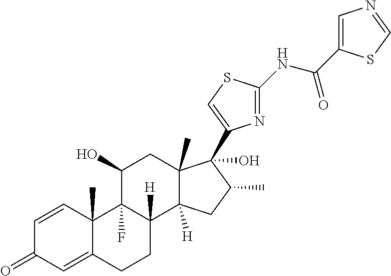 |
| 36 | 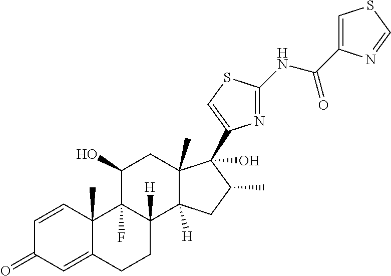 |
| 37 | 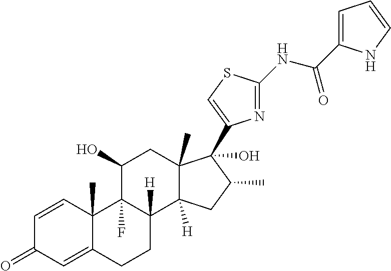 |
| 38 | 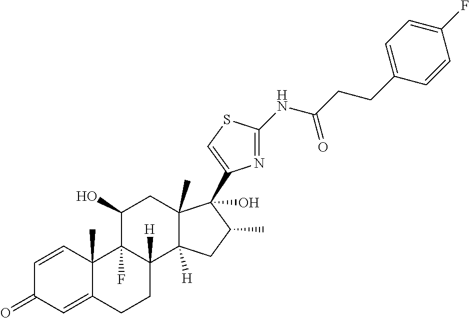 |
| 39 | 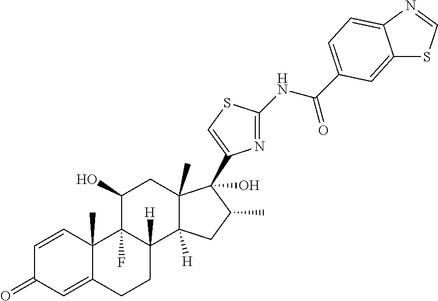 |
| 40 | 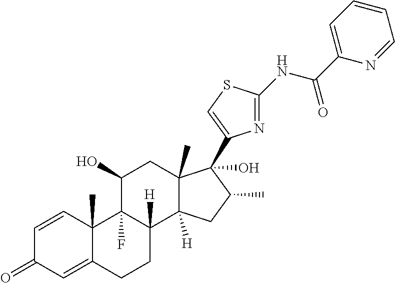 |
| 41 | 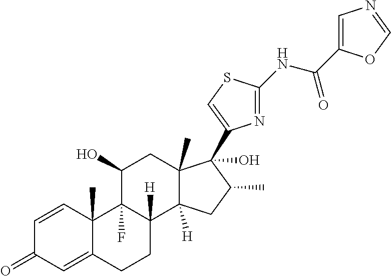 |
| 42 | 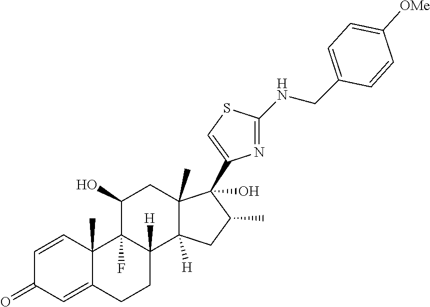 |
| 43 | 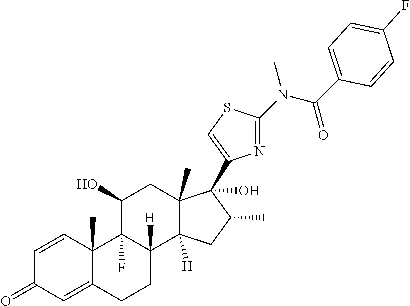 |
| 44 | 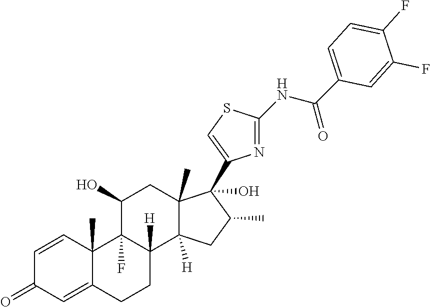 |
| 45 | 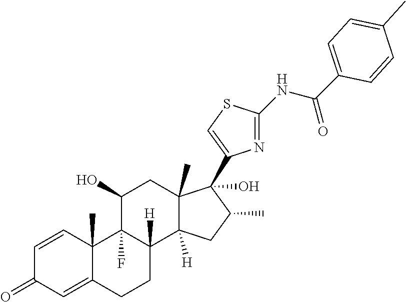 |
| 46 | 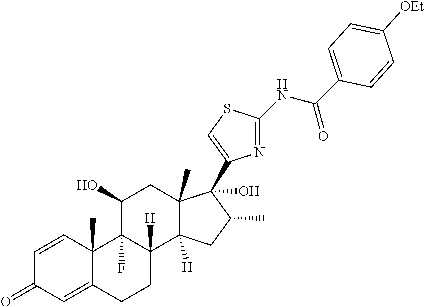 |
| 47 | 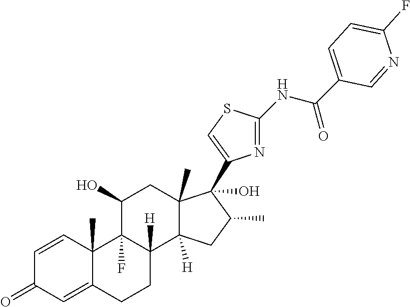 |

Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and p-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

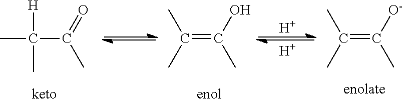

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Salts

It may be convenient or desirable to prepare, purify, and/or handle a corresponding salt of the compound, for example, a pharmaceutically-acceptable salt. Examples of pharmaceutical acceptable salts are discussed in Berge et al., 1977, “Pharmaceutically Acceptable Salts,” J. Pharm. Sci. Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

Hydrates and Solvates

It may be convenient or desirable to prepare, purify, and/or handle a corresponding hydrate or solvate of the compound (e.g., pharmaceutically acceptable hydrates or solvates of the compound). The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes hydrate and solvate forms thereof.

Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), ortrityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

For example, a carbonyl group may be protected as an oxime (—C(═NOH)—) or a substituted oxime (—C(═NOR)—), for example, where R is saturated aliphatic C₁₋₄alkyl.

Prodrugs

It may be convenient or desirable to prepare, purify, and/or handle the compound in the form of a prodrug. The term “prodrug,” as used herein, pertains to a compound which, when metabolised (e.g., in vivo), yields the desired active compound. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

General Synthetic Methods

Compounds of formula (I) may be prepared from commercially available starting materials using methods known in the art.

For example, compounds of general formula (I) wherein L is L¹ i.e. amide compounds of general formula:

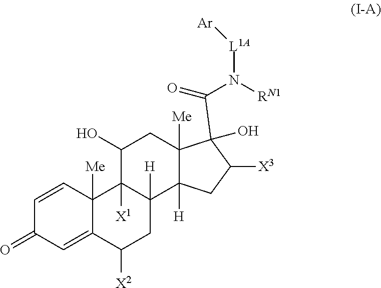

wherein X¹, X², X³, R^{N1}, L^{1A} and Ar are as defined above, may be prepared from a carboxylic acid compound of corresponding general formula (I-i):

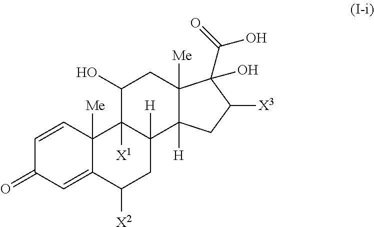

by coupling with an appropriate amine. The amine may be an amine of general formula:

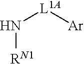

wherein R^{N1}, L^{1A} and Ar are as previously defined.

Coupling may comprise treatment of the acid of formula (II) with the amine and a suitable coupling agent. Suitable coupling agents include, but are not limited to, a carbonyl diimidazole, N,N′-dicyclohexylcarbodiimide, N,N′-diisopropylcarbodiimide, hydroxybenzotriazole (HOBt), benzotriazole-1-yl-oxy-tris-(dimethylamino)-phosphonium hexafluorophosphate (BOP) and benzotriazol-1-yl-oxytripyrrolidinophosphonium hexafluorophosphate (PyBOP).

The amine may be in the form of a salt, in which case a base (e.g. triethylamine) may be added to the coupling reaction.

Compounds of general formula (I-i) may be prepared from compounds of general formula (I-ii):

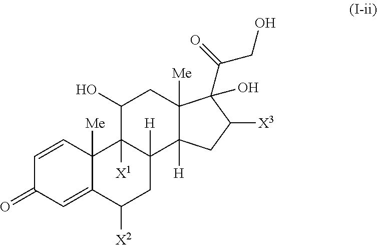

wherein X¹, X² and X³ are as defined above, by oxidation e.g. with sodium periodate.

Compounds of general formula (I-ii) are known e.g. dexamethasone, flumethasone, and prednisolone.

Compounds of general formula (I) wherein L is L² i.e. aminothiazole compounds of general formula:

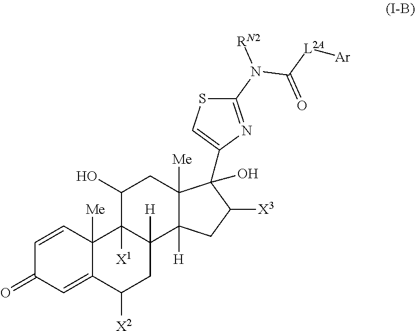

wherein X¹, X², X³, R^{N2}, L^{2A} and Ar are as defined above, may be prepared by ‘capping’ a thiazole compound of corresponding general formula (I-iii):

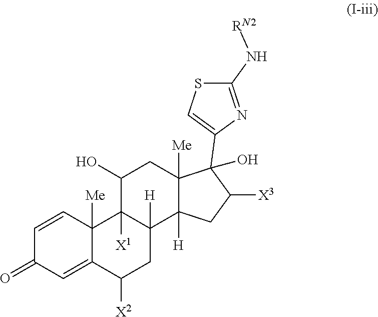

with an appropriate carboxylic acid (or corresponding activated carboxylic acid derivative, such as an acid chloride). The acid may be of general formula:

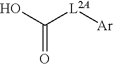

wherein L^{2A} and Ar are as previously defined.

The thiazole compound of general formula (I-iii) can be prepared may be prepared from compounds of general formula (I-ii):

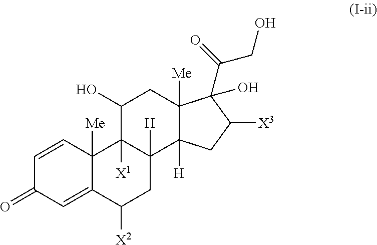

as defined above, for example by reaction with methanesulfonyl chloride followed by thiourea.

### Medical Uses/Methods of Treatment

The steroid derivative compounds described herein are of use in methods of medical treatment.

The steroid derivative compounds described herein find use, for example, in the treatment of inflammatory conditions, haematological and other malignancies, in causing immunosuppression, and in preventing or treating transplant rejection. The compounds described herein are also useful in methods for inducing apoptosis in target cells.

In some embodiments, the compounds are useful to induce apoptosis in pro-inflammatory cells and/or malignant cells.

In some embodiments treatment comprises treatment or prevention of inflammatory conditions including inflammatory arthritides such as rheumatoid arthritis, ankylosing spondylitis and psoriatic arthropathy, other rheumatoid diseases such as systemic lupus erythematosis, sderoderma, vasculitides including temporal arteritis and polyarteritis nodosa, inflammatory bowel disease such as Crohns disease and ulcerative colitis, lung diseases such as asthma and chronic obstructive airways disease, as well as many other conditions such as polymyalgia rheumatica.

In some embodiments, treatment comprises treatment or prevention of tumour, haematological malignances, lymphoproliferative malignances and other malignances.

In some embodiments, treatment comprises prevention or treatment of transplant rejection.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, molecularly-targeted agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

Routes of Administration

The compound or pharmaceutical composition comprising the compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

The Subject/Patient

The subject/patient may be a chordate, a vertebrate, a mammal, a placental mammal, a marsupial (e.g., kangaroo, wombat), a monotreme (e.g., platypus) a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

Formulations

While it may be possible for the steroid derivative compound to be administered alone, it is preferable to present it as a pharmaceutical formulation (e.g., composition, preparation, medicament) comprising at least one steroid derivative compound as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one steroid derivative compound as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, lozenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, lozenges, pastilles, as well as patches, adhesive plasters, depots, and reservoirs. Lozenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, lozenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, lozenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Dosage

It will be appreciated by one of skill in the art that appropriate dosages of the compounds, and compositions comprising the compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

### EXAMPLES

The following examples are provided solely to illustrate the present invention and are not intended to limit the scope of the invention, as described herein.

### Synthesis Examples

Abbreviations:

DCM=dichloromethane

THF=tetrahydrofuran

DMF=dimethylformamide

EtOH=ethanol

MeOH=methanol

EtOAc=ethyl acetate

DMSO=dimethyl sulfoxide

TEA=triethylamine

PyBOP=benzotriazol-1-yl-oxytripyrrolidinophosphonium hexafluorophosphate

EDC=1-ethyl-3-(3-dimethylaminopropyl)carbodiimide

HOBT=hydroxybenzotriazole

Boc=tert-butoxycarbonyl

DIPEA=N,N-diisopropylethylamine

ESI=electrospray ionisation

LCMS=liquid chromatography-mass spectrometry

UPLC-MS=ultra performance liquid chromatography-mass spectrometry

HRMS=high resolution mass spectrometry

NMR=nuclear magnetic resonance

TLC=thin layer chromatography

Materials and Methods:

All reagents were obtained commercially and used as received. All reactions were run under a nitrogen atmosphere. Reactions were monitored by TLC with detection using the appropriate staining reagent or by ESI-LCMS (positive ion mode with UV detection at 254 nm).

¹H NMR chemical shifts are reported in δ ppm using the residual solvent peak as the internal reference. Data for NMR spectra are reported as follows: chemical shift (δ ppm), multiplicity (s=singlet, br s=broad singlet, d=doublet, t=triplet, q=quartet, dd=doublet of doublets, m=multiplet), coupling constant (Hz), integration.

NMR Instrumentation:

NMR spectra were recorded on a Bruker DRX 500 MHz NMR or Bruker DPX 250MHz NMR (B114).

Configuration of the Bruker DRX 500 MHz NMR:

High performance digital NMR spectrometer, 2-channel console and Windows XP host workstation running Topspin version 1.3.

Equipped with:

Oxford instruments magnet 11.74 Tesla (500 MHz proton resonance frequency) B-VT 3000 temperature controller

GRASP II gradient spectroscopy accessory for fast acquisition of 2D pulse sequences

Deuterium lock switch for gradient shimming

5 mm Broad Band Inverse geometry double resonance probe with automated tuning and matching (BBI ATMA). Allows ¹H observation with pulsing/decoupling of nuclei in the frequency range ¹⁵N and ³¹P with ²H lock and shielded z-gradient coils.

Configuration of the Bruker DPX 250 MHz NMR (B114):

High performance one bay Bruker 250 MHz digital two channel NMR spectrometer console and Windows XP host workstation running XwinNMR version 3.5.

Equipped with:

Oxford instruments magnet 5.87 Tesla (250 MHz proton resonance frequency) B-VT 3300 variable temperature controller unit

Four nucleus (QNP) switchable probe for observation of ¹H, ¹³C, ¹⁹F and ³¹P with ²H lock LCMS instrumentation:

3 min LCMS:

Column: Waters Atlantis dC18, 2.1 mm×50 mm, 3μm column.

HPLC system capable of gradient elution with UV or diode array detection.

UV detection is typically performed at a selected wavelength or over a scan range.

MS detection is typically performed over a mass range to include target masses and other ions of interest.

LC Conditions:

Flow rate=1.0 ml/min

Column temperature=40° C

| Time (mins) | % B |
|---|---|
| 0 | 5 |
| 2.50 | 100 |
| 2.70 | 100 |
| 2.71 | 5 |

0.1% Formic acid in water—Mobile phase “A”

0.1% Formic acid in acetonitrile—Mobile phase “B”

7 min LCMS:

Column: Waters Atlantis dC18, 2.1 mm×100 mm, 3 μm column.

HPLC system capable of gradient elution with UV or diode array detection.

UV detection is typically performed at a selected wavelength or over a scan range.

MS detection is typically performed over a mass range to include target masses and other ions of interest.

LC Conditions:

Flow rate=0.6 ml/min

Column temperature=40° C

| Time (mins) | % B |
|---|---|
| 0.00 | 5 |
| 5.00 | 100 |
| 5.40 | 100 |
| 5.42 | 5 |

0.1% Formic acid in water—Mobile phase “A”

0.1% Formic acid in acetonitrile—Mobile phase “B”

uPLC-MS, 2 min Method:

Instrument UPLC system capable of gradient elution with UV or diode array detection.

Column Acquity UPLC BEH C18 2.1×50 mm , 1.7 micron.

Detection wavelength 215 nm (with scan in the region 210-400 nm).

Data is typically collected over a range m/z 150 to 800 with a cone and capillary voltage

sufficient to generate the molecular ion. ES+ve and −ve (Cone voltage: 30 V; Capillary

voltage: 3.0 KV).

LC conditions:

Flow rate 0.7 ml/min

Column temperature: Ambient

| Time (mins) | % B |
|---|---|
| 0.00 | 5 |
| 1.5 | 100 |
| 1.7 | 100 |
| 1.8 | 5 |

Mobile phase A: 0.1 % Formic Acid in HPLC Water: 0.1 % Formic Acid in Acetonitrile (90:10)

Mobile phase B: 0.1 % Formic Acid in Acetonitrile: 0.1 % Formic Acid in HPLC Water (90:10)

### Examples 1-19

Examples 1 to 19 were prepared according to the following general Scheme:

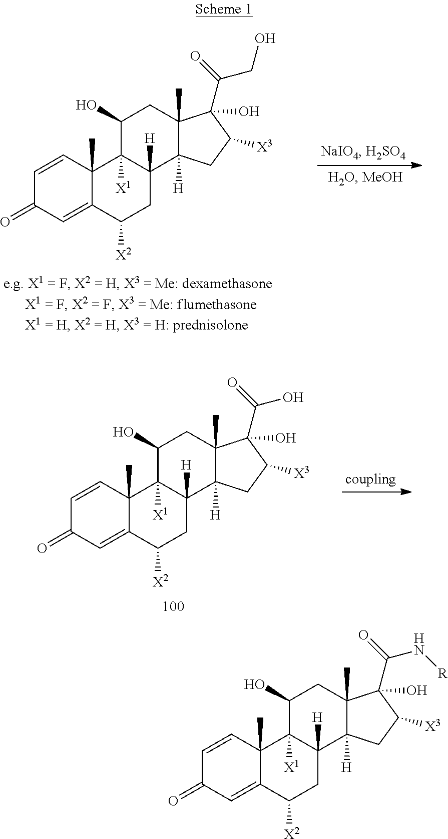

Synthesis of Carboxylic Acid 100

### 9-Fluoro-11,17-dihydroxy-10,13,16-trimethyl-3-oxo-6,7,8,9,10,11,12,13,14,15,16,17-dodecahydro-3H-cyclopenta[a]phenanthrene-17-carboxylic acid 100a

A single necked round bottom flask was charged with H₅IO₆ (1.12 eq) was added to a stirred suspension of dexamethasone (1 eq) in EtOH (10 vol) and water (4 vol). After 30 min, a clear solution resulted, which was stirred for an additional 5 h, forming a white solid. Progress of the reaction was monitored by TLC (TLC system: Dichloromethane: Methanol 95:05, R_{f}=0.1). To a reaction mixture water was added and stirring was continued for further 1 h. The suspension was filtered, and the solid was washed well with water and dried in vacuo to yield desired compound as a white solid (Yield 97%).UPLC-MS (2 min method): MH⁺ requires m/z=378 Found: m/z=379, Rt=1.06 min (97%) 1H NMR (400 MHz, DMSO) δ 12.42 (s, 1H), 7.29 (d, J=10.1 Hz, 1H), 6.22 (dd, J=10.1, 1.9 Hz, 1H), 6.00 (s, 1H), 5.24 (dd, J=3.8, 1.8 Hz, 1H), 4.63 (s, 1H), 4.22-4.07 (m, 1H), 2.82 (ddd, J=11.1, 7.2, 4.1 Hz,1H), 2.62 (td, J=13.6, 6.1 Hz, 1H), 2.41-2.24 (m, 2H), 2.06-1.94 (m, 2H), 1.81-1.72 (m, 1H), 1.62 (q, J=11.6 Hz, 1H), 1.55-1.46 (m, 4H), 1.34 (qd, J=12.7, 4.8 Hz, 1H), 1.05 (ddd, J=17.2, 8.8, 4.6 Hz, 1H),1.00 (s, 3H), 0.85 (d, J=7.1 Hz, 3H).

### 6,9-Difluoro-11,17-dihydroxy-10,13,16-trimethyl-3-oxo-6,7,8,9,10,11,12,13,14,15,16,17-dodecahydro-3H-cyclopenta[a]phenanthrene-17-carboxylic acid 100b

Flumethasone (205 mg; 0.5 mmol) was suspended in methanol (400 mL) and sodium periodate (161 mg; 0.75 mmol) water (300 mL) and 2 M sulphuric acid (100 mL) were added. After stirring at room temperature for 18 h, half of the methanol (210 mL) was removed by evaporation at reduced pressure and ice-cold water (800 mL) added. The suspension was stirred for 2 h then filtered, washed with water and dried. Yield=175 mg (88%). HRMS—C₂₁H₂₆F₂O₅—Expected mass: 397.1826, Found: 397.1823, Error=−0.8 ppm. ¹H NMR (500 MHz, DMSO-d6) δ_{H} ppm 0.86 (d, J=7.25 Hz, 3H) 0.99 (s, 3H) 1.09 (ddd, J=12.06, 8.20, 4.02 Hz, 1H) 1.39-1.47 (m, 1H) 1.49 (s, 3H) 1.54 (d, J=13.71 Hz, 1H) 1.65 (q, J=11.61Hz, 1H) 1.96-2.12 (m, 2H) 2.21 (dd, J=6.38, 4.97 Hz, 1H) 2.34-2.49 (m, 1H) 2.79-2.89(m, 1H) 4.09-4.18 (m, 1H) 4.69 (d, J=5.36 Hz, 1H) 5.33 (d, J=2.21 Hz, 1H) 5.53-5.72(m, 1H) 6.10 (s, 1H) 6.28 (dd, J=10.17, 1.66 Hz, 1H) 7.26 (d, J=10.25 Hz, 1H) 12.42 (br. s, 1H).

(1S,2R,10S,11S,14R,15S,17S)-14,17-dihydroxy-2,15-dimethyl-5-oxotetracyclo [8.1.0.0^{2,7}.0^{11,15}]heptadeca-3,6-diene-14-carboxylic acid 100c

Prednisolone (43 g; 0.119 mol) was suspended in methanol (860 mL) and water (645 mL). Sodium periodate (38 g; 0.179 mol) was added followed by 2M sulphuric acid (215 mL). The mixture was stirred at RT for 16 h then the bulk of the methanol was removed by rotary thin film evaporation before adding cold water (750 mL). The desired product was filtered off, washed with water and dried. Yield=40.7 g (98.8%). 1H NMR (500 MHz, DMSO-d6) δ_{H} ppm 0.84-0.88 (m, 1H) 0.90 (s, 3H) 0.99 (qd, J=13.14, 4.73 Hz, 1H) 1.27 (qd, J=11.72, 5.99 Hz, 1H) 1.39 (s, 3H) 1.44-1.57 (m, 2H) 1.57-1.76 (m, 3H) 1.94-2.07 (m, 2H) 2.29 (dd, J=13.08, 3.31 Hz, 1H) 2.41-2.48 (m, 1H) 2.52-2.58 (m, 1H) 4.22-4.30 (m, 1H) 4.64 (d, J=2.84 Hz, 1H) 4.81 (br. s., 1H) 5.91 (s, 1H) 6.16 (dd, J=10.09, 1.89 Hz, 1H) 7.32 (d, J=10.09 Hz, 1H) 12.26 (br. s., 1H).

General Method A:

Acid 100 (50 mg; 0.13 mmol) and PyBOP (76 mg; 0.15 mmol) were dissolved in DMF (0.5ml) and stirred at RT for 1-2 h. The amine (0.20 mmol-0.40 mmol) and, if the amine was a salt, triethylamine (0.20-0.40 mmol) were added and the reaction heated at 50-70° C. When complete, the mixture was cooled to RT then loaded onto a 2 g basic column and run through with acetonitrile (3 ml). This solution was then loaded onto a 2 g acid column and run with further acetonitrile. The solvents were evaporated to dryness and purified by chromatography on silica gel if required.

### Example 1

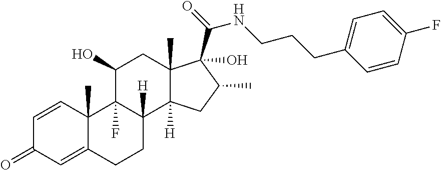

Synthesised by general method A using acid 100a and purified by flash chromatography with 25-75% EtOAc:heptanes. Yield=48 mg (72%), white solid. LCMS (7 minute method): 4.49 min (514; M+H). HRMS 514.2751 (expected for C₃₀H₃₇F₂NO₄ 514.2769).

¹H NMR (500 MHz, MeOD) δ ppm 7.41 (d, J=10.22 Hz, 1H) 7.18-7.25 (m, 2H) 6.95-7.02 (m, 2H) 6.28 (dd, J=10.15, 1.91 Hz, 1H) 6.08 (s, 1H) 4.23 (ddd, J=10.99, 3.81, 1.83 Hz, 1H) 3.26-3.34 (m, 1H) 3.08-3.20 (m, 2H) 2.67-2.77 (m, 1H) 2.61-2.67 (m, 2H) 2.35-2.51 (m, 2H) 2.17-2.25 (m, 2H) 1.85-1.92 (m, 1H) 1.79-1.85 (m, 2H) 1.71-1.79 (m, 1H) 1.59 (s, 3H) 1.49-1.57 (m, 1H) 1.47 (dd, J=13.89, 1.68 Hz, 1H) 1.16-1.22 (m, 1H) 1.10 (s, 3H) 0.89 (d, J=7.17 Hz, 3H).

### Example 2

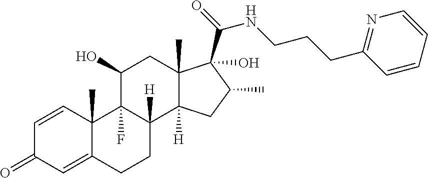

Synthesised by general method A using acid 100a and purified by flash chromatography with 100% EtOAc. Yield=58 mg (88%), white solid. LCMS (7 minute method): 2.97 min (497; M+H). HRMS 497.2797 (expected for C₂₉H₃₇FN₂O₄ 497.2816).

¹H NMR (500 MHz, MeOD) δ ppm 8.42-8.47 (m, 1H) 7.77 (td, J=7.67, 1.75 Hz, 1H) 7.42 (d, J=10.07 Hz, 1H) 7.35 (d, J=7.78 Hz, 1H) 7.23-7.28 (m, 1H) 6.28 (dd, J=10.07, 1.83 Hz, 1H) 6.08 (s, 1H) 4.20-4.28 (m, 1H) 3.32-3.38 (m, 1H) 3.08-3.25 (m, 2H) 2.79-2.88 (m, 2H) 2.67-2.77 (m, 1H) 2.35-2.52 (m, 2H) 2.16-2.27 (m, 2H) 1.85-1.96 (m, 3H) 1.70-1.80 (m, 1H) 1.59 (s, 3H) 1.47-1.57 (m, 2H) 1.17-1.22 (m, 1H) 1.10 (s, 3H) 0.90 (d, J=7.32 Hz, 3H).

### Example 3

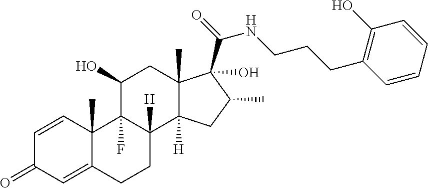

Synthesised by general method A using acid 100a and purified by flash chromatography with 75% EtOAc:heptanes. Yield=46 mg (78%), white solid. LCMS (7 minute method): 4.08 min (512; M+H). HRMS 512.2797 (expected for C₃₀H₃₈FNO₅ 512.2812)

¹H NMR (500 MHz, MeOD) δ ppm 7.41 (d, J=10.07 Hz, 1H) 7.05-7.10 (m, 1H) 6.99 (td, J=7.71, 1.68 Hz, 1H) 6.71-6.78 (m, 2H) 6.28 (dd, J=10.15, 1.91 Hz, 1H) 6.08 (s, 1H) 4.24 (ddd, J=11.02, 3.78, 1.83 Hz, 1H) 3.07-3.19 (m, 2H) 2.59-2.77 (m, 3H) 2.35-2.52 (m, 2H) 2.15-2.27 (m, 2H) 1.85-1.93 (m, 1H) 1.70-1.84 (m, 3H) 1.59 (s, 3H) 1.47-1.57 (m, 2H) 1.25-1.35 (m, 1H) 1.16-1.23 (m, 1H) 1.10 (s, 3H) 0.87-0.93 (m, 3H).

### Example 4

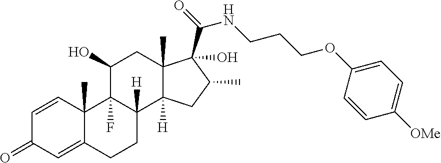

Purified by flash chromatography with 75% EtOAc:heptanes. Yield=53 mg (85%), white solid. LCMS (7 minute method): 4.27 min (542; M+H). HRMS 542.2909 (expected for C₃₁H₄₀FNO₆ 542.2918).

¹H NMR (500 MHz, MeOD) δ ppm 7.41 (d, J=10.07 Hz, 1H) 6.80-6.90 (m, 4 H) 6.28 (dd, J=10.07, 1.83 Hz, 1H) 6.08 (s, 1H) 4.19 (ddd, J=10.99, 3.81, 1.83 Hz, 1H) 3.98-4.03 (m, 2H) 3.73 (s, 3H) 3.43-3.50 (m, 1H) 3.33-3.39 (m, 1H) 3.13 (ddd, J=11.18, 7.21, 4.20 Hz, 1H) 2.67-2.77 (m, 1H) 2.36-2.51 (m, 2H) 2.15-2.24 (m, 2H) 1.94-2.02 (m, 2H) 1.85-1.92 (m, 1H) 1.70-1.80 (m, 1H) 1.59 (s, 3H) 1.52 (qd, J=12.92, 5.04 Hz, 1H) 1.45 (dd, J=13.96, 1.60 Hz, 1H) 1.20 (ddd, J=12.32, 8.28, 4.43 Hz, 1H) 1.09 (s, 3H) 0.89 (d, J=7.17 Hz, 3H).

### Example 5

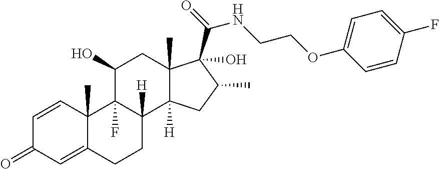

Synthesised by general method A using acid 100a and purified by flash chromatography with 30-60% EtOAc:heptanes. Yield=34 mg (51%), white solid. LCMS (7 minute method): 4.26 min (516; M+H). HRMS 516.2554 (expected for C₂₉H₃₅F₂NO₅ 516.2562).

¹H NMR (500 MHz, MeOD) δ ppm 7.41 (d, J=10.07 Hz, 1H) 6.97-7.03 (m, 2H) 6.90-6.97 (m, 2H) 6.28 (dd, J=10.07, 1.83 Hz, 1H) 6.07 (s, 1H) 4.22 (ddd, J=10.91, 3.81, 1.91 Hz, 1H) 3.98-4.07 (m, 2H) 3.66 (dt, J=13.89, 5.72 Hz, 1H) 3.50-3.58 (m, 1H) 3.08-3.18 (m, 1H) 2.66-2.77 (m, 1H) 2.35-2.51 (m, 2H) 2.15-2.25 (m, 2H) 1.84-1.92 (m, 1H) 1.71-1.81 (m, 1H) 1.43-1.62 (m, 5 H) 1.16-1.22 (m, 1H) 1.09 (s, 3H) 0.89 (d, J=7.17 Hz, 3H).

### Example 6

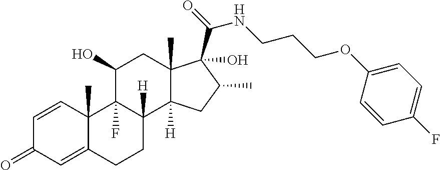

Synthesised by general method A using acid 100a and purified by flash chromatography with 30-60% EtOAc:heptanes. Yield=46 mg (67%), white solid. LCMS (7 minute method): 4.35 min (530; M+H) HRMS 530.2729 (expected for C₃₀H₃₇F₂NO₅ 530.2718)

¹H NMR (500 MHz, MeOD) δ ppm 7.41 (d, J=10.22 Hz, 1H) 6.95-7.02 (m, 2H) 6.88-6.95 (m, 2H) 6.28 (dd, J=10.15, 1.91 Hz, 1H) 6.08 (s, 1H) 4.21 (ddd, J=10.99, 3.74, 1.75 Hz, 1H) 3.97-4.06 (m, 2H) 3.41-3.50 (m, 1H) 3.36 (dt, J=13.54, 6.73 Hz, 1H) 3.07-3.18 (m, 1H) 2.66-2.77 (m, 1H) 2.34-2.51 (m, 2H) 2.14-2.25 (m, 2H) 1.93-2.03 (m, 2H) 1.83-1.92 (m, 1H) 1.69-1.80 (m, 1H) 1.42-1.63 (m, 5 H) 1.16-1.22 (m, 1H) 1.09 (s, 3H) 0.89 (d, J=7.32 Hz, 3H).

### Example 7

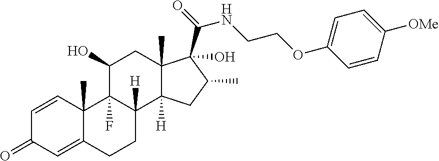

Synthesised by general method A using acid 100a and purified by flash chromatography with 50-75% EtOAc:heptanes. Yield=43 mg (63%), white solid. LCMS (7 minute method): 4.22 min (528; M+H). HRMS 528.2775 (expected for C₃₀H₃₈FNO₆ 528.2761).

¹H NMR (500 MHz, MeOD) δ ppm 7.41 (d, J=10.07 Hz, 1H) 6.86-6.91 (m, 2H) 6.81-6.86 (m, 2H) 6.28 (dd, J=10.07, 1.83 Hz, 1H) 6.07 (s, 1H) 4.22 (ddd, J=10.91, 3.66, 1.75 Hz, 1H) 3.95-4.05 (m, 2H) 3.74 (s, 3H) 3.64 (dt, J=13.85, 5.66 Hz, 1H) 3.52 (dt, J=13.89, 5.95 Hz, 1H) 3.13 (ddd, J=11.10, 7.21, 4.12 Hz, 1H) 2.66-2.77 (m, 1H) 2.35-2.52 (m, 2H) 2.14-2.25 (m, 2H) 1.85-1.92 (m, 1H) 1.71-1.81 (m, 1H) 1.43-1.61 (m, 5 H) 1.16-1.22 (m, 1H) 1.09 (s, 3H) 0.90 (d, J=7.32 Hz, 3H).

### Alternative Synthesis for Example 7

To a solution of carboxylic acid 100 (1.0 eq) in DCM (10 vol) was added TEA (3.0 eq) at room temperature, followed by EDC.HCI (1.2 eq.) and the reaction was stirred for 10 min. 4-methoxyphenoxyethylamine (1.2 eq) and HOBT (0.1 eq) were then added to the above mixture. The resultant mixture was stirred at room temperature for 16 h. The reaction mixture was diluted by DCM and washed it by sodium bicarbonate solution then dil HCl solution separate the organic layer dried over sodium sulphate and concentrated to yield a white solid. The residue was subjected to chromatographic separation using Silica gel and the title compound (Yield 78%) was obtained using DCM:Methanol (98:02) as eluant. LC-MS (3 min method): MH⁺ requires m/z=528 Found: m/z=527, Rt=2.83 min (97%). compound as a white solid. 1H NMR (400 MHz, DMSO) 1H NMR (300 MHz, DMSO) δ 0.79 (d, J=7.2 Hz, 3H), 0.94 (s, 3H), 1.12-0.99 (m, 1H), 1.46-1.21 (m, 2H), 1.48 (s, 3H), 1.61 (q, J=11.6 Hz, 1H), 1.85-1.69 (m, 1H), 2.14-1.94 (m, 2H), 2.44-2.18 (m, 2H),2.70-2.53 (m, 1H), 3.12-2.92 (m, 1H), 3.42-3.32 (m, 1H), 3.56-3.42 (m, 1H), 3.68 (s, 3H), 3.90 (t, J=6.2 Hz, 2H), 4.10 (d, J=10.6 Hz, 1H), 4.74 (s, 1H), 5.23 (d, J=3.0 Hz, 1H), 5.99 (s, 1H), 6.21 (dd, J=10.1, 1.6 Hz, 1H), 6.94-6.80 (m, 4H), 7.29 (d, J=10.1 Hz, 1H), 7.49 (t, J=5.7 Hz, 1H).

### Example 8

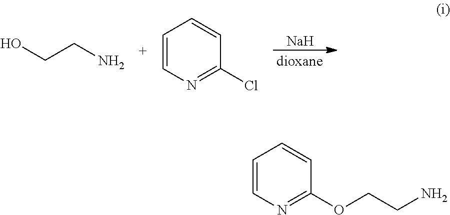

To NaH (60% in mineral oil, 2.5 mmol) in dioxane (10 mL) was added 2-hydroxyethylamine (2.5 mmol) and heated to reflux for 30 min. Reaction was cooled to RT, 2-chloropyridine was added and heated to 80° C for 18 h. Reaction was cooled to RT and concentrated. Water was added and then the mixture was extracted with DCM (×3). Combined organics were dried over Na₂SO₄, concentrated and purified by flash chromatography with 5-10% MeOH:DCM. Yield=211 mg (61%), pale yellow oil.

¹H NMR (500 MHz, CDCl₃) δ ppm 8.08-8.20 (m, 1H) 7.51-7.62 (m, 1H) 6.86 (dd, J=6.49, 5.72 Hz, 1H) 6.75 (d, J=8.24 Hz, 1H) 4.32 (t, J=5.34 Hz, 2H) 3.07 (t, J=5.26 Hz, 2H).

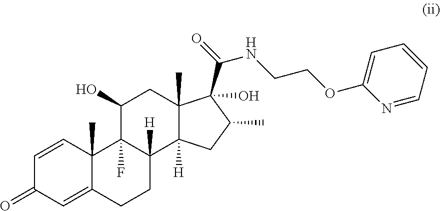

Synthesised by general method A using acid 100a and the amine prepared in step (i) and purified by flash chromatography with 50-70% EtOAc:heptanes. Yield=53 mg (82%), white solid. LCMS (7 minute method): 3.93 min (499; M+H). HRMS 499.2613 (expected for C₂₈H₃₅FN₂O₅ 499.2608).

¹H NMR (500 MHz, MeOD) δ ppm 8.12 (dd, J=4.73, 1.37 Hz, 1H) 7.65-7.73 (m, 1H) 7.41 (d, J=10.07 Hz, 1H) 6.96 (dd, J=6.48, 5.72 Hz, 1H) 6.83 (d, J=8.39 Hz, 1H) 6.28 (dd, J=10.07, 1.53 Hz, 1H) 6.08 (s, 1H) 4.32-4.43 (m, 2H) 4.20 (dd, J=10.91, 1.75 Hz, 1H) 3.69 (dt, J=13.92, 5.78 Hz, 1H) 3.53 (dt, J=13.92, 5.70 Hz, 1H) 3.07-3.16 (m, 1H) 2.72 (td, J=13.58, 5.95 Hz, 1H) 2.34-2.52 (m, 2H) 2.13-2.24 (m, 2H) 1.84-1.92 (m, 1H) 1.75 (q, J=11.75 Hz, 1H) 1.41-1.62 (m, 5 H) 1.16-1.22 (m, 1H) 1.08 (s, 3H) 0.89 (d, J=7.32 Hz, 3H).

### Example 9

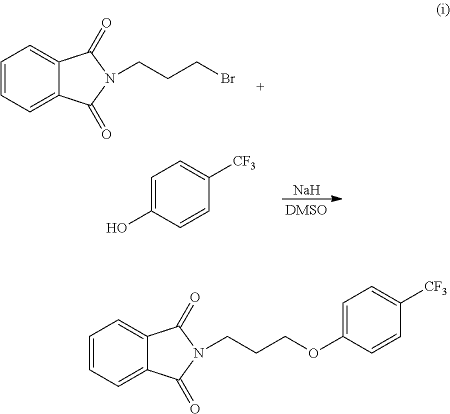

To NaH (60% in mineral oil, 5.0 mmol) was added DMSO (5 mL) at RT. Phenol (5.0 mmol) in DMSO (5 mL) was added slowly and stirred at RT for 2 h. Bromide (5.0 mmol) in DMSO (5 mL) was added and reaction stirred at RT for 16 h. Reaction poured into ice/water (100 mL) and precipitate collected by filtration. The precipitate was dissolved in DCM, dried over Na₂SO₄ and concentrated. The crude product was purified by flash chromatography with DCM. Yield=849 mg (57%), white solid

¹H NMR (500 MHz, CDCl₃) δ ppm 7.81-7.88 (m, 2H) 7.69-7.77 (m, 2H) 7.50 (d, J=8.70 Hz, 2H) 6.86 (d, J=8.70 Hz, 2H) 4.08 (t, J=5.95 Hz, 2H) 3.93 (t, J=6.79 Hz, 2H) 2.22 (quin, J=6.37 Hz, 2H).

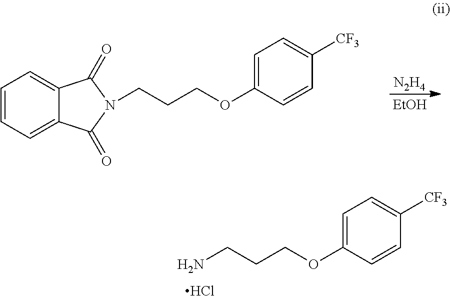 [SMILES: "EtOH" ; O=C1c2ccccc2C(=O)N1CCCOc1ccc(C(F)(F)F)cc1 ; "N2H4" ; NCCCOc1ccc(C(F)(F)F)cc1 ; Cl]

Phthalimide (1.69 mmol) and hydrazine hydrate (3.39 mmol) in EtOH (10 mL) was heated to reflux for 4 h. Reaction concentrated and partitioned between EtOAc and 5% aq. NaOH. Organics were separated and dried over Na₂SO₄. Filtered and 4N HCl in dioxane (2.5 mL) added. Stirred for 10 mins, concentrated and dried. Yield=381 mg (88%), off-white solid

¹H NMR (500 MHz, DMSO-d₆) δ ppm 8.18 (br. s., 3H) 7.66 (d, J=8.70 Hz, 2H) 7.13 (d, J=8.70 Hz, 2H) 4.16 (t, J=6.18 Hz, 2H) 2.87-3.00 (m, 2H) 2.06 (quin, J=6.75 Hz, 2H).

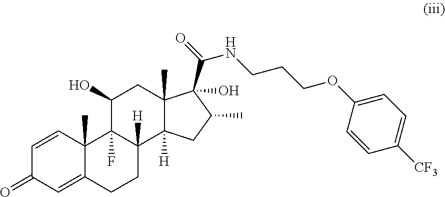

Synthesised by general method A using acid 100a and the amine prepared in step (ii) and purified by flash chromatography with 50% EtOAc:heptanes. Yield=33 mg (44%), white solid. LCMS (7 minute method): 4.66 min (580; M+H). HRMS 580.2675 (expected for C₃₁H₃₇F₄NO₅ 580.2686).

¹H NMR (500 MHz, MeOD) δ ppm 7.57 (d, J=8.70 Hz, 2H) 7.40 (d, J=10.22 Hz, 1H) 7.08 (d, J=8.70 Hz, 2H) 6.28 (dd, J=10.07, 1.53 Hz, 1H) 6.08 (s, 1H) 4.20 (dd, J=10.91, 1.60 Hz, 1H) 4.13 (t, J=6.10 Hz, 2H) 3.43-3.51 (m, 1H) 3.34-3.42 (m, 1H) 3.07-3.17 (m, 1H) 2.72 (td, J=13.62, 6.03 Hz, 1H) 2.35-2.51 (m, 2H) 2.15-2.25 (m, 2H) 1.98-2.09 (m, 2H) 1.84-1.93 (m, 1H) 1.75 (q, J=11.75 Hz, 1H) 1.41-1.61 (m, 5 H) 1.16-1.22 (m, 1H) 1.09 (s, 3H) 0.89 (d, J=7.32 Hz, 3H).

### Example 10

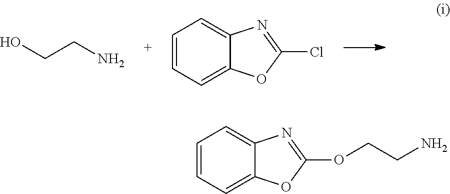

To NaH (60% in mineral oil, 2.5 mmol) in dioxane (10 mL) was added 2-hydroxyethylamine (2.5 mmol) and stirred at RT for 30 min. 2-Chlorobenzoxazole was added and heated to 80° C for 16 h. Reaction was cooled to RT and concentrated. Water was added and then the mixture was extracted with EtOAc (×3). Combined organics were washed with brine, dried over Na₂SO₄ and concentrated. Used crude in amide coupling.

LCMS (3 minute method): 1.01 min (179; M+H)

¹H NMR (500 MHz, MeOD) δ ppm 7.20-7.30 (m, 2H) 7.14 (td, J=7.61, 1.22 Hz, 1H) 6.95-7.07 (m, 1H) 3.67-3.81 (m, 2H) 3.43-3.56 (m, 2H).

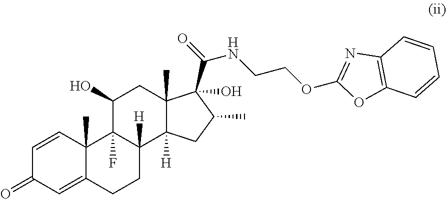

Synthesised by general method A using acid 100a and the amine prepared in step (i) and purified by flash chromatography with 60% EtOAc:heptanes and then by mass directed prep HPLC. Yield=15 mg (21%), white solid. LCMS (7 minute method): 4.26 min (539; M+H). HRMS 539.2548 (expected for C₃₀H₃₅FN₂O₆ 539.2557).

¹H NMR (500 MHz, MeOD) δ ppm 7.41 (d, J=8.09 Hz, 1H) 7.32-7.38 (m, 2H) 7.28 (t, J=7.55 Hz, 1H) 7.14-7.22 (m, 1H) 6.28 (d, J=10.07 Hz, 1H) 6.06 (s, 1H) 4.35-4.43 (m, 1H) 4.28-4.35 (m, 1H) 4.11 (d, J=10.22 Hz, 1H) 3.72-3.85 (m, 2H) 2.91-3.02 (m, 1H) 2.69 (td, J=13.50, 5.80 Hz, 1H) 2.31-2.47 (m, 2H) 1.99-2.15 (m, 2H) 1.85 (d, J=12.82Hz, 1H) 1.66 (q, J=11.80 Hz, 1H) 1.42-1.60 (m, 5 H) 1.16 (ddd, J=12.05, 8.16, 4.04 Hz, 1H) 1.04 (s, 3H) 0.88 (d, J=7.17 Hz, 3H)

### Example 11

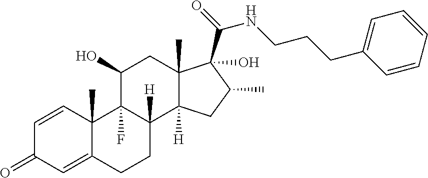

Synthesised by general method A using acid 100a and purified by flash chromatography with 50% EtOAc:heptanes. Yield=51 mg (79%), white solid. LCMS (7 minute method): 4.39 min (496; M+H). HRMS 496.2870 (expected for C₃₀H₃₈FNO₄ 496.2863).

¹H NMR (500 MHz, MeOD) δ ppm 7.41 (d, J=10.22 Hz, 1H) 7.23-7.28 (m, 2H) 7.18-7.23(m, 2H) 7.13-7.18 (m, 1H) 6.28 (dd, J=10.07, 1.68 Hz, 1H) 6.08 (s, 1H) 4.23 (dd, J=10.99, 1.68 Hz, 1H) 3.27-3.35 (m, 1H) 3.07-3.21 (m, 2H) 2.71 (td, J=13.54, 6.03 Hz, 1H) 2.65 (t, J=7.78 Hz, 2H) 2.35-2.51 (m, 2H) 2.15-2.24 (m, 2H) 1.80-1.92 (m, 3H) 1.75 (q, J=11.80 Hz, 1H) 1.58 (s, 3H) 1.44-1.57 (m, 2H) 1.16-1.23 (m, 1H) 1.10 (s, 3H) 0.90 (d, J=7.17 Hz, 3H).

### Example 12

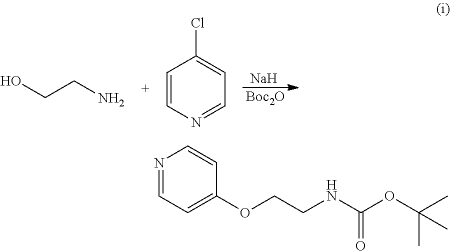

To NaH (60% in mineral oil, 12.0 mmol) in dioxane (10 mL) was added 2-aminoethanol (33.1 mmol) with cooling. Reaction was stirred for 1 h at RT and then 4-chloropyridine.HCI (4.0 mmol) was added and stirred at RT for 16 h. Reaction was heated at 60° C for 2 h and then concentrated. Residue was partitioned between EtOAc (4 mL) and water (4 mL) and Boc₂O (10.0 mmol) was added and stirred for 2 h. More water and EtOAc were added and the organics were separated, washed with brine and dried over Na₂SO₄. The crude product was purified by flash chromatography with EtOAc. Yield=260 mg (27%), colourless oil.

LCMS (3 minute method): 1.00 min (239; M+H). ¹H NMR (500 MHz, CDCl₃) δ ppm 8.43 (d, J=6.10 Hz, 2H) 6.81 (d, J=6.10 Hz, 2H) 5.00 (br. s., 1H) 4.08 (t, J=5.11 Hz, 2H) 3.56 (q, J=5.24 Hz, 2H) 1.45 (s, 9 H).

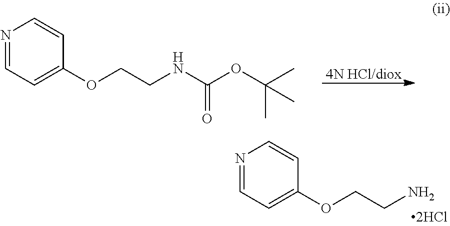

The Boc-protected amine (0.96 mmol) was stirred in 4N HCl in dioxane at RT for 6 h. Concentrated. Yield=200 mg (99%), white solid.

¹H NMR (500 MHz, DMSO-d₆) δ ppm 8.79 (d, J=7.17 Hz, 2H) 8.59 (br. s., 3H) 7.58 (d, J=7.02 Hz, 2H) 4.59 (t, J=4.96 Hz, 2H) 3.28 (d, J=4.58 Hz, 2H).

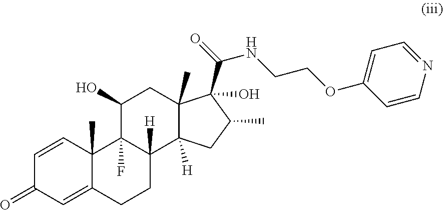

Synthesised by general method A using acid 100a and the amine prepared in step (ii) and purified by flash chromatography with 5-7.5% MeOH:DCM. Yield=35 mg (54%), white solid. LCMS (7 minute method): 2.92 min (499; M+H). HRMS 499.2609 (expected for C28H35FN2O5 499.2608).

¹H NMR (500 MHz, DMSO-d₆) δ ppm 8.39 (d, J=6.10 Hz, 2H) 7.57 (t, J=5.80 Hz, 1H) 7.31 (d, J=10.22 Hz, 1H) 6.99 (d, J=6.26 Hz, 2H) 6.22 (dd, J=10.15, 1.60 Hz, 1H) 6.00 (s, 1H) 5.24 (d, J=3.36 Hz, 1H) 4.75 (s, 1H) 4.05-4.14 (m, 3H) 3.49-3.59 (m, 1H) 3.38-3.46 (m, 1H) 2.97-3.07 (m, 1H) 2.56-2.67 (m, 1H) 2.25-2.41 (m, 2H) 1.99-2.10 (m, 2H) 1.73-1.81 (m, 1H) 1.62 (q, J=11.55 Hz, 1H) 1.49 (s, 3H) 1.28-1.45 (m, 2H) 1.06 (ddd, J=11.90, 8.09, 4.12 Hz, 1H) 0.94 (s, 3H) 0.80 (d, J=7.17 Hz, 3H).

### Example 13

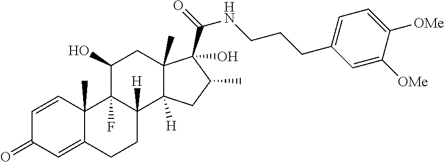

Synthesised by general method A using acid 100a and purified by flash chromatography with 50-75% EtOAc:heptanes and then by mass directed prep HPLC. Yield=41 mg (57%), white solid. LCMS (7 minute method): 4.17 min (556; M+H). HRMS 556.3071 (expected for C₃₂H₄₂FNO₆ 556.3074).

¹H NMR (500 MHz, MeOD) δ ppm 7.57 (t, J=5.49 Hz, 1H) 7.41 (d, J=10.22 Hz, 1H) 6.79-6.90 (m, 2H) 6.76 (d, J=8.24 Hz, 1H) 6.28 (d, J=10.22 Hz, 1H) 6.08 (s, 1H) 4.18-4.29 (m, 1H) 3.82 (s, 3H) 3.79 (s, 3H) 3.07-3.22 (m, 2H) 2.72 (td, J=13.47, 5.72 Hz, 1H) 2.60 (t, J=7.63 Hz, 2H) 2.34-2.53 (m, 2H) 2.13-2.25 (m, 2H) 1.69-1.93 (m, 4 H) 1.41-1.63 (m, 5H) 1.20 (ddd, J=12.13, 8.32, 4.12 Hz, 1H) 1.10 (s, 3H) 0.89 (d, J=7.32 Hz, 3H).

### Example 14

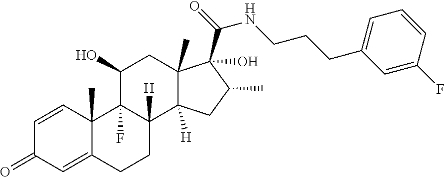

Synthesised by general method A using acid 100a and purified by flash chromatography with 50% EtOAc:heptanes. Yield=66 mg (98%), white solid. LCMS (7 minute method): 4.41 min (514; M+H). HRMS 514.2769 (expected for C₃₀H₃₇F₂NO₄ 514.2769).

¹H NMR (500 MHz, MeOD) δ ppm 7.41 (d, J=10.22 Hz, 1H) 7.24-7.30 (m, 1H) 7.03 (d, J=7.63 Hz, 1H) 6.96 (d, J=10.22 Hz, 1H) 6.89 (td, J=8.58, 2.37 Hz, 1H) 6.28 (dd, J=10.07, 1.53 Hz, 1H) 6.08 (s, 1H) 4.23 (dd, J=11.06, 1.60 Hz, 1H) 3.26-3.35 (m, 1H) 3.07-3.22 (m, 2H) 2.63-2.77 (m, 3H) 2.35-2.52 (m, 2H) 2.14-2.26 (m, 2H) 1.70-1.93 (m, 4 H) 1.59 (s, 3H) 1.43-1.57 (m, 2H) 1.17-1.22 (m, 1H) 1.10 (s, 3H) 0.90 (d, J=7.17 Hz, 3H).

### Example 15

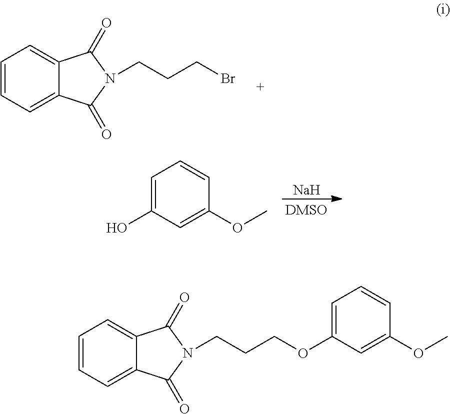

Procedure as used to make the amine in Example 9. Yield=1.069 g (69%), white solid. LCMS (3 minute method): 2.23 min (312; M+H). ¹H NMR (500 MHz, CDCl₃) δ ppm 7.85 (dd, J=5.34, 3.05 Hz, 2H) 7.72 (dd, J=5.42, 3.13 Hz, 2H) 7.14 (t, J=8.24 Hz, 1H) 6.49 (dd, J=8.24, 2.14 Hz, 1H) 6.42 (dd, J=8.09, 1.98 Hz, 1H) 6.37 (t, J=2.21 Hz, 1H) 4.02 (t, J=6.03 Hz, 2H) 3.91 (t, J=6.94 Hz, 2H) 3.75 (s, 3H) 2.19 (quin, J=6.45 Hz, 2H).

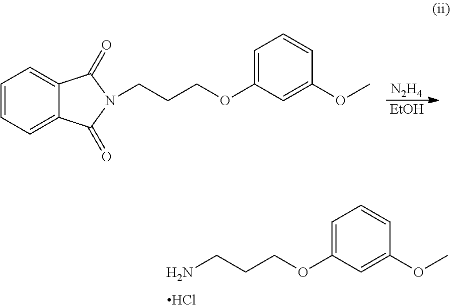

Procedure as used to make the amine in Example 9. Yield=705 mg (96%), white solid. ¹H NMR (500 MHz, DMSO-d₆) δ ppm 8.19 (br. s., 3H) 7.17 (t, J=8.09 Hz, 1H) 6.33-6.66 (m, 3H) 4.04 (t, J=6.26 Hz, 2H) 3.72 (s, 3H) 2.92 (br. s., 2H) 1.95-2.09 (m, 2H).

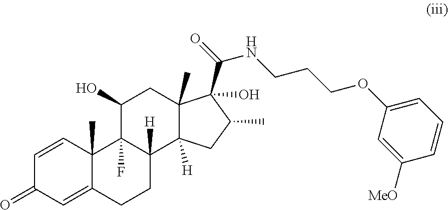

Synthesised by general method A using acid 100a and the amine prepared in step (ii) and purified by flash chromatography with 30-50% EtOAc:heptanes. Yield=49 mg (70%), white solid. LCMS (7 minute method): 4.35 min (542; M+H). HRMS 542.2927 (expected for C₃₁H₄₀FNO₆ 542.2918).

¹H NMR (500 MHz, MeOD) δ ppm 7.41 (d, J=10.07 Hz, 1H) 7.09-7.20 (m, 1H) 6.45-6.56 (m, 3H) 6.28 (dd, J=10.07, 1.83 Hz, 1H) 6.08 (s, 1H) 4.20 (dd, J=10.91, 1.91 Hz, 1H) 4.04 (t, J=6.03 Hz, 2H) 3.75 (s, 3H) 3.42-3.52 (m, 1H) 3.36 (dt, J=13.47, 6.62 Hz, 1H) 3.08-3.18 (m, 1H) 2.67-2.77 (m, 1H) 2.35-2.52 (m, 2H) 2.15-2.27 (m, 2H) 1.95-2.02 (m, 2H) 1.84-1.92 (m, 1H) 1.70-1.81 (m, 1H) 1.59 (s, 3H) 1.43-1.57 (m, 2H) 1.16-1.22 (m, 1H) 1.09 (s, 3H) 0.89 (d, J=7.32 Hz, 3H).

### Example 16

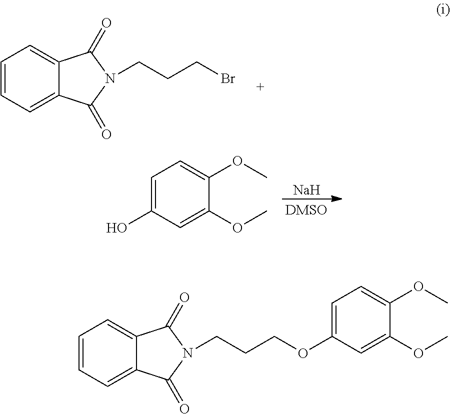

Procedure as used to make the amine in Example 9. Purified by flash chromatography with 0.5 MeOH:DCM. Yield=1.178 g (69%), white solid. LCMS (3 minute method): 2.08 min (342; M+H). ¹H NMR (500 MHz, CDCl₃)δ ppm 7.85 (dd, J=5.49, 3.05 Hz, 2H) 7.72 (dd, J=5.34, 3.05 Hz, 2H) 6.74 (d, J=8.70 Hz, 1H) 6.39 (d, J=2.59 Hz, 1H) 6.34 (dd, J=8.70, 2.59 Hz, 1H) 3.99 (t, J=5.95 Hz, 2H) 3.92 (t, J=6.87 Hz, 2H) 3.82 (s, 3H) 3.80 (s, 3H) 2.17 (quin, J=6.41 Hz, 2H).

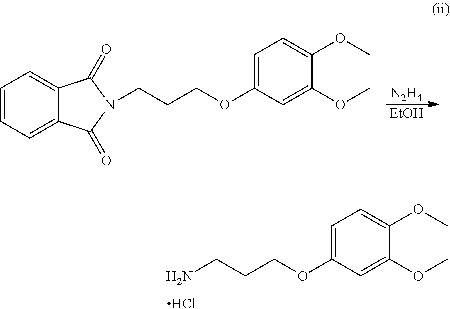

Procedure as used to make the amine in Example 9. Yield=802 mg (95%), pinkish solid. ¹H NMR (500 MHz, DMSO-d₆) δ ppm 8.22 (br. s., 3H) 6.84 (d, J=8.85 Hz, 1H) 6.59 (d, J=2.75 Hz, 1H) 6.42 (dd, J=8.77, 2.82 Hz, 1H) 4.00 (t, J=6.18 Hz, 2H) 3.73 (s, 3H) 3.67 (s, 3H) 2.85-2.98 (m, 2H) 2.02 (quin, J=6.79 Hz, 2H).

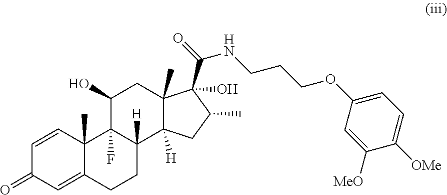

Synthesised by general method A using acid 100a and purified by flash chromatography with 50-70% EtOAc:heptanes. Yield=43 mg (58%), white solid. LCMS (7 minute method): 4.09 min (572; M+H). HRMS 572.3008 (expected for C32H42FNO7 572.3024).

¹H NMR (500 MHz, MeOD) δ ppm 7.40 (d, J=10.22 Hz, 1H) 6.84 (d, J=8.85 Hz, 1H) 6.62 (d, J=2.75 Hz, 1H) 6.46 (dd, J=8.70, 2.75 Hz, 1H) 6.28 (dd, J=10.07, 1.83 Hz, 1H) 6.08 (s, 1H) 4.19 (ddd, J=10.95, 3.62, 1.75 Hz, 1H) 4.02 (t, J=5.87 Hz, 2H) 3.79 (s, 3H) 3.76 (s, 3H) 3.43-3.52 (m, 1H) 3.37 (dt, J=13.35, 6.60 Hz, 1H) 3.08-3.19 (m, 1H) 2.66-2.78 (m, 1H) 2.35-2.53 (m, 2H) 2.15-2.28 (m, 2H) 1.95-2.01 (m, 2H) 1.84-1.93 (m, 1H) 1.71-1.81 (m, 1H) 1.58 (s, 3H) 1.43-1.57 (m, 2H) 1.20 (ddd, J=12.44, 8.39, 4.50 Hz, 1H) 1.09 (s, 3H) 0.89 (d, J=7.32 Hz, 3H).

### Example 17

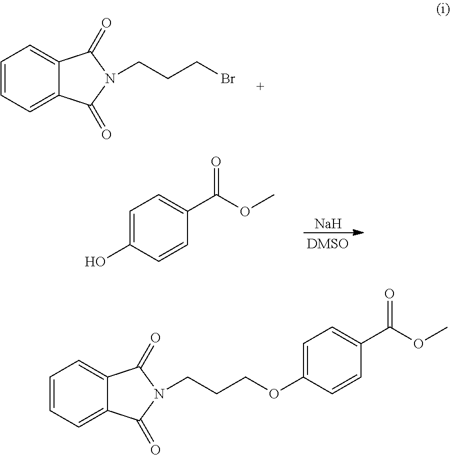

Procedure as used to make the amine in Example 9. Yield=1.659 g (98%), white solid. LCMS (3 minute method): 2.18 min (340; M+H). ¹H NMR (500 MHz, CDCl₃) δ ppm 7.92-7.97 (m, 2H) 7.82-7.87 (m, 2H) 7.70-7.76 (m, 2H) 6.78-6.83 (m, 2H) 4.09 (t, J=5.95 Hz, 2H) 3.93 (t, J=6.79 Hz, 2H) 3.88 (s, 3H) 2.22 (quin, J=6.41 Hz, 2H).

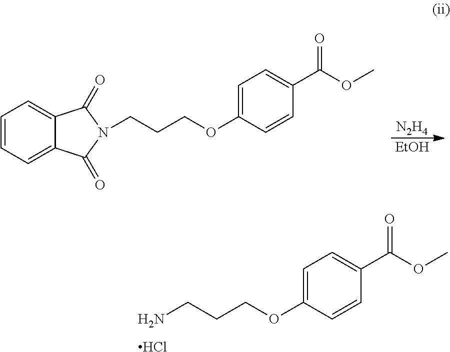 [SMILES: "EtOH" ; COC(=O)c1ccc(OCCCN2C(=O)c3ccccc3C2=O)cc1 ; "N2H4" ; COC(=O)c1ccc(OCCCN)cc1 ; Cl]

Procedure as used to make the amine in Example 9. Yield=576 mg (96%), white solid. ¹H NMR (500 MHz, DMSO-d₆) δ ppm 8.16 (br. s., 3H) 7.91 (d, J=8.85 Hz, 2H) 7.05 (d, J=8.85 Hz, 2H) 4.15 (t, J=6.10 Hz, 2H) 3.81 (s, 3H) 2.87-3.02 (m, 2H) 2.06 (quin, J=6.75 Hz, 2H).

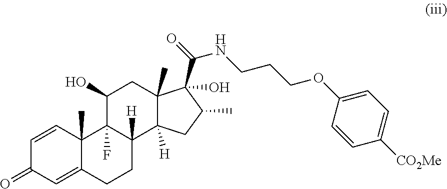

Synthesised by general method A using acid 100a and purified by flash chromatography with 60% EtOAc:heptanes. Yield=144 mg (97%), white solid. LCMS (7 minute method): 4.28 min (570; M+H). HRMS 570.2861 (expected for C₃₂H₄₀FNO₇ 570.2867)

¹H NMR (500 MHz, MeOD) δ ppm 7.91-8.01 (m, 2H) 7.40 (d, J=10.22 Hz, 1H) 6.96-7.06 (m, 2H) 6.28 (dd, J=10.15, 1.75 Hz, 1H) 6.08 (s, 1H) 4.19 (dd, J=10.99, 1.98 Hz, 1H) 4.13 (t, J=6.10 Hz, 2H) 3.87 (s, 3H) 3.42-3.51 (m, 1H) 3.38 (dt, J=13.47, 6.77 Hz, 1H) 3.12 (ddd, J=11.14, 7.17, 4.27 Hz, 1H) 2.66-2.77 (m, 1H) 2.35-2.52 (m, 2H) 2.14-2.25 (m, 2H) 1.99-2.08 (m, 2H) 1.84-1.93 (m, 1H) 1.75 (q, J=12.31 Hz, 1H) 1.59 (s, 3H) 1.52 (qd, J=12.92, 5.19 Hz, 1H) 1.44 (dd, J=13.89, 1.53 Hz, 1H) 1.16-1.23 (m, 1H) 1.08 (s, 3H) 0.89 (d, J=7.32 Hz, 3H).

### Example 18

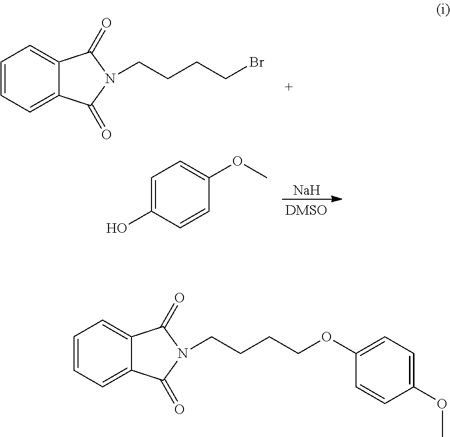

Procedure as used to make the amine in Example 9. Yield=1.14 g (70%), yellow oil. LCMS (3 minute method): 2.21 min (326; M+H). ¹H NMR (500 MHz, CDCl₃) δ ppm 1.75-1.96 (m, 4H) 3.72-3.82 (m, 5H) 3.95 (t, J=6.15 Hz, 2H) 6.82 (s, 4H) 7.72 (dd, J=5.44, 3.07 Hz, 2H) 7.85 (dd, J=5.36, 3.00 Hz, 2H).

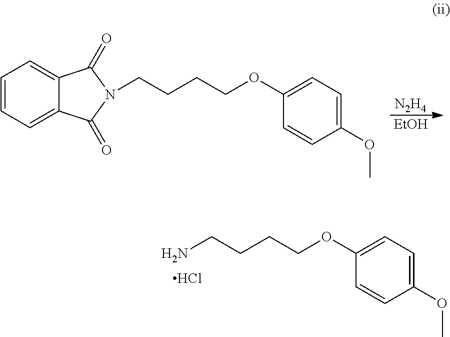

Procedure as used to make the amine in Example 9. Yield=560 mg (69%), pale yellow solid. ¹H NMR (500 MHz, DMSO-d₆) δ ppm 1.59-1.80 (m, 4H) 2.72-2.94 (m, 2H) 3.68 (s, 3H) 3.90 (t, J=5.83 Hz, 2H) 6.85 (s, 4 H) 7.97 (br. s., 2H).

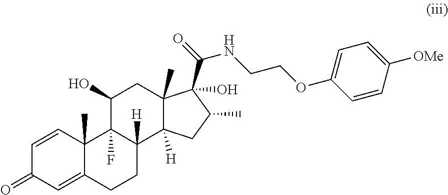

Synthesised by general method A using acid 100a and purified by flash chromatography with 40-60% EtOAc:heptanes. Yield=43 mg (%), white solid. LCMS (7 minute method): 4.30 min (556; M+H). HRMS 556.3076 (expected for C₃₂H₄₂FNO₆ 556.3074).

¹H NMR (500 MHz, MeOD) δ ppm 0.91 (d, J=7.25 Hz, 3H) 1.11 (s, 3H) 1.18-1.25 (m, 1H) 1.42-1.58 (m, 2H) 1.61 (s, 3H) 1.66-1.86 (m, 5H) 1.86-1.98 (m, 1H) 2.13-2.28 (m, 2H) 2.34-2.54 (m, 2H) 2.66-2.80 (m, 1H) 3.09-3.28 (m, 2H) 3.34-3.41 (m, 1H) 3.75 (s, 3H) 3.97 (t, J=6.15 Hz, 2H) 4.20-4.28 (m, 1H) 6.10 (s, 1H) 6.30 (dd, J=10.09, 1.89 Hz, 1H) 6.74-6.94 (m, 4H) 7.34-7.50 (m, 1H) 7.43 (d, J=10.09 Hz, 1H) 7.68 (t, 1H) 7.62-7.75 (m, 1H).

### Example 19

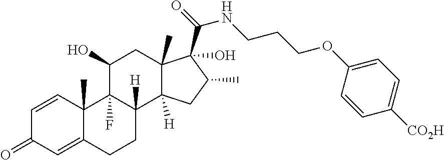

The ester from Example 17 (40 mg, 0.07 mmol) and LiOH.H₂O (9 mg, 0.21 mmol) were dissolved in 1 ml of MeOH/H₂O (1:1). The reaction was stirred at room temperature for 16 h. 1 equivalent of LiOH.H₂O (3 mg, 0.07 mmol) was added and the solution was stirred at room temperature for a further 24 h. Finally, 1 equivalent of LiOH.H₂O (3 mg, 0.07 mmol) was added and the solution was stirred at room temperature until completion by LCMS (72 h). The MeOH was removed under vacuo and the aqueous was acidified to pH˜4 with HCl (1M). the precipitate was filtered, washed with water and dried under vacuo. Yield=21 mg (54%), white solid. LCMS (3 minute method): 1.84 min (556; M+H). HRMS 556.2698 (expected for C₃₁H₃₈FNO₇ 556.2711).

¹H NMR (500 MHz, DMSO-d6) δ ppm 0.80 (d, J=7.25 Hz, 3H) 0.94 (s, 3H) 1.06 (br. s., 1H) 1.32-1.44 (m, 2H) 1.49 (s, 3H) 1.61 (q, J=12.03 Hz, 1H) 1.77 (d, J=6.15 Hz, 1H) 1.87-1.94 (m, 2H) 2.03-2.09 (m, 2H) 2.29-2.38 (m, 2H) 2.58-2.66 (m, 2H) 3.02 (br. s., 1H) 3.17-3.32 (m, 2H) 4.03-4.14 (m, 3H) 4.72 (s, 1H) 5.26 (br. s., 1H) 6.01 (s, 1H) 6.23 (dd, J=10.09, 1.58 Hz, 1H) 7.00 (d, J=8.51 Hz, 2H) 7.30 (d, J=10.09 Hz, 1H) 7.56 (t, J=5.83 Hz, 1H) 7.89 (d, J=8.67 Hz, 2H).

### Example 20

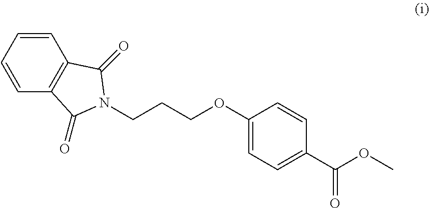 [SMILES: COC(=O)c1ccc(OCCCN2C(=O)c3ccccc3C2=O)cc1]

Sodium hydride (60% suspension in oil; 6.4 g; 0.16 mol) was added to DMSO (100 mL) and stirred until gas evolution ceased. A solution of methyl 4-hydroxybenzoate (24.3 g; 0.16 mol) in DMSO (50 mL) was added dropwise over 1 h with cooling then stirred for a further hour at RT. A solution of N-(3-bromopropyl)phthalimide (42.9 g; 0.16 mol) in DMSO (150 mL) was added over 5 min and the reaction stirred at RT for 3.5 h before pouring onto water (2.1 L), stirring for 10 min, filtering and washing with water. The solid was dissolved in DCM (300 mL), separated and dried (MgSO₄). The product was obtained after filtration and evaporation. Yield=54.3g (100%). LCMS (3 min method): 2.17 min-308 (M−OMe), 326 (M−Me+H), 362 (M+Na). ¹H NMR (500 MHz, DMSO-d6) δ ppm 2.07 (quin, J=6.27 Hz, 2H) 3.76 (t, J=6.70 Hz, 2H) 3.79 (s, 3H) 4.09 (t, J=5.91 Hz, 2H) 6.88 (d, J=8.83 Hz, 2H) 7.80-7.88 (m, 6 H).

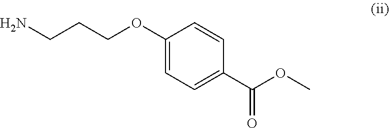

Methyl 4-[3-(1,3-dioxo-2,3-dihydro-1H-isoindol-2-yl)propoxy]benzoate (60 g; 0.18mol) was dissolved in ethanol (1 L) with hydrazine hydrate (11 mL; 0.35 mol) and heated at reflux. After 4 h, the solvent was removed by rotary thin film evaporation and the residue partitioned between EtOAc (400 mL) and 1N NaOH (400 mL) with undissolved solid being removed by filtration. The layers were separated and the organic phase was washed with more 1N NaOH (50 mL) before drying (Na₂SO₄), filtering and evaporating to leave the desired compound. Yield=33.8 g (91%). LCMS (3 min method): 1.13 min-210 (M+H). 1H NMR (500 MHz, DMSO-d6) δ_{H} ppm 1.80 (quin, J=6.54 Hz, 2H) 2.69 (t, J=6.70 Hz, 2H) 3.80 (s, 3H) 4.10 (t, J=6.46 Hz, 2H) 7.03 (d, J=8.83 Hz, 2H) 7.89 (d, J=8.83 Hz, 2H).

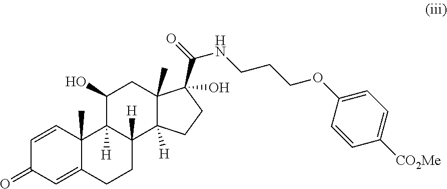

Acid 100c (1.0 g; 2.89 mmol) was dissolved in anhydrous DMF (5 mL) and PyBOP (1.65g; 3.18 mmol) added. The reaction was stirred for 1 h at RT before adding methyl 4-(3-aminopropoxy)benzoate (1.21 g; 5.78 mmol) and heating at 60° C. for 2.5 h. The mixture was cooled and water (20 mL) added. The supernatant was decanted and the residual oil washed with further water (20 mL). The residue was dissolved in EtOAc (20 mL) and washed with 1N HCl (2×20 mL) and saturated sodium bicarbonate (20 mL). The residue after evaporation was purified by chromatography on silica gel with 50-80% EtOAc/heptane then recrystallisation from acetonitrile then methanol. It was very difficult to reduce residual solvent in the crystals below 6%. Yield=840 mg (54%). LCMS (7 min method): 4.04 min-520 (M−OH), 538 (M+H), 560 (M+Na). HRMS—C₃₁H₃₉NO₇—Expected mass: 538.2805, Found: 538.2792, Error=−2.4 ppm. ¹H NMR (500 MHz, DMSO-d6) δ_{H} ppm 0.84 (s, 3H) 0.87 (dd, J=11.03, 3.31 Hz, 1H) 1.00 (qd, J=12.82, 3.78 Hz, 1H) 1.22-1.34 (m, 1H) 1.39 (s, 3H) 1.40-1.47 (m, 1H) 1.50 (dd, J=13.79, 1.97 Hz, 1H) 1.53-1.67 (m, 2H) 1.77 (dd, J=13.79, 3.39 Hz, 1H) 1.84-1.95 (m, 2H) 1.95-2.06 (m, 2H) 2.28 (dd, J=13.00, 3.23 Hz, 1H) 2.52-2.58 (m, 1H) 2.60-2.70 (m, 1H) 3.17-3.23 (m, 1H) 3.29 (dq, J=13.10, 6.61 Hz, 1H) 3.81 (s, 3H) 4.07 (t, J=6.38 Hz, 2H) 4.20-4.27 (m, 1H) 4.62 (d, J=2.99 Hz, 1H) 5.05 (s, 1H) 5.91 (s, 1H) 6.16 (dd, J=10.09, 1.73 Hz, 1H) 7.03 (d, J=8.99 Hz, 2H) 7.32 (d, J=10.09 Hz, 1H) 7.62 (t, J=5.91 Hz, 1H) 7.91 (d, J=8.83 Hz, 2H).

### Example 21

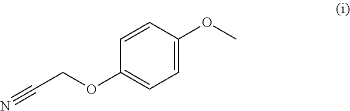

4-Methoxyphenol (5.0 g; 40 mmol), bromoacetonitrile (4.8 mL; 69 mmol) and potassium carbonate (27.8 g; 202 mmol) were dissolved/suspended in THF (100 mL) and heated at 50° C. for 6 h. Much of the solvent was removed by rotary thin film evaporation before adding water (50 mL) and extracting with EtOAc (50 mL). The organic phase was washed with sodium bicarbonate solution (75 mL) and brine before evaporating to dryness and heating the residual oil with heptane (30 mL). After cooling, the supernatant was decanted and the residue chromatographed on silica gel with DCM/heptane (50-100%) to give the title compound. Yield=3.74 g (57%). 1H NMR (500 MHz, CDCl₃) δ_{H} ppm 3.80 (s, 3H) 4.72 (s, 2H) 6.86-6.91 (m, 2H) 6.94-6.99 (m, 2H).

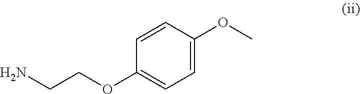

2-(4-Methoxyphenoxy)acetonitrile (3.74 g; 22.9 mmol) was dissolved in diethyl ether (37 mL) and cooled on ice. A solution of lithium aluminium hydride (1M in THF; 68.8 mL; 68.8 mmol) was added dropwise over 45 min before the ice-bath was removed and the reaction stirred at RT for 1 h. Water (2.6 mL), 15% NaOH (2.6 mL) and more water (7.8 mL) were then added cautiously in that order. The solid was filtered off and washed with THF. The solvent was removed by rotary thin film evaporation and the residue dissolved in EtOAc (30 mL). This was extracted with 1N HCl (2×20 mL) and the combined acid layers basified with 4N NaOH (15 mL). The desired compound separated as an oil which was extracted with EtOAc (2×25 mL), dried (Na₂SO₄), filtered and the solvent removed by rotary thin film evaporation. Yield=2.21 g (58%). LCMS (3 min method): 0.80 min-168 (M+H). ¹H NMR (500 MHz, CDCl₃) δ_{H} ppm 3.06 (t, J=5.19 Hz, 2H) 3.78 (s, 3H) 3.95 (t, J=5.19 Hz, 2H) 6.82-6.88 (m, 4H).

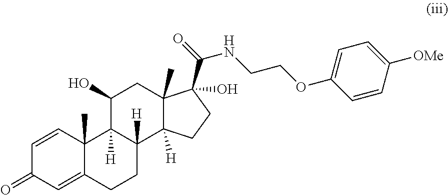

Acid 100c (1.5 g; 4.33 mmol) was dissolved in anhydrous DMF (7.5 mL) and PyBOP (2.48 g; 4.77 mmol) added. The reaction was stirred for 1 h at RT before adding 1-(2-aminoethoxy)-4-methoxybenzene (1.09 g; 6.50 mmol) and triethylamine (0.60 mL; 4.33 mmol) and heating at 60° C. for 1.5 h. The mixture was cooled and 1N HCl (20 mL) and water (20 mL) added. The supernatant was decanted and the residual oil washed with further water (10 mL). The residue was dissolved in EtOAc (50 mL) and washed with 1N HCl (30 mL) and saturated sodium bicarbonate (20 mL). The residue after evaporation was purified by columning twice on silica gel with 50-80% EtOAc/heptane to give the desired compound as a cream foam. Yield=1.61 g (75%). LCMS (7 min method): 3.94 min-478 (M−OH), 496 (M+H). HRMS—C₂₉H₃₇NO₆—Expected mass: 496.2699, Found: 496.27, Error=0.2 ppm.

1H NMR (500 MHz, DMSO-d6) δH ppm 0.85 (s, 3H) 0.86-0.90 (m, 1H) 1.00 (qd, J=13.02, 4.12 Hz, 1H) 1.30 (qd, J=11.27, 6.03 Hz, 1H) 1.39 (s, 3H) 1.41-1.48 (m, 1H) 1.52 (dd, J=13.81, 2.21 Hz, 1H) 1.54-1.67 (m, 2H) 1.76 (dd, J=13.81, 3.43 Hz, 1H) 1.96-2.06 (m, 2H) 2.29 (dd, J=13.20, 3.13 Hz, 1H) 2.52-2.57 (m, 1H) 2.60-2.70 (m, 1H) 3.36-3.41 (m, 1H) 3.41-3.49 (m, 1H) 3.69 (s, 3H) 3.90 (t, J=6.41 Hz, 2H) 4.22-4.29 (m, 1H) 4.60 (d, J=3.20 Hz, 1H) 5.12 (s, 1H) 5.91 (s, 1H) 6.16 (dd, J=10.07, 1.83 Hz, 1H) 6.83-6.87 (m, 2H) 6.87-6.91 (m, 2H) 7.33 (d, J=10.07 Hz, 1H) 7.56 (t, J=5.95 Hz, 1H).

### Example 22

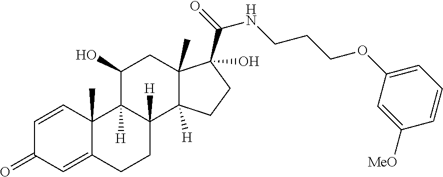

Acid 100c (1.038 g; 3.0 mmol) was dissolved in anhydrous DMF (15 mL) and PyBOP (1.794 g; 3.45 mmol) added. The reaction was stirred for 1 h at RT before adding 1-(3-aminopropoxy)-3-methoxybenzene as prepared in Example 15 (814 mg; 4.5 mmol) and triethylamine (0.85 mL; 5 mmol) and heating at 60° C for 2.5 h. The mixture was cooled and partitioned between 1N HCl (50 mL) and EtOAc (100 mL). The aqueous phases were extracted one more time with EtOAc (100 mL). The combined organic layer was washed with sat. NaHCO₃ (50 mL), dried (Na₂SO₄), filtered and evaporated in vacuo to give a brown oil, which was purified by column (60% EtOAc-40% heptane). The final product was further purified by another column (eluting with 1-4% MeOH in DCM) to give a white foam. Yield=820 mg (53%). LCMS (7 min method): 4.25 min-510 (M⁺+H) and 532 (M⁺+23). HRMS—C₃₀H₃₉NO₆—Expected mass: 510.2856, Found: 510.2869, Error=2.5 ppm.

¹H NMR (500 MHz, CDCl₃) δ_{H} ppm 1.01 (s, 3H) 1.04-1.19 (m, 2H) 1.24-1.33 (m, 1H) 1.40-1.59 (m, 6H) 1.61-1.68 (m, 1H) 1.76-1.86 (m, 1H) 1.95 (dd, J=14.19, 3.78 Hz, 1H) 1.99-2.06 (m, 1H) 2.07-2.16 (m, 1H) 2.33 (dd, J=13.40, 3.31 Hz, 1H) 2.46 (s, 1H) 2.51-2.62 (m, 1H) 2.74-2.85 (m, 1H) 3.38-3.48 (m, 1H) 3.54 (dq, J=13.16, 6.44 Hz, 1H) 3.76-3.83 (m, 2H) 4.00-4.09 (m, 1H) 4.40 (t, J=2.84 Hz, 1H) 6.01 (s, 1H) 6.26 (dd, J=10.09, 1.89 Hz, 1H) 6.43-6.48 (m, 1H) 6.52 (ddd, J=15.76, 8.20, 2.21 Hz, 1H) 6.94 (t, J=5.52 Hz, 1H) 7.20 (t, J=8.35 Hz, 1H) 7.26 (d, J=10.09 Hz, 1H).

### Examples 23-47

### Examples 23-47 were prepared according to the following general scheme.

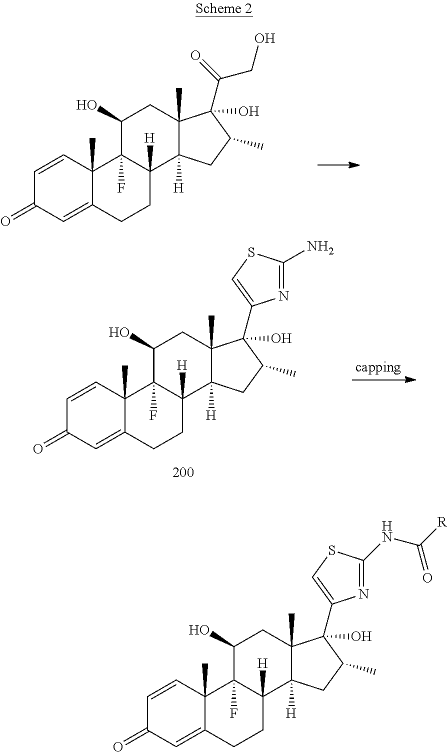

Synthesis of Aminothiazole 200:

(i) Methanesulfonic acid 2-(9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxo-ethyl ester

Dexamethasone (3 g; 7.65 mmol) was dissolved in pyridine (40 ml) and cooled to 0° C. before adding methanesulfonyl chloride (0.88 ml; 11.5 mmol) and stirring at RT for 3 h. The reaction was poured onto ice-water (250 ml) and stirred for 1 h before filtering and washing with water. The dried solid was suspended in dichloromethane (40 ml) and stirred for 10 mins before filtering and drying. Yield=3.45 g (96%). HRMS—C₂₃H₃₁FO₇S—Expected mass: 471.1853, Found: 471.1837, Error=−3.4 ppm. ¹H NMR (500 MHz, MeOD) δ ppm 0.87 (d, J=7.32 Hz, 3H) 1.03 (s, 3H) 1.21 (ddd, J=12.25, 8.20, 4.12 Hz, 1H) 1.45-1.58 (m, 2H) 1.59 (s, 3H) 1.71-1.81 (m, 1H) 1.84-1.93 (m, 1H) 2.22 (td, J=11.83, 8.39 Hz, 1H) 2.32 (dt, J=13.58, 2.90 Hz, 1H) 2.36-2.54 (m, 2H) 2.72 (td, J=13.54, 5.72 Hz, 1H) 3.00-3.12 (m, 1H) 3.19 (s, 3H) 4.23-4.30 (m, 1H) 5.02 (d, J=18.01 Hz, 1H) 5.26 (d, J=18.01 Hz, 1H) 6.08 (s, 1H) 6.29 (dd, J=10.22, 1.83 Hz, 1H) 7.40 (d, J=10.07 Hz, 1H)

(ii) 17-(2-Amino-thiazol-4-yl)-9-fluoro-11,17-dihydroxy-10,13,16-trimethyl-6,7,8,9,10,11,12,13,14,15,16,17-dodecahydro-cyclopenta[a]phenanthren-3-one (200) The mesylate prepared as above (50 mg; 0.11 mmol) was mixed with thiourea (9 mg; 0.12 mmol) in acetonitrile (0.5 ml) in a microwave tube and heated at 100° C for 1 h. Water (2 ml) was added and the precipitate filtered off. The solution was basified with saturated sodium carbonate (pH 10-11) and this precipitate filtered, washed with water and dried. Yield=35 mg (74%). LCMS (7 minute method): 2.87 min (433; M+H). HRMS—C₂₃H₂₉FN₂O₃S—Expected mass: 433.1961, Found: 433.1975, Error=3.2 ppm. ¹H NMR (500 MHz, MeOD) δ ppm 0.93 (s, 3H) 0.98 (d, J=7.17 Hz, 3H) 1.28 (ddd, J=12.21, 8.32, 4.04 Hz, 1H) 1.48-1.61 (m, 4 H) 1.64 (d, J=12.97 Hz, 1H) 1.77 (q, J=11.55 Hz, 1H) 1.85-1.96 (m, 1H) 2.18-2.29 (m, 2H) 2.35-2.49 (m, 2H) 2.72 (td, J=13.54, 5.87 Hz, 1H) 2.86-2.99 (m, 1H) 4.23 (dd, J=11.22, 1.75 Hz, 1H) 6.08 (s, 1H) 6.28 (dd, J=10.15, 1.75 Hz, 1H) 6.33 (s, 1H) 7.43 (d, J=10.22 Hz, 1H)

General Method A:

To aminothiazole 200 (50 mg, 0.12 mmol) in DCM:pyridine (1:1, 2 mL) was added the appropriate acid chloride (0.23 mmol) and stirred at RT for 16 h. When complete, the mixture was loaded onto a 2g basic column and run through with acetonitrile (3 ml). The solvents were evaporated to dryness, azeotroped with heptanes (×3) and purified by chromatography on silica gel if required.

General Method B:

To the appropriate acid (0.14 mmol) in DMF (0.5 mL) was added PyBOP (90 mg, 0.17 mmol) and DIPEA (60 μL, 0.35 mmol) and stirred at RT for 1 h. Aminothiazole 200 (50 mg, 0.12 mmol) was added and reaction heated to 50° C for 2 h. When complete, the mixture was cooled to RT then loaded onto a 2 g basic column and run through with acetonitrile (3 ml). This solution was then loaded onto a 2 g acid column and run with further acetonitrile. The solvents were evaporated to dryness and purified by chromatography on silica gel if required.

General Method C:

To the appropriate acid (0.23 mmol) in DCM (1 mL) was added oxalyl chloride (20 μL, 0.23 mmol) and DMF (1 drop) and stirred at RT for 2 h. This solution was added to aminothiazole 200 in pyridine (1 mL) and stirred at RT for 2 h. When complete, the mixture was loaded onto a 2 g basic column and run through with acetonitrile (3 ml). The solvents were evaporated to dryness, azeotroped with heptanes (×3) and purified by chromatography on silica gel if required.

### Example 23

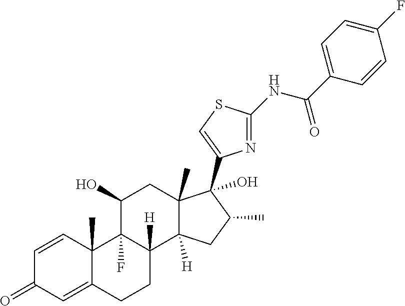

Aminothiazole 200 (40 mg; 0.093 mmol), 4-fluorobenzoyl chloride (16 μl; 0.14 mmol) and triethylamine (19 μl; 0.14 mmol) were suspended in dichloromethane (0.8 ml) and heated at 50° C for 5 h before adding further acid chloride (32 μl) and heating for another 3 h. As the reaction was still incomplete, further triethylamine (38 μl) was added and heating continued for another 4 h. The solvent was then evaporated and the residue dissolved in methanol (2 ml) and 0.88 ammonia (0.2 ml). After 1 h, the solvents were evaporated and the residue partly purified by chromatography with 10-40% EtOAc in heptane. Remains of 4-fluorobenzamide were removed by recrystallisation from 1:1 methanol:water Yield=17 mg (33%). LCMS (7 minute method): 4.70 min (555; M+H). HRMS—C₃₀H₃₂F₂N₂O₄S—Expected mass: 555.2129, Found: 555.2132, Error=0.5 ppm.

¹H NMR (500 MHz, MeOD) δ ppm 0.92 (s, 3H) 1.01 (d, J=7.17 Hz, 3H) 1.33 (ddd, J=12.32, 8.05, 4.04 Hz, 1H) 1.52-1.63 (m, 5H) 1.77-1.89 (m, 1H) 1.90-1.98 (m, 1H) 2.26-2.36 (m, 2H) 2.38-2.53 (m, 2H) 2.68-2.79 (m, 1H) 3.09-3.16 (m, 1H) 4.26 (dt, J=11.18, 2.04 Hz, 1H) 4.59 (s, 1H) 6.09 (s, 1H) 6.29 (dd, J=10.07, 1.83 Hz, 1H) 6.95 (s, 1H) 7.29 (t, J=8.70 Hz, 2H) 7.43 (d, J=10.07 Hz, 1H) 8.09 (dd, J=8.70, 5.34 Hz, 2H).

### Example 24

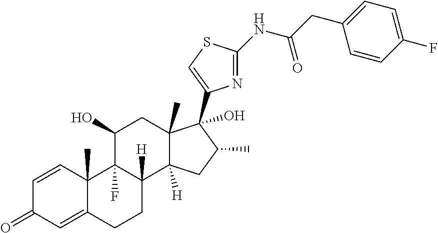

Prepared using method A but on 4 times the scale. Purified by flash chromatography with 30-50% EtOAc:heptanes. Yield=53 mg (20%), white solid. LCMS (3 minute method): 2.30 min (569; M+H).

¹H NMR (500 MHz, MeOD) δ ppm 7.42 (d, J=10.07 Hz, 1H) 7.32-7.38 (m, 2H) 7.03-7.10 (m, 2H) 6.88 (s, 1H) 6.28 (dd, J=10.07, 1.98 Hz, 1H) 6.09 (s, 1H) 4.24 (ddd, J=11.14, 3.74, 1.91 Hz, 1H) 3.75 (s, 2H) 3.11 (ddd, J=10.99, 7.02, 4.12 Hz, 1H) 2.67-2.78 (m, 1H) 2.36-2.50 (m, 2H) 2.23-2.32 (m, 2H) 1.88-1.96 (m, 1H) 1.75-1.85 (m, 1H) 1.51-1.62 (m, 5 H) 1.31 (ddd, J=12.25, 8.28, 4.04 Hz, 1H) 0.98 (d, J=7.17 Hz, 3H) 0.88 (s, 3H).

### Example 25

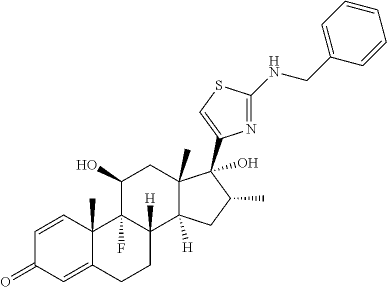

Mesylated dexamethasone (200 mg, 0.43 mmol), N-benzylthiourea (85 mg, 0.51 mmol) and dimethylaniline (215 μL, 1.70 mmol) in MeCN (4 mL) were heated in a sealed tube at 100° C. for 4 h. Reaction cooled and partitioned between EtOAc (20 mL) and water (20 mL). Organics were washed with 1M KHSO4 (20 mL), sat. NaHCOs (20 mL) and dried over Na₂SO₄. Concentrated and purified by flash chromatography with 30% EtOAc:heptanes. Yield=97 mg (42%), white solid. LCMS (7 minute method): 3.96 min (523; M+H). HRMS 523.2417 (expected for C₃₀H₃₅FN₂O₃S 523.2431).

¹H NMR (500 MHz, DMSO-d₆) δ ppm 7.96 (t, J=5.87 Hz, 1H) 7.27-7.40 (m, 5 H) 7.21-7.27 (m, 1H) 6.33 (s, 1H) 6.21 (dd, J=10.07, 1.83 Hz, 1H) 6.00 (s, 1H) 5.09 (d, J=2.14 Hz, 1H) 4.32-4.44 (m, 2H) 4.07-4.15 (m, 1H) 3.95 (s, 1H) 2.86-2.97 (m, 1H) 2.56-2.68 (m, 1H) 2.23-2.38 (m, 2H) 2.03-2.16 (m, 2H) 1.75-1.84 (m, 1H) 1.65 (q, J=11.75 Hz, 1H) 1.44-1.55 (m, 4H) 1.36 (qd, J=12.79, 4.96 Hz, 1H) 1.13 (ddd, J=12.09, 8.20, 3.97 Hz, 1H) 0.86 (d, J=7.02 Hz, 3H) 0.82 (s, 3H).

### Example 26

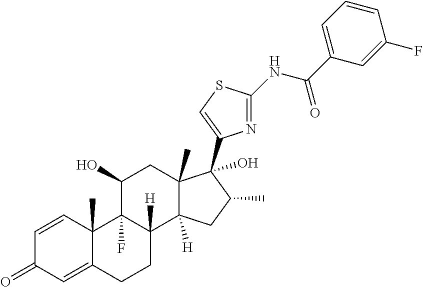

Prepared using method A. Purified by flash chromatography with 25-50% EtOAc:heptanes. Yield=47 mg (73%), white solid. LCMS (7 minute method): 4.81 min (555; M+H). HRMS 555.2148 (expected for C₃₀H₃₂F₂N₂O₄S 555.2129).

¹H NMR (500 MHz, MeOD) δ ppm 7.86 (d, J=7.78 Hz, 1H) 7.76 (dt, J=9.54, 1.95 Hz, 1H) 7.58 (td, J=8.01, 5.65 Hz, 1H) 7.43 (d, J=10.22 Hz, 1H) 7.39 (td, J=8.39, 2.59 Hz, 1H) 6.97 (s, 1H) 6.29 (dd, J=10.15, 1.91 Hz, 1H) 6.09 (s, 1H) 4.23-4.30 (m, 1H) 3.09-3.18 (m, 1H) 2.69-2.78 (m, 1H) 2.38-2.52 (m, 2H) 2.26-2.35 (m, 2H) 1.90-1.98 (m, 1H) 1.78-1.88 (m, 1H) 1.52-1.68 (m, 5 H) 1.25-1.37 (m, 1H) 1.01 (d, J=7.32 Hz, 3H) 0.92 (s, 3H).

### Example 27

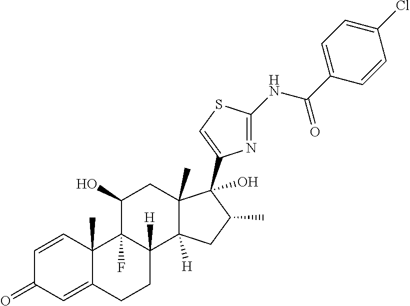

Prepared using method A. Purified by flash chromatography with 30-50% EtOAc:heptanes. Yield=64 mg (97%), white solid. LCMS (7 minute method): 4.95 min (571; M(³⁵Cl)+H). HRMS 571.1819 (expected for C₃₀H₃₂ClFN₂O₄S 571.1834).

¹H NMR (500 MHz, MeOD) δ ppm 8.01 (d, J=8.54 Hz, 2H) 7.57 (d, J=8.54 Hz, 2H) 7.43 (d, J=10.22 Hz, 1H) 6.96 (s, 1H) 6.29 (dd, J=10.07, 1.83 Hz, 1H) 6.09 (s, 1H) 4.26 (dd, J=11.06, 1.91 Hz, 1H) 3.09-3.19 (m, 1H) 2.68-2.79 (m, 1H) 2.37-2.52 (m, 2H) 2.26-2.35 (m, 2H) 1.90-1.97 (m, 1H) 1.77-1.87 (m, 1H) 1.52-1.66 (m, 5 H) 1.28-1.37 (m, 1H) 1.01 (d, J=7.17 Hz, 3H) 0.92 (s, 3H).

### Example 28

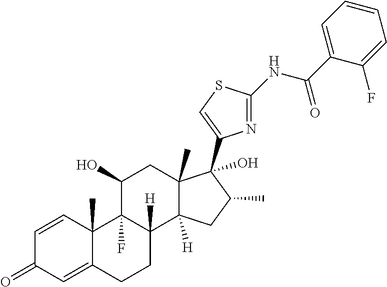

Prepared using method A. Purified by flash chromatography with 25-50% EtOAc:heptanes. Yield=57 mg (89%), white solid. LCMS (7 minute method): 4.73 min (555; M+H). HRMS 555.2145 (expected for C₃₀H₃₂F₂N₂O₄S 555.2129).

¹H NMR (500 MHz, MeOD) δ ppm 7.84 (td, J=7.51, 1.75 Hz, 1H) 7.61-7.66 (m, 1H) 7.43 (d, J=10.07 Hz, 1H) 7.36 (td, J=7.63, 0.92 Hz, 1H) 7.30 (dd, J=10.91, 8.47 Hz, 1H) 6.98 (s, 1H) 6.29 (dd, J=10.15, 1.91 Hz, 1H) 6.09 (s, 1H) 4.26 (ddd, J=11.10, 3.78, 1.91 Hz, 1H) 3.12 (ddd, J=10.99, 7.10, 4.04 Hz, 1H) 2.69-2.78 (m, 1H) 2.37-2.51 (m, 2H) 2.26-2.35 (m, 2H) 1.90-1.97 (m, 1H) 1.78-1.87 (m, 1H) 1.52-1.67 (m, 5 H) 1.26-1.36 (m, 1H) 1.01 (d, J=7.32 Hz, 3H) 0.92 (s, 3H).

### Example 29

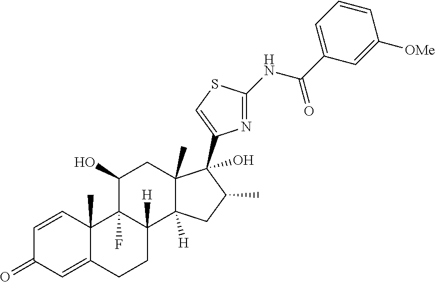

Prepared using method A. Purified by flash chromatography with 30-50% EtOAc:heptanes. Yield=48 mg (73%), white solid. LCMS (7 minute method): 4.77 min (567; M+H). HRMS 567.2324 (expected for C₃₁H₃₅FN₂O₅S 567.2329).

¹H NMR (500 MHz, MeOD) δ ppm 7.55-7.61 (m, 2H) 7.40-7.49 (m, 2H) 7.17-7.22 (m, 1H) 6.95 (s, 1H) 6.29 (dd, J=10.15, 1.91 Hz, 1H) 6.09 (s, 1H) 4.26 (ddd, J=11.02, 3.78, 1.83Hz, 1H) 3.89 (s, 3H) 3.08-3.18 (m, 1H) 2.68-2.78 (m, 1H) 2.38-2.52 (m, 2H) 2.26-2.35 (m, 2H) 1.90-1.97 (m, 1H) 1.83 (q, J=11.24 Hz, 1H) 1.51-1.68 (m, 5 H) 1.25-1.37 (m, 1H) 1.02 (d, J=7.17 Hz, 3H) 0.92 (s, 3H).

### Example 30

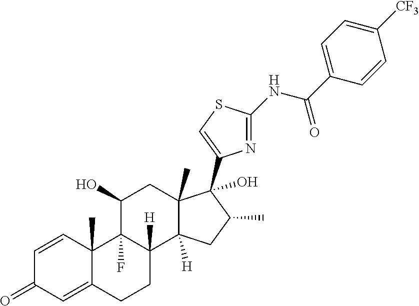

Prepared using method A. Purified by flash chromatography with 40% EtOAc:heptanes. Yield=66 mg (95%), white solid. LCMS (7 minute method): 5.03 min (605; M+H). HRMS 605.2110 (expected for C₃₁H₃₂F₄N₂O₄S 605.2097).

¹H NMR (500 MHz, MeOD) δ ppm 8.19 (d, J=8.09 Hz, 2H) 7.87 (d, J=8.24 Hz, 2H) 7.43 (d, J=10.07 Hz, 1H) 6.98 (s, 1H) 6.29 (dd, J=10.15, 1.91 Hz, 1H) 6.09 (s, 1H) 4.26 (dd, J=11.06, 1.91 Hz, 1H) 3.17 (dt, J=3.24, 1.66 Hz, 1H) 2.68-2.79 (m, 1H) 2.38-2.52 (m, 2H) 2.26-2.36 (m, 2H) 1.90-1.98 (m, 1H) 1.83 (q, J=11.24 Hz, 1H) 1.51-1.67 (m, 5H) 1.26-1.37 (m, 1H) 1.01 (d, J=7.17 Hz, 3H) 0.92 (s, 3H).

### Example 31

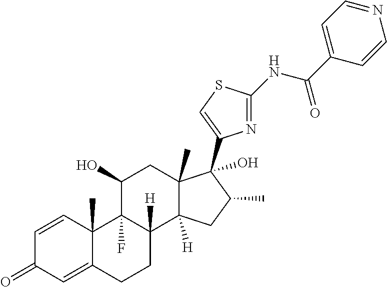

Prepared using method A. Purified by flash chromatography with 50-100% EtOAc:heptanes. Yield=57 mg (92%), white solid. LCMS (7 minute method): 4.13 min (538; M+H). HRMS 538.2195 (expected for C₂₉H₃₂FN₃O₄S 538.2176).

¹H NMR (500 MHz, MeOD) δ ppm 8.77 (d, J=5.95 Hz, 2H) 7.97 (d, J=5.65 Hz, 2H) 7.43 (d, J=10.07 Hz, 1H) 7.00 (s, 1H) 6.29 (dd, J=10.15, 1.91 Hz, 1H) 6.09 (s, 1H) 4.26 (dd, J=11.06, 1.91 Hz, 1H) 3.10-3.21 (m, 1H) 2.68-2.79 (m, 1H) 2.37-2.52 (m, 2H) 2.26-2.36 (m, 2H) 1.90-1.98 (m, 1H) 1.83 (q, J=11.70 Hz, 1H) 1.51-1.65 (m, 5 H) 1.28-1.37 (m, 1H) 1.01 (d, J=7.17 Hz, 3H) 0.92 (s, 3H).

### Example 32

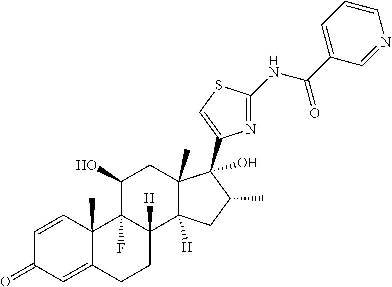

Prepared using method A. Purified by flash chromatography with 50-100% EtOAc:heptanes. Yield=50 mg (81%), white solid. LCMS (7 minute method): 4.16 min (538; M+H). HRMS 538.2194 (expected for C₂₉H₃₂FN₃O₄S 538.2176).

¹H NMR (500 MHz, MeOD) δ ppm 9.17 (br. s., 1H) 8.76 (d, J=3.97 Hz, 1H) 8.43 (d, J=7.93 Hz, 1H) 7.62 (dd, J=7.86, 4.96 Hz, 1H) 7.43 (d, J=10.07 Hz, 1H) 6.98 (s, 1H) 6.29 (dd, J=10.07, 1.83 Hz, 1H) 6.09 (s, 1H) 4.26 (dd, J=11.06, 1.75 Hz, 1H) 3.09-3.20 (m, 1H) 2.68-2.79 (m, 1H) 2.38-2.52 (m, 2H) 2.26-2.36 (m, 2H) 1.89-1.98 (m, 1H) 1.83 (q, J=11.65 Hz, 1H) 1.52-1.66 (m, 5 H) 1.33 (ddd, J=12.25, 8.28, 4.04 Hz, 1H) 1.01 (d, J=7.17 Hz, 3H) 0.93 (s, 3H).

### Example 33

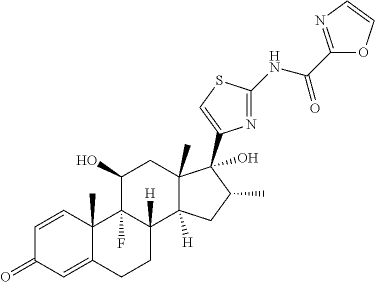

Prepared using method B. Purified by flash chromatography with 50-60% EtOAc:heptanes. Yield=31 mg (51%), white solid. LCMS (7 minute method): 4.23 min (528; M+H). HRMS 528.1949 (expected for C₂₇H₃₀FN₃O₅S 528.1968).

¹H NMR (500 MHz, MeOD) δ ppm 8.18 (s, 1H) 7.46 (s, 1H) 7.43 (d, J=10.07 Hz, 1H) 7.02 (s, 1H) 6.29 (dd, J=10.15, 1.91 Hz, 1H) 6.09 (s, 1H) 4.23-4.30 (m, 1H) 3.06-3.18 (m, 1H) 2.68-2.78 (m, 1H) 2.37-2.52 (m, 2H) 2.27-2.36 (m, 2H) 1.90-1.97 (m, 1H) 1.78-1.88 (m, 1H) 1.51-1.67 (m, 5H) 1.33 (ddd, J=12.25, 8.28, 4.04 Hz, 1H) 1.01 (d, J=7.17 Hz, 3H) 0.91 (s, 3H).

### Example 34

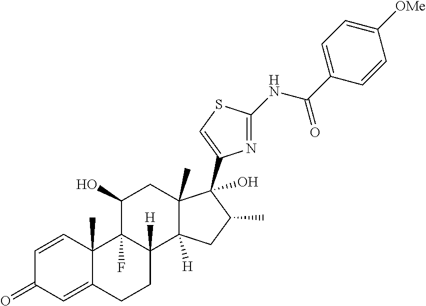

Prepared using method A. Purified by flash chromatography with 30-50% EtOAc:heptanes. Yield=59 mg (90%), white solid. LCMS (7 minute method): 4.71 min (567; M+H). HRMS 567.2325 (expected for C₃₁H₃₅FN₂O₅S 567.2329)

¹H NMR (500 MHz, MeOD) δ ppm 7.97-8.03 (m, 2H) 7.43 (d, J=10.07 Hz, 1H) 7.04-7.10 (m, 2H) 6.92 (s, 1H) 6.29 (dd, J=10.15, 1.91 Hz, 1H) 6.09 (s, 1H) 4.26 (dt, J=11.14, 1.83 Hz, 1H) 3.89 (s, 3H) 3.12 (ddd, J=10.95, 7.06, 4.12 Hz, 1H) 2.68-2.78 (m, 1H) 2.37-2.52 (m, 2H) 2.25-2.36 (m, 2H) 1.90-1.98 (m, 1H) 1.82 (q, J=11.24 Hz, 1H) 1.51-1.68 (m, 5 H) 1.33 (ddd, J=12.21, 8.24, 4.12 Hz, 1H) 1.01 (d, J=7.17 Hz, 3H) 0.92 (s, 3H).

### Example 35

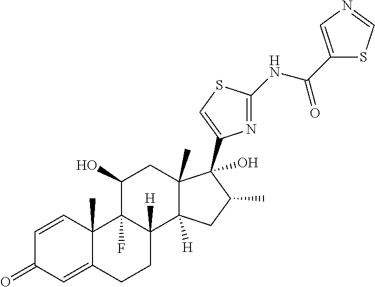

Prepared using method B. Purified by flash chromatography with 50-70% EtOAc:heptanes. Yield=36 mg (57%), white solid. LCMS (7 minute method): 4.30 min (544; M+H). HRMS 544.1754 (expected for C₂₇H₃₀FN₃O₄S₂ 544.1740).

¹H NMR (500 MHz, MeOD) δ ppm 9.22 (br. s., 1H) 8.65 (br. s., 1H) 7.43 (d, J=10.22 Hz, 1H) 6.95 (br. s., 1H) 6.29 (dd, J=10.07, 1.83 Hz, 1H) 6.09 (s, 1H) 4.26 (dd, J=11.06, 1.75 Hz, 1H) 3.06-3.20 (m, 1H) 2.68-2.79 (m, 1H) 2.38-2.52 (m, 2H) 2.25-2.37 (m, 2H) 1.89-1.98 (m, 1H) 1.83 (q, J=11.65 Hz, 1H) 1.51-1.65 (m, 5 H) 1.33 (ddd, J=12.28, 8.24, 4.20 Hz, 1H) 1.01 (d, J=7.17 Hz, 3H) 0.93 (s, 3H).

### Example 36

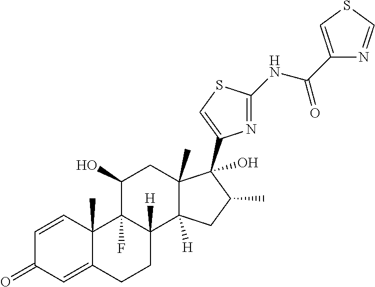

Prepared using method B. Purified by flash chromatography with 50-100% EtOAc:heptanes. Yield=53 mg (84%), white solid. LCMS (7 minute method): 4.42 min (544; M+H). HRMS 544.1752 (expected for C₂₇H₃₀FN₃O₄S₂ 544.1740).

¹H NMR (500 MHz, MeOD) δ ppm 9.12 (d, J=1.98 Hz, 1H) 8.52 (d, J=1.98 Hz, 1H) 7.43 (d, J=10.07 Hz, 1H) 6.99 (s, 1H) 6.29 (dd, J=10.07, 1.83 Hz, 1H) 6.09 (s, 1H) 4.27 (ddd, J=11.18, 3.78, 1.83 Hz, 1H) 3.11 (ddd, J=10.99, 7.10, 4.04 Hz, 1H) 2.67-2.79 (m, 1H) 2.38-2.52 (m, 2H) 2.26-2.36 (m, 2H) 1.90-1.98 (m, 1H) 1.78-1.89 (m, 1H) 1.51-1.70 (m, 5 H) 1.30-1.37 (m, 1H) 1.02 (d, J=7.17 Hz, 3H) 0.91 (s, 3H).

### Example 37

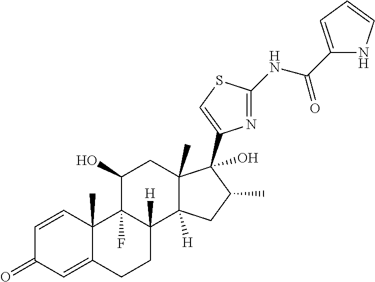

Prepared using method C. Purified by flash chromatography with 30-50% EtOAc:heptanes. Yield=32 mg (52%), white solid. LCMS (7 minute method): 4.34 min (526; M+H). HRMS 526.2181 (expected for C₂₈H₃₂FN₃O₄S 526.2176).

¹H NMR (500 MHz, MeOD) δ ppm 7.43 (d, J=10.07 Hz, 1H) 7.09 (dd, J=3.81, 1.22 Hz, 1H) 7.06 (dd, J=2.44, 1.37 Hz, 1H) 6.86 (s, 1H) 6.25-6.32 (m, 2H) 6.09 (s, 1H) 4.23-4.29 (m, 1H) 3.04-3.16 (m, 1H) 2.68-2.78 (m, 1H) 2.38-2.51 (m, 2H) 2.26-2.34 (m, 2H) 1.90-1.97 (m, 1H) 1.82 (q, J=11.55 Hz, 1H) 1.52-1.68 (m, 5H) 1.28-1.38 (m, 1H) 1.01 (d, J=7.17 Hz, 3H) 0.92 (s, 3H).

### Example 38

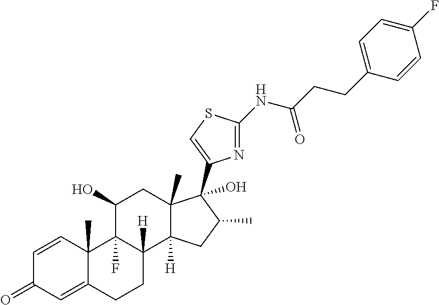

Prepared using method C. Purified by flash chromatography with 30-50% EtOAc:heptanes. Yield=34 mg (51%), white solid. LCMS (7 minute method): 4.84 min (583; M+H). HRMS 583.2448 (expected for C₃₂H₃₆F₂N₂O₄S 583.2442).

¹H NMR (500 MHz, MeOD) δ ppm 7.42 (d, J=10.07 Hz, 1H) 7.22-7.28 (m, 2H) 6.96-7.03 (m, 2H) 6.87 (s, 1H) 6.28 (dd, J=10.07, 1.83 Hz, 1H) 6.08 (s, 1H) 4.20-4.26 (m, 1H) 3.07-3.14 (m, 1H) 2.97-3.03 (m, 2H) 2.68-2.77 (m, 3H) 2.35-2.50 (m, 2H) 2.22-2.32 (m, 2H) 1.88-1.96 (m, 1H) 1.74-1.84 (m, 1H) 1.50-1.61 (m, 5H) 1.26-1.34 (m, 1H) 0.97 (d, J=7.17 Hz, 3H) 0.88 (s, 3H).

### Example 39

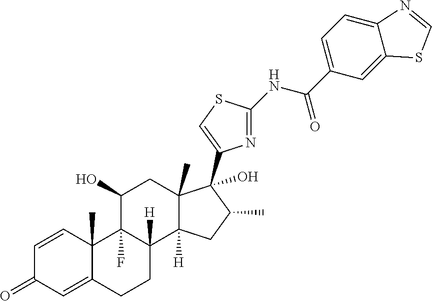

Prepared using method B. Purified by flash chromatography with 50-80% EtOAc:heptanes. Resulting solid triturated in EtOAc, filtered and dried. Yield=20 mg (29%), white solid. LCMS (7 minute method): 4.59 min (594; M+H). HRMS 594.1891 (expected for C₃₁H₃₂FN₃O₄S₂ 594.1897).

¹H NMR (500 MHz, DMSO-d₆) δ ppm 12.72 (br. s., 1H) 9.60 (s, 1H) 8.95 (d, J=0.92 Hz, 1H) 8.18-8.26 (m, 2H) 7.30 (d, J=10.07 Hz, 1H) 7.04 (s, 1H) 6.23 (dd, J=10.15, 1.75 Hz, 1H) 6.02 (s, 1H) 5.13 (d, J=1.68 Hz, 1H) 4.38 (br. s., 1H) 4.15 (dd, J=10.91, 0.99 Hz, 1H) 3.11-3.20 (m, 1H) 2.59-2.68 (m, 1H) 2.28-2.42 (m, 2H) 2.15-2.25 (m, 2H) 1.78-1.86 (m, 1H) 1.73 (q, J=11.34 Hz, 1H) 1.47-1.55 (m, 4 H) 1.39 (qd, J=12.77, 4.73 Hz, 1H) 1.19-1.27 (m, 1H) 0.91 (d, J=7.02 Hz, 3H) 0.84 (s, 3H).

### Example 40

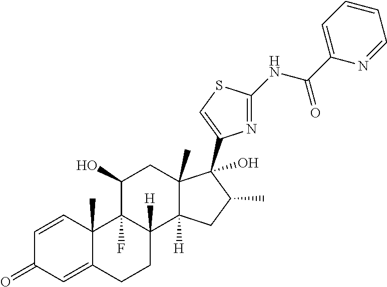

Prepared using method B. Purified by flash chromatography with 30-50% EtOAc:heptanes. Yield=61 mg (98%), white solid. LCMS (7 minute method): 4.67 min (538; M+H). HRMS 538.2192 (expected for C₂₉H₃₂FN₃O₄S 538.2176).

¹H NMR (500 MHz, MeOD) δ ppm 8.73-8.78 (m, 1H) 8.25 (d, J=7.78 Hz, 1H) 8.05 (td, J=7.74, 1.60 Hz, 1H) 7.66 (ddd, J=7.63, 4.73, 1.07 Hz, 1H) 7.44 (d, J=10.07 Hz, 1H) 7.01 (s, 1H) 6.29 (dd, J=10.07, 1.98 Hz, 1H) 6.09 (s, 1H) 4.27 (ddd, J=11.14, 3.74, 1.75 Hz, 1H) 3.07-3.16 (m, 1H) 2.69-2.79 (m, 1H) 2.38-2.52 (m, 2H) 2.27-2.37 (m, 2H) 1.90-1.98 (m, 1H) 1.78-1.89 (m, 1H) 1.67 (dd, J=14.19, 1.68 Hz, 1H) 1.52-1.64 (m, 4H) 1.30-1.38 (m, 1H) 1.03 (d, J=7.17 Hz, 3H) 0.91 (s, 3H).

### Example 41

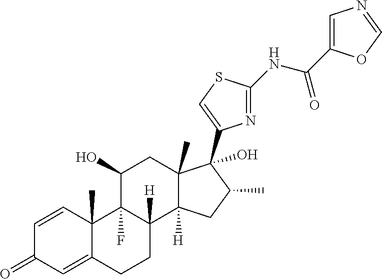

Prepared using method B. Purified by flash chromatography with 50-70% EtOAc:heptanes. Yield=35 mg (57%), white solid. LCMS (7 minute method): 4.13 min (528; M+H). HRMS 528.1970 (expected for C₂₇H₃₀FN₃O₅S 528.1968).

¹H NMR (500 MHz, DMSO-d₆) δ ppm 12.87 (br. s., 1H) 8.69 (s, 1H) 8.26 (br. s., 1H) 7.29 (d, J=10.22 Hz, 1H) 7.04 (s, 1H) 6.22 (dd, J=10.07, 1.83 Hz, 1H) 6.01 (s, 1H) 5.12 (dd, J=3.36, 1.83 Hz, 1H) 4.40 (br. s., 1H) 4.10-4.18 (m, 1H) 3.12 (br. s., 1H) 2.63 (td, J=13.24, 6.03 Hz, 1H) 2.26-2.41 (m, 2H) 2.14-2.24 (m, 2H) 2.08 (s, 1H) 1.77-1.85 (m, 1H) 1.71 (q, J=11.39 Hz, 1H) 1.48 (s, 3H) 1.33-1.44 (m, 1H) 1.22-1.29 (m, 1H) 0.90 (d, J=7.17 Hz, 3H) 0.81 (s, 3H).

### Example 42

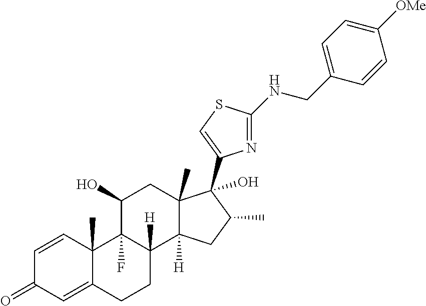

Mesylated dexamethasone (50 mg, 0.11 mmol), N-4-methoxybenzylthiourea (25 mg, 0.13 mmol) and dimethylaniline (54 μL, 0.43 mmol) in MeCN (1 mL) were heated in a sealed tube at 100° C for 4 h. Product crystallised out of the reaction mixture. Solid was filtered, washed with MeCN and dried. Yield=45 mg (74%), white solid. LCMS (7 minute method): 3.87 min (553; M+H).

¹H NMR (500 MHz, MeOD) δ ppm 7.41 (d, J=10.07 Hz, 1H) 7.31-7.36 (m, 2H) 6.93-6.98 (m, 2H) 6.70 (s, 1H) 6.29 (dd, J=10.15, 1.91 Hz, 1H) 6.09 (s, 1H) 4.48-4.57 (m, 2H) 4.28 (ddd, J=10.57, 3.70, 1.91 Hz, 1H) 3.80 (s, 3H) 2.68-2.84 (m, 5 H) 2.35-2.52 (m, 3H) 2.28 (td, J=11.63, 8.47 Hz, 1H) 1.88-1.95 (m, 1H) 1.82 (q, J=11.65 Hz, 1H) 1.48-1.61 (m, 4H) 1.41 (dd, J=13.81, 1.45 Hz, 1H) 1.27-1.34 (m, 1H) 0.96-1.01 (m, 6H)

### Example 43

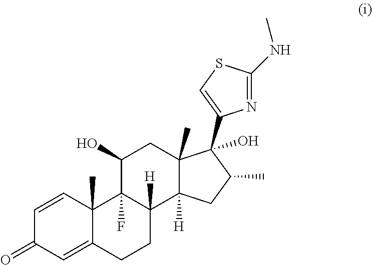

Mesylated dexamethasone (200 mg, 0.43 mmol) and N-methylthiourea (46 mg, 0.51 mmol) in pyridine (2 mL) were heated to 80° C for 2 h. Reaction was concentrated, water added and extracted with EtOAc. Organics were separated, dried over Na₂SO₄ and concentrated. Product was purified by flash chromatography with 60% EtOAc:heptanes. Yield=68 mg (35%), white solid. LCMS (7 minute method): 3.13 min (447; M+H). HRMS 447.2110 (expected for C₂₄H₃₁FN₂O₃S 447.2118). ¹H NMR (500 MHz, DMSO-d₆) δ ppm 7.35 (q, J=4.58 Hz, 1H) 7.29 (d, J=10.07 Hz, 1H) 6.36 (s, 1H) 6.21 (dd, J=10.07, 1.83 Hz, 1H) 6.00 (s, 1H) 5.09 (dd, J=3.59, 1.91 Hz, 1H) 4.07-4.15 (m, 1H) 3.94 (s, 1H) 2.84-2.93 (m, 1H) 2.77 (d, J=4.88 Hz, 3H) 2.57-2.67 (m, 1H) 2.23-2.39 (m, 2H) 2.03-2.16 (m, 2H) 1.75-1.84 (m, 1H) 1.65 (q, J=11.65 Hz, 1H) 1.51-1.57 (m, 1H) 1.48 (s, 3H) 1.37 (qd, J=12.74, 5.11 Hz, 1H) 1.09-1.15 (m, 1H) 0.87 (d, J=7.17 Hz, 3H) 0.83 (s, 3H).

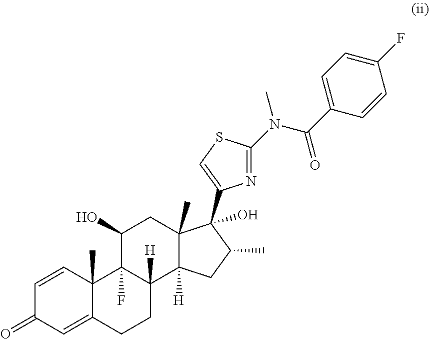

Prepared using method A as above using the aminothiazole produced in step (i). Product was purified by flash chromatography with 25-35% EtOAc:heptanes. Yield=44 mg (68%), white solid. LCMS (7 minute method): 4.96 min (569; M+H). HRMS 569.2288 (expected for C₃₁H₃₄F₂N₂O₄S569.2286).

¹H NMR (500 MHz, CDCl₃) δ ppm 7.55-7.62 (m, 2H) 7.15-7.25 (m, 3H) 6.81 (s, 1H) 6.29 (dd, J=10.15, 1.75 Hz, 1H) 6.11 (s, 1H) 4.36 (dd, J=6.33, 3.89 Hz, 1H) 3.65 (s, 3H) 3.01 (s, 1H) 2.86-2.96 (m, 1H) 2.58-2.68 (m, 1H) 2.28-2.45 (m, 4 H) 1.83-1.91 (m, 1H) 1.68-1.82 (m, 2H) 1.57-1.68 (m, 1H) 1.54 (s, 3H) 1.47 (d, J=14.19 Hz, 1H) 1.40 (ddd, J=12.21, 7.71, 4.04 Hz, 1H) 1.03 (d, J=7.17 Hz, 3H) 0.91 (s, 3H).

### Example 44

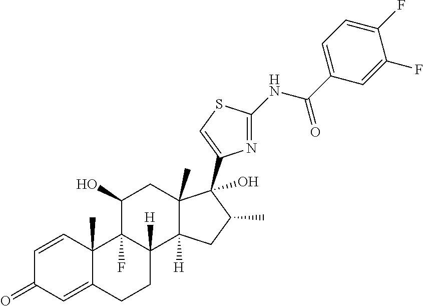

Prepared using method A. Purified by flash chromatography with 25% EtOAc:heptanes. Yield=61 mg (89%), white solid. LCMS (7 minute method): 4.83 min (573; M+H). HRMS 573.2046 (expected for C₃₀H₃₁F₃N₂O₄S 573.2035).

¹H NMR (500 MHz, MeOD) δ ppm 7.94-8.03 (m, 1H) 7.90 (d, J=7.63 Hz, 1H) 7.39-7.52 (m, 2H) 6.96 (s, 1H) 6.29 (dd, J=10.07, 1.68 Hz, 1H) 6.09 (s, 1H) 4.26 (dd, J=10.99, 1.68 Hz, 1H) 3.08-3.19 (m, 1H) 2.73 (td, J=13.12, 6.10 Hz, 1H) 2.37-2.53 (m, 2H) 2.25-2.36 (m, 2H) 1.89-1.98 (m, 1H) 1.83 (q, J=11.19 Hz, 1H) 1.51-1.66 (m, 5 H) 1.33 (ddd, J=12.21, 8.24, 4.12 Hz, 1H) 1.01 (d, J=7.17 Hz, 3H) 0.92 (s, 3H).

### Example 45

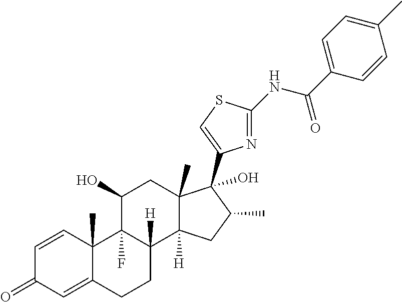

Prepared using method A. Purified by flash chromatography with 10-20% EtOAc:heptanes. Yield=44 mg (67%), white solid. LCMS (7 minute method): 4.81 min (551; M+H). HRMS 551.2362 (expected for C₃₁H₃₅FN₂O₄S 551.2380).

¹H NMR (500 MHz, MeOD) δ ppm 7.91 (d, J=8.09 Hz, 2H) 7.43 (d, J=10.07 Hz, 1H) 7.37 (d, J=7.93 Hz, 2H) 6.94 (s, 1H) 6.29 (dd, J=10.07, 1.68 Hz, 1H) 6.09 (s, 1H) 4.22-4.30 (m, 1H) 3.08-3.16 (m, 1H) 2.73 (td, J=13.31, 5.72 Hz, 1H) 2.36-2.52 (m, 5 H) 2.25-2.36 (m, 2H) 1.89-1.98 (m, 1H) 1.82 (q, J=11.49 Hz, 1H) 1.51-1.69 (m, 5 H) 1.30-1.37 (m, 1H) 1.01 (d, J=7.17 Hz, 3H) 0.92 (s, 3H).

### Example 46

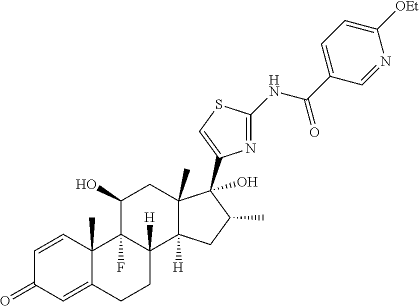

Prepared using method A. Purified by flash chromatography with 10-20% EtOAc:heptanes. Yield=37 mg (53%), white solid. LCMS (7 minute method): 4.83 min (581; M+H). HRMS 581.2498 (expected for C₃₂H₃₇FN₂O₅S 581.2485).

¹H NMR (500 MHz, MeOD) δ ppm 7.98 (d, J=8.70 Hz, 2H) 7.43 (d, J=10.07 Hz, 1H) 7.04 (d, J=8.70 Hz, 2H) 6.91 (s, 1H) 6.29 (dd, J=10.07, 1.53 Hz, 1H) 6.09 (s, 1H) 4.26 (dd, J=11.06, 1.75 Hz, 1H) 4.13 (q, J=7.02 Hz, 2H) 3.05-3.16 (m, 1H) 2.72 (td, J=13.47, 6.03 Hz, 1H) 2.35-2.51 (m, 2H) 2.25-2.35 (m, 2H) 1.88-1.96 (m, 1H) 1.81 (q, J=11.49 Hz, 1H) 1.64 (d, J=14.19 Hz, 1H) 1.49-1.61 (m, 4 H) 1.42 (t, J=6.94 Hz, 3H) 1.27-1.36 (m, 1H) 1.01 (d, J=7.17 Hz, 3H) 0.91 (s, 3H).

### Example 47

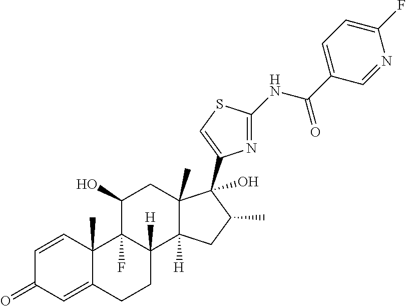

Prepared using method B. Purified by flash chromatography with 30% EtOAc:heptanes. Yield=34 mg (51%), white solid. LCMS (7 minute method): 4.43 min (556; M+H).

¹H NMR (500 MHz, MeOD) δ ppm 8.87 (br. s., 1H) 8.54 (t, J=7.02 Hz, 1H) 7.43 (d, J=10.07 Hz, 1H) 7.24 (dd, J=8.54, 1.53 Hz, 1H) 6.97 (br. s., 1H) 6.29 (dd, J=10.15, 1.75 Hz, 1H) 6.09 (s, 1H) 4.26 (dd, J=11.06, 1.60 Hz, 1H) 3.09-3.22 (m, 1H) 2.73 (td, J=13.35, 5.80 Hz, 1H) 2.37-2.53 (m, 2H) 2.25-2.36 (m, 2H) 1.88-1.98 (m, 1H) 1.82 (q, J=11.34 Hz, 1H) 1.50-1.66 (m, 5H) 1.25-1.37 (m, 1H) 1.01 (d, J=7.17 Hz, 3H) 0.92 (s, 3H).

### Biological Examples

General Methods

Abbreviations:

DMEM—Dulbecco's modified Eagle medium.

FBS—Foetal bovine serum.

PBS—phosphate buffered saline.

PLB—passive lysis buffer.

Materials and Methods:

Foetal bovine serum (FBS) and Opti-MEM™ I reduced serum media were obtained from Invitrogen. Dulbecco's modified Eagle medium (DMEM) and stable glutamine solution were obtained from PAA Laboratories GmbH (Pasching, Austria). DMSO was obtained from Sigma-Aldrich.

The NFκB reporter gene plasmid pNF-κB Luc, with five NFKB response elements (TGGGGACTTTCCGC)5, was obtained from Stratagene. The plasmid pGL4-hRLuc encoding the Renilla luciferase was obtained from Promega.

FuGENE® HD Transfection Reagent was obtained from Roche. The Dual Luciferase Reporter® system and Passive Lysis Buffer (PLB) was obtained from Promega Corporation.

HeLa cells were obtained from ECACC, and propagated in DMEM containing 10% FBS. Cells were routinely incubated in 37° C and 5% CO₂.

Compounds:

Compounds were solubilized in DMSO (5 mM), and then further diluted to the required concentration with DMEM.

Transactivation Assay:

HeLa cells were seeded at 80×10⁴ cells/dish in 100 mm dishes. Cells were transfected the next day with MMTV-Luc (5 μg) and pGL4-hRLuc (0.1 μg), in 250 μL of Opti-MEM I using FuGENE HD (15 μL). After 24 hours, cells were trypsinized and seeded into 96-well plates at a density of 8-10×10⁴ cells/mL.

Following overnight incubation to allow attachment, cells were treated with the required concentration of compounds, then further incubated for 16 hours. Media was aspirated and cells lysed in 40 μL of 1×PLB.

Firefly and Renilla luciferase activity was measured using the Dual Luciferase Reporter system following the manufacturer's protocol.

Transrepression Assay:

HeLa cells were seeded at 80×10⁴ cells/dish in 100 mm dishes. Cells were transfected the next day with NFκB-LUC reporter (5 μg) and pGL4-hRLuc (0.1 μg), in 0.25 mL of Opti-MEM I using FuGENE HD (15 μL). After 24 hours, cells were trypsinized and seeded in to 24-well plates at a density of 8-10×10⁴ cells/mL. Following overnight incubation, cells were starved for a further 24 hours in DMEM.

On the day of the experiment, compounds were added to cells one hour prior to the addition of TNFκ (0.5 ng/mL). After a further 16 hours, cells were washed twice with ice-cold PBS, then lysed with 100 μL of 1×PLB.

Firefly and Renilla luciferase activity was measured using the Dual Luciferase Reporter system following the manufacturer's protocol.

IC50 and EC50:

IC50 and EC50 values were calculated by using GraphPad Prism 5.0. Briefly, luminescence values for luciferase were normalised using the Renilla luminescence values. The normalised values were input into GraphPad Prism 5.0. Non-linear regression analysis was carried out using the “log(concentration) vs. response” equation of the software, with a Hill slope coefficient fixed at 1.

IC50 and EC50 values are mean values in nM of all IC50/EC50 values for the compound. Max % values are mean values of the Max% activity.

Results

| | |
|---|---|
| TA | TR |

| Compound | EC50 | Max % | IC50 | Max % |
|---|---|---|---|---|
| 1 | 867.88 | 21.33 | 8.41 | 90.81 |
| 2 | 1177.27 | 18.28 | 23.48 | 52.36 |
| 3 | 1154 | 24.14 | 21.90 | 76.03 |
| 4 | 69.24 | 39.44 | 3.95 | 105.75 |
| 5 | 320.85 | 27.19 | 8.13 | 103.93 |
| 6 | 890.07 | 16.02 | 11.51 | 84.56 |
| 7 | 293.65 | 43.95 | 19.35 | 97.33 |
| 8 | 247.95 | 49.73 | 5.69 | 95.04 |
| 9 | 1109.85 | 15.14 | 25.03 | 80.48 |
| 10 | 3392 | 4.79 | 35.38 | 55.14 |
| 11 | 113.99 | 36.5 | 30.22 | 88.85 |
| 12 | 718.8 | 23.75 | 40.69 | 91.11 |
| 13 | 188.86 | 30.02 | 10.82 | 87.58 |
| 14 | 535.97 | 36.56 | 23.93 | 88.59 |
| 15 | 1226.5 | 20.83 | 3.53 | 79.73 |
| 16 | 881 | 21.19 | 26.75 | 87.25 |
| 17 | 831.8 | 39.10 | 1.19 | 82.30 |
| 18 | 668.8 | 26.29 | 6.83 | 82.12 |
| 19 | 406450 | 0.84 | 1451 | 33.32 |
| 23 | 1401.82 | 5.67 | 35.82 | 52.14 |
| 24 | 7692.33 | 2.63 | 5.96 | 78.99 |
| 25 | 3929 | 1.92 | 29.05 | 79.05 |
| 26 | 1534.5 | 3.9 | 8.22 | 44.09 |
| 27 | 1640 | 5.89 | 10.95 | 49.67 |
| 28 | 29350 | 0 | 119.6 | 36.02 |
| 29 | 19380 | 0.67 | 7.96 | 44.18 |
| 30 | 1414.5 | 7.43 | 35.55 | 41.34 |
| 31 | — | 0 | 11.25 | 37.49 |
| 32 | 2233 | 0.03 | 55.21 | 28.52 |
| 33 | — | 0 | 66.86 | 32.70 |
| 34 | 17550 | 0 | 4.76 | 51.77 |
| 35 | 4176 | 0.23 | 44.77 | 35.86 |
| 36 | 5520 | 1.19 | 10.61 | 33.64 |
| 37 | 2767 | 3.22 | 17.86 | 31.44 |
| 38 | 24400 | 2.48 | 31.24 | 65.45 |
| 39 | 1155 | 0.96 | 8.34 | 19 |
| 40 | 16936.5 | 1.34 | 8.34 | 19 |
| 41 | 10338 | 1.27 | 41.75 | 26.025 |
| 42 | 391.5 | 0.56 | 198.81 | 51.08 |
| 43 | 86200 | 1.63 | 154.70 | 61.01 |
| 44 | 914.1 | 14.64 | 28.63 | 41.79 |
| 45 | 8479 | 5.74 | 20.19 | 42.28 |
| 46 | 6650.5 | 9.95 | 27.04 | 55.13 |
| 47 | 669.4 | 7.41 | 22.9 | 48.04 |

The IC50 is the concentration required for half-maximal repression of NFkB, TR Max % is the comparison of maximal NFkB repression compared to Dexamethasone, EC50 is the concentration for half-maximal transactivation, and TA Max % is the comparison against maximal activation seen with dexamethasone.

Comparison Studies:

Comparisons were made against conventional, therapeutic steroids, dexamethasone, and prednisolone. The EC50 and maximal effects were measured and compared to conventional steroids. The table below summarises the results of multiple experiments comparing conventional steroids dexamethasone (Dex) and prednisolone (Pred) against novel substituted steroids of the invention (compounds). The IC50 is the concentration required for half-maximal repression of NFkB, TR % is the comparison of maximal NFkB repression compared to Dexamethasone, EC50 is the concentration for half-maximal transactivation, and TA % is the comparison against maximal activation seen with dexamethasone.

| Compound | IC50 | TR % | EC50 | TA % |
|---|---|---|---|---|
| Dex | 2.86 | 100 | 6.78 | 100 |
| Pred | 12.86 | 99 | 238.18 | 68 |
| 7 | 1.22 | 98 | 293.65 | 44 |
| 17 | 1.19 | 87 | 831.80 | 39 |
| 18 | 5.96 | 84 | 668.80 | 26 |
| 23 | 35.82 | 52 | 1401.82 | 6 |

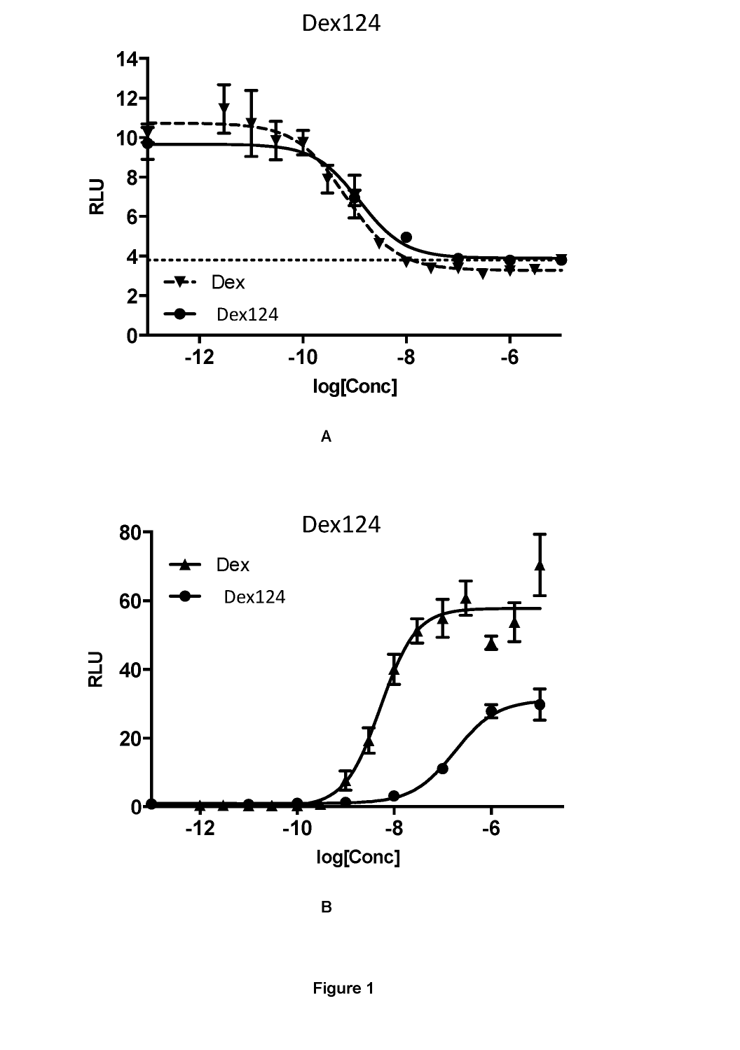

FIG. 1A shows the results of head-to-head comparison between the synthetic, conventional glucocorticoid dexamethasone and an exemplary novel substituted steroid of the present invention, referred to as Dex124, showing similar dose-response curves for repression of NFkB activity. FIG. 1B shows a marked reduction in maximal transactivation, with a less notable right-shift in the dose-response curve.

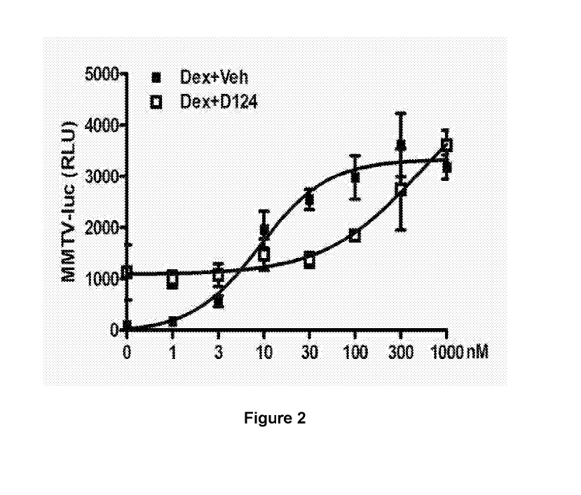

Transactivation reporter cells with a fixed concentration of Dex124, or vehicle, were subjected to a dose-response of dexamethasone. FIG. 2 shows basal transactivation with Dex124 but also that the dose-response to dexamethasone was right-shifted, supporting competitive antagonism. This data supports the idea that Dex124 is a high affinity, partial agonist for the GR.

There is a high degree of structural similarity between the various human steroid receptors. Ligands may therefore cross-react with other members of the steroid receptor family. In particular progesterone receptor and mineralocorticoid receptor show the highest structural similarity. The activity of Dex124 was compared to the conventional steroid prednisolone, and the cognate ligand for each of the progesterone receptor (PR), androgen receptor (AR), and mineralocorticoid receptor (MR).

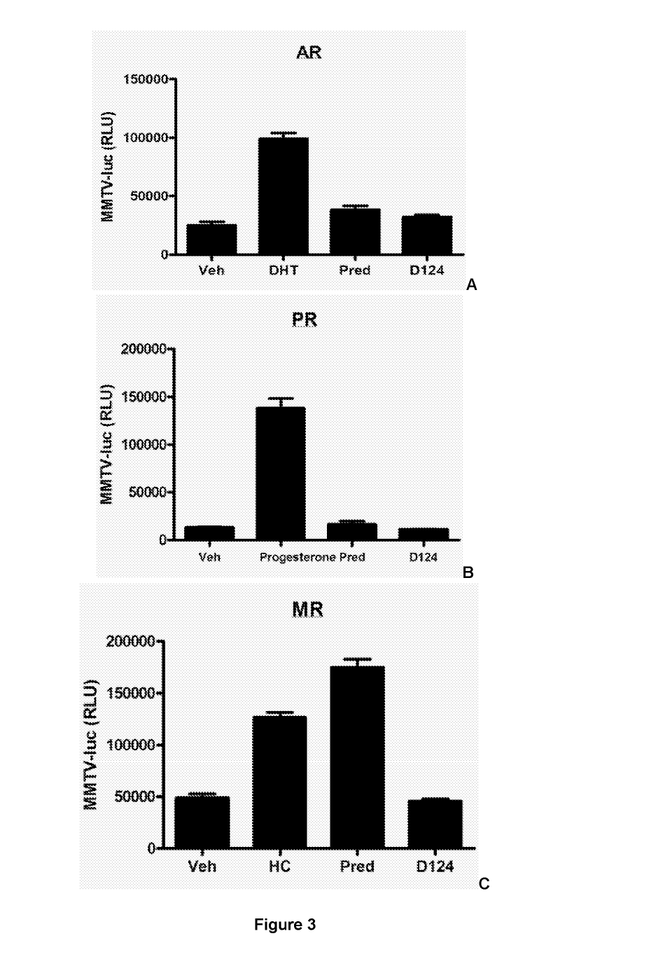

Results are shown in FIG. 3. Dex124 (D124) shows no activity on any of the three steroid receptors. However, prednisolone has significant activity on the mineralocorticoid receptor, and therefore is predicted to result in salt and water retention, and accelerated cardiovascular risk in humans, as is indeed observed in clinical studies. The sparing of MR activation seen with Dex124 is an unexpected additional therapeutic advantage.

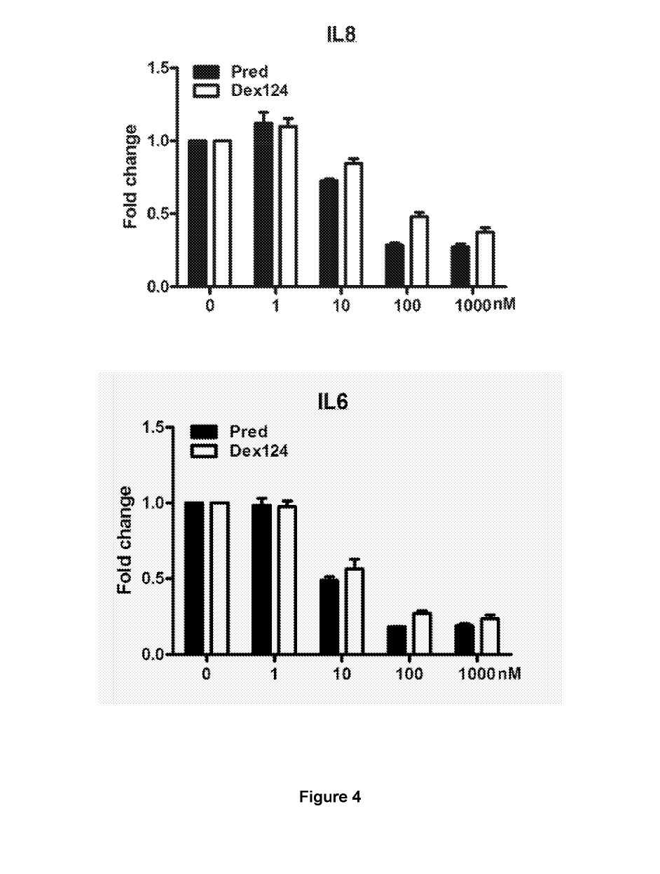

Repression of pro-inflammatory cytokine expression was documented as an additional means to assess potential anti-inflammatory action. Briefly, these studies measure steroid-induced suppression of pro-inflammatory cytokine release from cells in culture after stimulation with the pro-inflammatory cytokine TNFα, a major effector of disease manifestation in rheumatoid arthritis. FIG. 4 summarises the results and shows that Dex124 shows comparable efficacy and potency to the conventional steroid prednisolone.

Off-Target Effects:

In-Vivo Anti Rheumatic

Established rat arthritis was chosen as the most relevant predictive model for chronic inflammatory arthritis in man. The disease is initiated with a caudal injection of complete Freund's adjuvant and the disease is established after 14 days. Anti-inflammatory molecules are given orally, usually once a day and after 7 days the animals are sacrificed. Paw volumes are the primary measurement and as a secondary measure inflammatory lesions are scored at a number of sites. At autopsy adrenal, spleen and thymus weights are recorded.

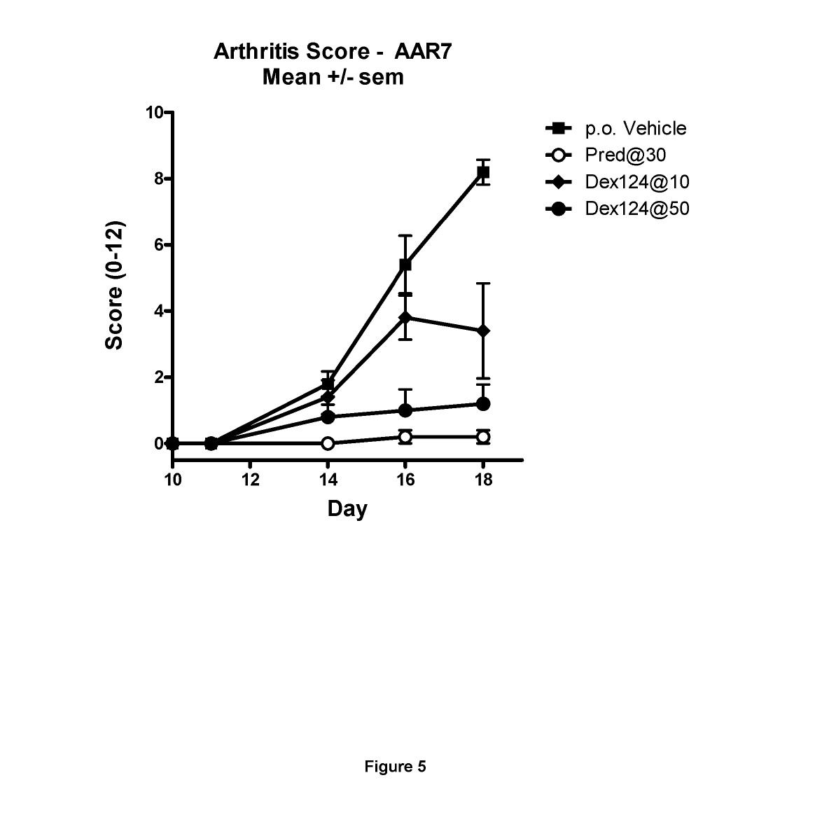

Dex124 was tested over a wide range of doses from 0.5 to 100 mg/kg daily for seven days and the data showed a consistent dose dependent reduction in paw volumes and symptoms although with some variation between assays. FIG. 5 shows dose-dependent inhibition of rat adjuvant arthritis with Dex124, and comparison with prednisolone at a single dose.

The arthritis score for the rats was used in these studies, as the inflammatory disease consists of both destructive inflammatory arthritis, but also systemic manifestations. This data provided strong support for the anti-rheumatic activity of Dex124.

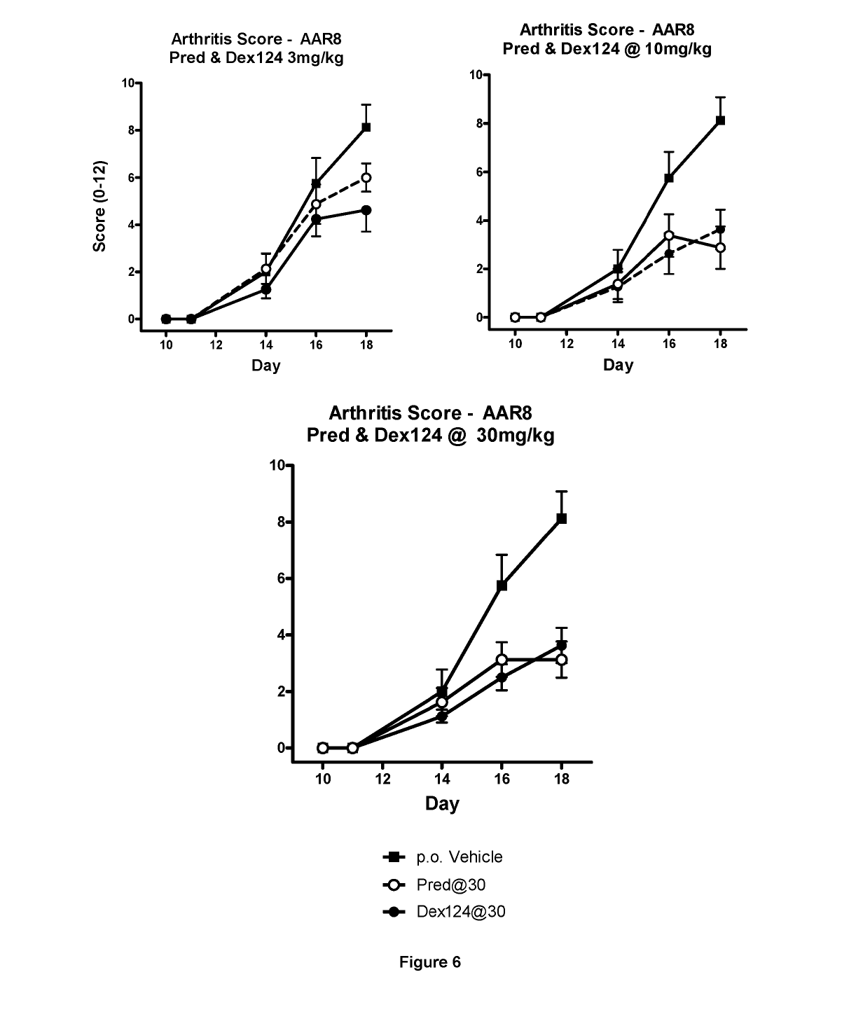

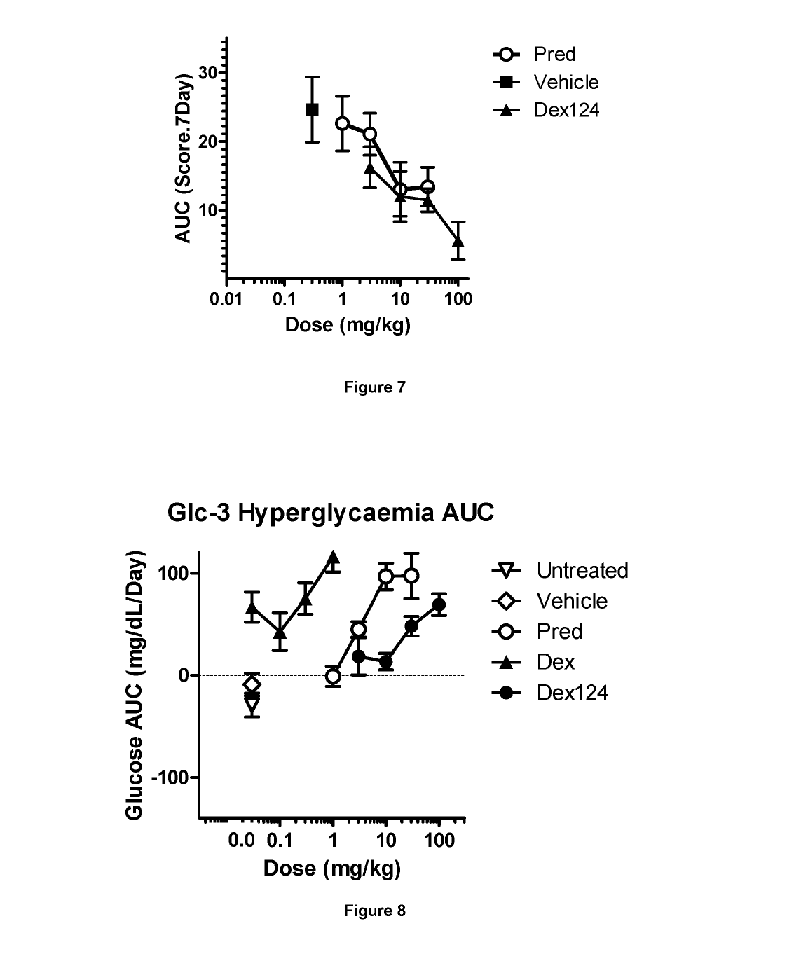

More comprehensive head-to-head comparisons between Dex124, and prednisolone were then undertaken. The three graphs in FIG. 6 show the evolution of arthritis in response to prednisolone or Dex124. The head-to-head comparison was extended to measurement of the clinical score, as used above, and all the data are summarised in FIG. 7, which shows the clinical score by steroid, and by dosage. This analysis uses the area under the curve of arthritis score over the course of the experiment to yield a single, integrated measure for each experimental animal. The area under the curve of the vehicle treated animal is indicated on the graph for comparison purposes, but marked at an arbitrary log dose. Increasing doses of prednisolone, and Dex124 both inhibit arthritis clinical score, as indicated by a decline in the area under curve total (AUC). This analysis reveals similar efficacy and potency for Dex124 and prednisolone in the rat adjuvant arthritis study.

In-Vivo Glucose Metabolism

Further in-vivo studies used glucose homeostasis, which is a useful surrogate for the energy metabolic disruption seen with steroid use in humans. The acute induction of serum glucose in the fasting rat in response to a single dose of steroid was used as a simple and robust measure of disruption to glucose homeostasis. The induction was tracked over time, and the area under the curve, compared to vehicle, is taken as the integrated effect of steroid administration.

FIG. 8 shows induction of serum glucose in response to dexamethasone (Dex), prednisolone (Pred) and Dex124. The response to GC administration is an acute rise in serum glucose which can be plotted over time. The area under the response curve can be plotted to offer an integrated metabolic response. The untreated animals and vehicle-treated animals show a negative area, as the serum glucose falls over time in these animals resulting in an area under the curve which is below the baseline and so is plotted as a negative value. Both vehicle and untreated groups are assigned an arbitrary dose value to permit plotting on the log scale.

The dose-response curve for Dex124 is right-shifted, and the maximal effect lower than that for prednisolone, which means that glucose homeostasis is resistant to Dex124. This suggests that Dex124 will have less effect on disturbing glucose and energy metabolism than prednisolone.

In-Vivo Bone Metabolism

For the analysis of bone metabolism, and its regulation by orally administered GC, a rat bone formation assay was used. This was to permit direct comparison between the different steroid actions in the same species and also to model the human pathology, in which it is now clear that long-term steroid use primarily affects osteoblast function and so limits bone remodelling, leading to a progressive loss of bone mass. It also leads, importantly, to a loss of structural bone strength, as loss of osteoblast action results in impaired bone repair and accumulation of microfractures.

Osteocalcin Analysis

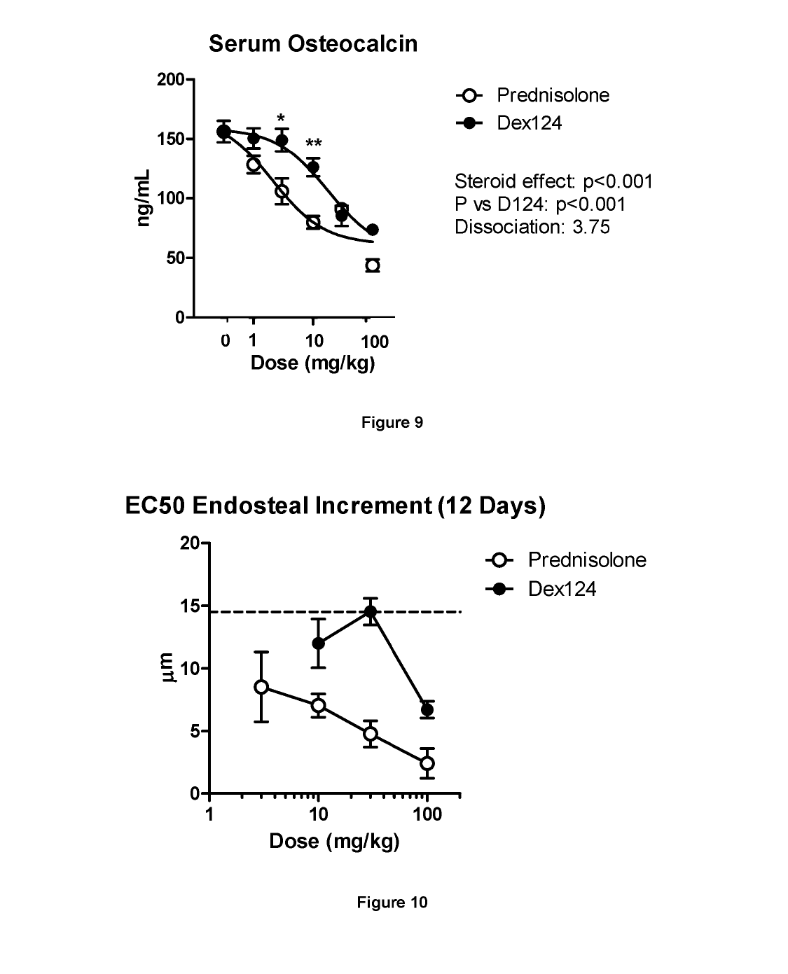

Serum osteocalcin is a convenient circulating biomarker of osteoblast activity, and has previously been shown to be suppressed in response to steroid administration. Rats were treated with daily, oral administration of prednisolone or Dex124 in varying doses to permit the acquisition of dose-response curves. Terminal (35 day) plasma was harvested, and osteocalcin measured, as an integrated measure of osteoblast activity across the skeleton. This revealed a dose-dependent inhibition of osteocalcin concentration with prednisolone, and a right-shifted dose response for Dex124 (see FIG. 9). This right-shift again suggests that Dex124 has less effect on osteoblast function in-vivo than prednisolone. Vehicle had no effect on serum osteocalcin, and so was not plotted. Calculated IC50 values suggest a right-shift in IC₅₀ of at least three fold for Dex124, compared to prednisolone. This indicates a selectivity of action for Dex124 for the anti-inflammatory and anti-rheumatic actions as opposed to the off-target bone effects of steroids.

Endosteal Bone Increment

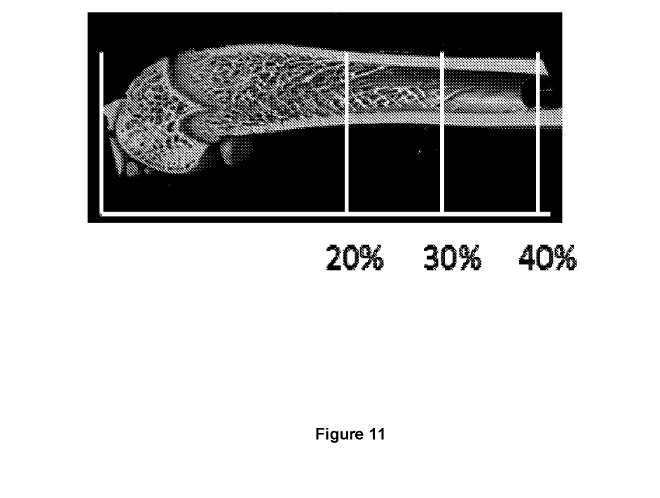

The osteocalcin data suggested a differential effect on osteoblast function between the two steroids. However, osteoblast activity is not uniform across the skeleton, which may result in either over, or under estimation of the steroid effect. In young, growing, rats long bone remodelling during bone growth offers an attractive model to study active bone formation as the endosteal surface has new bone formation as the diameter of the shaft decreases due to endosteal bone formation, and periosteal bone resorption. As shown in FIG. 11 (CT reconstruction) as long bones grow the diameter of the shaft at the growing end (20% mark) is greater than that towards the middle of the bone (30% and 40% marks) due to loss of bone at the outer surface, or periosteal surface, and deposition of bone, due to osteoblast action, on the inner surface, or endosteal surface.

As shown in FIG. 10 there was a marked dose-dependent decrease in bone increment seen with prednisolone, with a calculated IC50 of 7.8 mg/Kg. In contrast, there was no effect of Dex124 seen until 100 mg/Kg, giving an estimated IC50 of approximately 90 mg/Kg. Taken together, these data show that Dex124 exerts a markedly reduced effect on bone formation, and osteoblast function compared to prednisolone. The dose-response curve is right-shifted, indicating that bone effects with Dex124 require higher doses. Hence prednisolone and Dex124, when administered at the same daily dose, with equivalent anti-rheumatic activity, will exert different effects on bone with Dex124 being bone-sparing.

Micro Computer Tomography

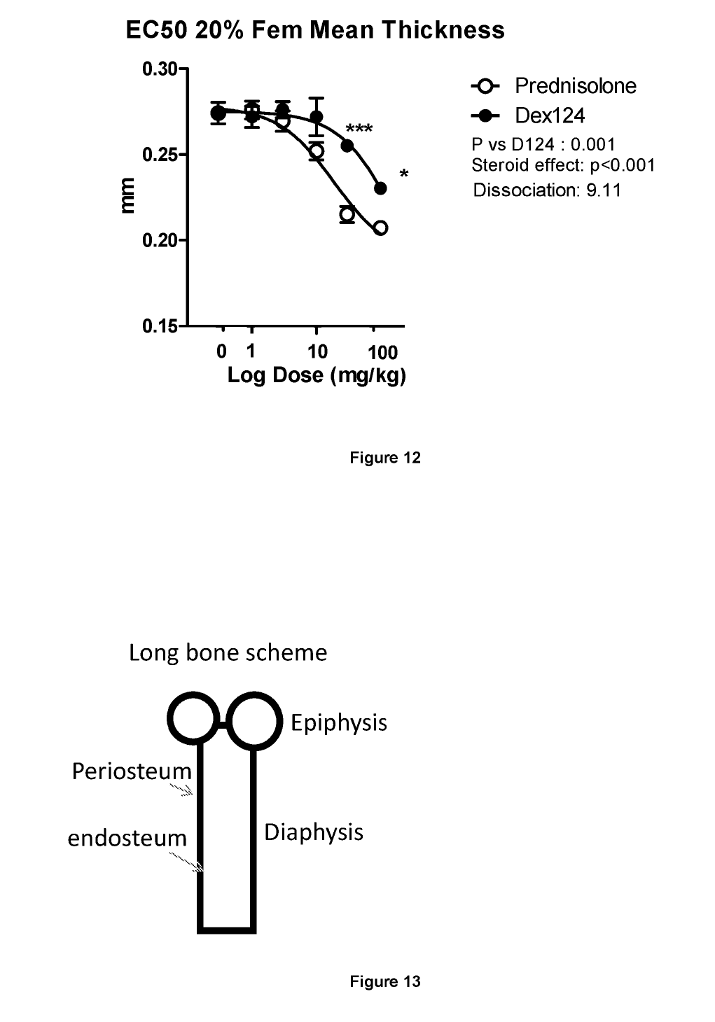

This imaging modality offers a non-destructive means to measure bone structure and mineral content. Analysis was performed at defined points in relation to the total bone length, as steroid dosed bones were overall shorter than controls. The major effect of prednisolone was seen at 20%, where the shaft was undergoing modelling and was at its thinnest. At this point there was a significant difference seen between prednisolone and Dex124, with a 9.1 fold increase in IC50 measured. This again supports a marked reduction in bone disruption caused by Dex124 compared to prednisolone (FIG. 12).

Further analysis of the trabecular bone in the femoral head was performed. FIG. 13 shows the epiphysis, or end, of the long bone which consists of medial and lateral condyles, the rounded protuberances. The bone density of the fermoral condyles in response to prednisolone or Dex124 was analysed. This data revealed a surprising selectivity of steroid action between the lateral, and medial condyles, but also found a significant bone sparing effect with Dex124.

Safety Studies:

Pharmacokinetic, metabolic and safety studies were carried out on an exemplary molecule of the invention, Dex124, and suggest that is a benign molecule able to be administered orally for 14 days at up to 150 mg/kg with a no effect level at 15 mg/kg/day over this period. It carries no risk of mutagenic or cardiac arrhythmia problems at concentrations far in excess of those used in the in vitro studies for efficacy, and also far in excess of the concentrations seen following oral administration in the PK, and toxicoPK studies. The serum concentrations seen in the PK studies are well below the no effect level seen in the safety studies. The ED50 dose of Dex124 for beneficial effects on arthritis was almost 5 times less than the dose threshold for no effect in the safety studies. Even at 150 mg/kg for 14 days the only changes seen were related to weight loss and no compound related organ toxicity was reported.

1. A compound of formula (II): 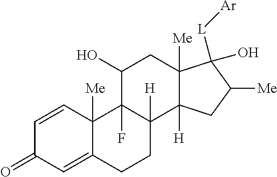
wherein L is a linker group selected from L¹ and L²; and
Ar is selected from phenyl and Cs.yheteroaryl, optionally substituted with one or
more substituents R^{A};
wherein: each R^{A} is independently selected from: —F, —Cl, —Br, —I, —OR^{O}, —N(R^{N})₂, —C(═O)OR^{O}, —C(═O)N(R^{N})₂, —SO₂N(R^{N})₂, —CF₃, and —CN, wherein each R^{O} and R^{N} is independently selected from —H and —C₁₋₄ alkyl;
L¹ is: 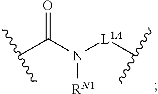
L² is: 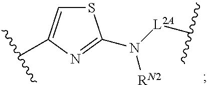
L^{1A} is selected from -L^{A}- and -L^{B}-O—;
L^{2A} is selected from —C(═O)—, —C(═O)-L^{B}-, and -L^{B}-;
wherein L^{A} is saturated C₃₋₄ alkylene and L^{B} is saturated C₁₋₄ alkylene;
and R^{N1}and R^{N2} are each independently selected from —H and -Me.

2. The compound of claim 1, which is a compound of formula (IIa): 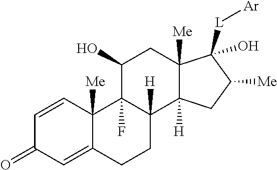
wherein L and Ar are as previously defined.

3. The compound of claim 1, wherein Ar is independently selected from phenyl, pyridinyl, thiazolyl, pyrrolyl, furanyl, benzothiazolyl, and benzoxazolyl, optionally substituted with one or more substituents R^{A}.

4. The compound of claim 1, wherein Ar is independently phenyl or pyridinyl, optionally substituted with one or more substituents R^{A}.

5. The compound of claim 1, wherein R^{A} is independently selected from —F, —OMe, and —CO₂Me.

6. The compound of claim 1, wherein L is L¹.

7. The compound of claim 1, wherein L^{1A} is -L^{B1}-O—, wherein -L^{B}- is independently saturated C₁₋₄ alkylene.

8. The compound of claim 1, wherein L^{B} is —CH₂CH₂— or —CH₂CH₂CH₂—.

9. The compound of claim 1 wherein R^{N1} is —H.

10. A compound selected from the following compounds and pharmaceutical acceptable salts thereof:

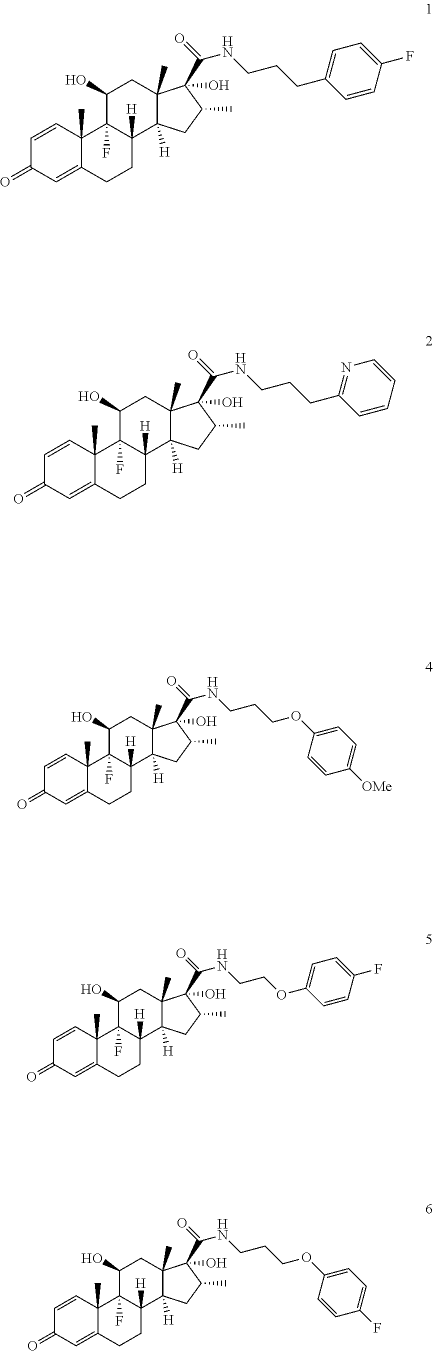

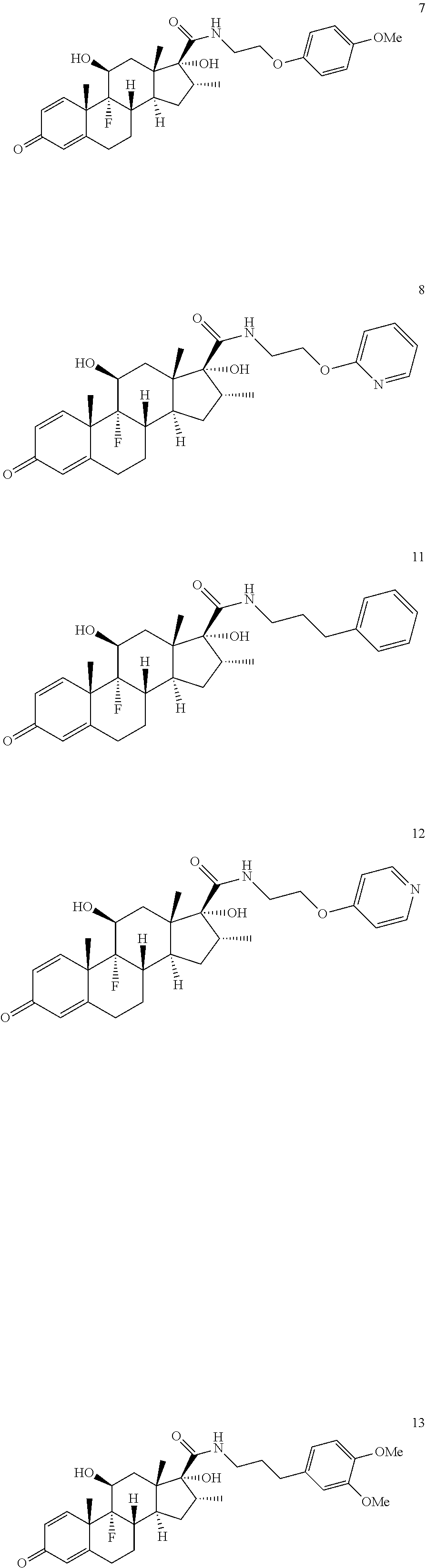

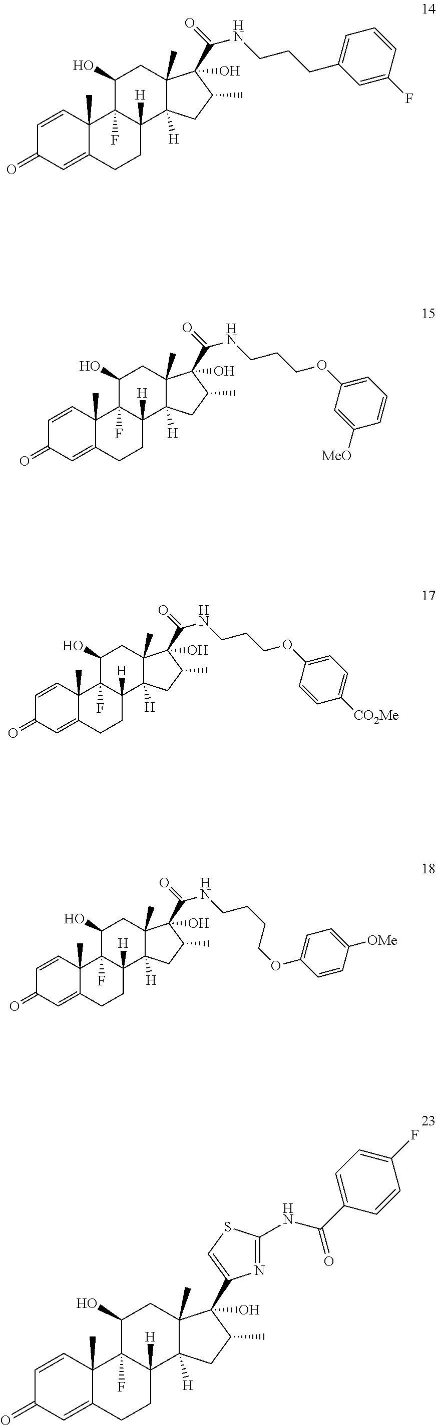

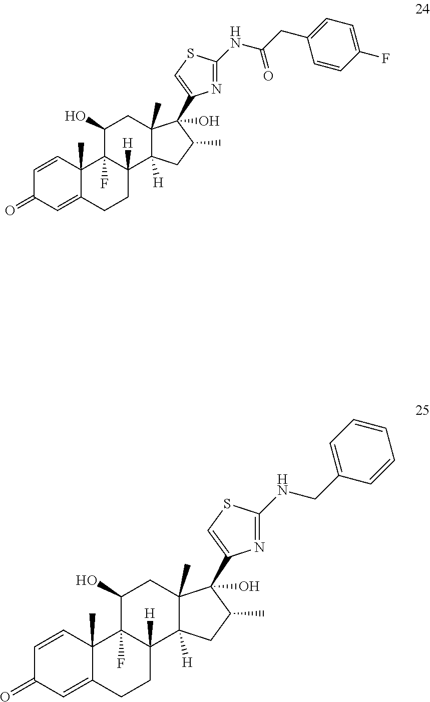

11.-12. (canceled)

13. A method of treating a medical condition selected from an inflammatory condition, a rheumatoid disease, a malignancy, a vascular disease or a lung disease, comprising administering to a subject in need thereof a therapeutically effective amount of the compound of claim 1.

14. The method of claim 13 wherein treating the medical condition comprises one or more of (i) inducing apoptosis in target cells in the subject (ii) causing immunosuppression in the subject and (iii) preventing or treating transplant rejection in the subject.

15. The method of claim 13 wherein the medical condition is selected from a haematological or other malignancy, rheumatoid arthritls, ankylosing, spondylitis, psoriatic arthropathy, systemic lupus, erythematosis, scleroderma, temporal arteritis., polyarteritis nodosa, inflammatory bowel disease, Crohns disease, ulcerative colitis, asthma, chronic obstructive airway disease, and polymyalgia rheumatica.

16. The method of claim 15 which comprises inducing apoptosis in target cells in the subject, the method further comprising administering the therapeutically effective amount o.f the compound of claim 1 to said target cells or to a vicinity in which the target cells are located.

17. A pharmaceutical composition comprising the compound of claim 1; and a pharmaceutically acceptable carrier or diluent.

18. A kit comprising (a) the compound of claim 1, preferably provided as a pharmaceutical composition and in a suitable container and/or with suitable packaging; and (b) instructions for use, for example, written instructions on how to administer the compound.
